SEQUENCE LISTING (17 sequences)

SEQ ID NO: 1 - protein, 497 aa, Homo sapiens
  source 1..497: note = GBA1 (wildtype)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - DNA, 13625 bp, Homo sapiens
  source 1..13625: mol_type = genomic DNA; note = GBA1 (wildtype)
acagaagtacaatgctgattatgatctgtcagctcagcaaggggcagacaccctggccttcatgtctctcctggaggagaagttgatcccggtgctggtgagtgtgcccagacctcccagcatccatggccagccggggaggggacgggaacacacagacccacacagagactcaggagagcatggaggtcagaagcccaccttgaatcagacaggtgcactggctcagacctgcctgtttcttcctgcccacccaatccaggtacatactttttggatagacaccaagaactacgtagaagtgacccggaagtggtatgcagaggctatgccctttcccctcaacttcttcctgcctggccgcatgcagcggcagtacatggaacggctacagctgctgactggggagcacaggcctgaggacgaggaagagctggagaaggaggtagctctgagaccgggggctattgtatgagatgagccccaaggatgctggccaggaatgggagtgcttaggtgcggaggtggcactgttcccgcagctgcaagcctacctgtgtcgcccctacagctgtaccgagaggctcgggagtgtctgaccctgctctctcagcgcctgggctctcaaaagttcttctttggagatgcgtgagtctgactccaagagggtaatgggtggcttggaagaagatacaggttcagatggagcagctggagctggggctggggctggggctggctcaggctctggataggaggtccctgagacagatactggccctggtgacagtggggctgtgcgtggggccagagccttctcagaggtacaaaagggtagggtgggagggcagccaggcacaggaagggcctgaagagctgtggggcactgagtgtgccctttatgcagccctgggatagagccctattcagggccaggctggcgccacctggggatctctccccataccaggtctagaactgtgtgtcctgtccttccctggtggccgcctgctgcccagagcccacctcccaaggctgactcttcctccagctccatctttaccccttctaccccagtggttctcctccatcccacccttctctctctgctccagccctgcctccttggacgccttcgtcttcagctacttggccctgctgctgcaggcaaagctgcccagtgggaagctgcaggtccacctgcgtgggctgcacaacctctgtgcctattgtacccacattctcagtctctacttcccctgggatggaggtaaggggcagatgggaggggcagccctggggagagtgggcagggatccaagaactagttctcctaacacaccttccttccttgaccctcagctgaggtaccaccgcaacgccagacaccagcaggcccagagactgaggaggagccataccggcgccggaaccagatcctatctgtgctggcaggactggcagccatggtgggctacgccttgctcagcggcattgtctccatccagcgggcaacgcctgctcgggccccaggcacccggaccctgggcatggctgaggaggatgaagaggaatgatttgtcctcacgctcccaagactggtttttctactctcatgcattccagaggcccccgtgcctcctcgttgttggtacagccggacacggggtgctgccacccagaataaagccactcacactgactgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggtgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagagacggtcactcatgcagaggcgggactcagagcccttcctcaagtctcattggtcaagttgaacgaacaagtgtcgctggcgagccggagagagagagagagcgagagcgagagagcgagcgagagagagcgagagagcgagagagcgagcgagagagagagagagagagagaggagccggcgcgagaactacgcatgcgtgtcggcgttttcccgccagcacactgttggtggatgggggcgattgaattcccacagtgagtccagcccaccgaagctcagaggattcctaaccttcctctttagagagcctcaggttagggaacgtccagtgcgcagaagctgccccgtgggaatcccattgtccatcgcctcctaatgtttcgtcctggccgtgccctatcccttcctgaggctgggttgttatgatgctgaattattcaagaagtcttgcagcctgacgccatctctgggcagtgctcctcccacctccctgtcttccttggagggcaccacgttgcccctacaagcacagggtcctgaagctgtttacagggccccaccctgccactttagtatcttaacgaatgtttgttgaggacctactgagtgttagacccactgcaatgagcaagtttctgatcttgggaccttaacattctatgggacgacacagatatataaacaaaaataataattttagatagtgatacatgctgaaaccatgggtatgtggccaggcgcggtgagtcacacctgtaatcccagcactttgggagaccgaggcaggcggattacttgaggtcaggagtttgagacccgcctggccaacatggtgaaaccctatctcttctaaaaatacaaaatttagctgggcgggcatggggacgggcgcctgtaatcccagctactcgggaggctgaggcaggagaattgcttgaacccaggaggtggaaggttgcagtgagcccaaatcacaccactgcactccagcctgggtaacaaagcaagaccctgtctcaaaaaaagacaaaatgaaacaaacaaacaaaaaaaaacatggctatgtgatagaatgactgcgatggggattgggtgctcactgaaatggtgagaatggagctaaaacctgatattaggtgatagcaccagatataggggccaagggtttcatgcttggaaaacagcaggtgcaaacgtccttgtgctgtgaggaaatttggctggaacagaaggtgggccagattgtagctgaccatagtctctgggagtttggagatgattctaaatacaatgggaagccattaaattgaggcagagccttgatgtgacgtgctttatcatcattttgctgagtggagaatggattgctgggaaaagtgaaagactgattcagaggttgtggagagaggatggctggagctaggtagctggggaggtggtgggatgtggatgaattcaggataggattggttgtgttggatatgggaaaagaagtcaccatggttcctgagcttttggaatgtgcaaatgggtaatggaggtgccattgagatgggaacagtggaaaaataaccggtgctggggtgggaatcaagagttcagttgtggccatgttgaatctgaaatgcccattagacatccaagcagacatgctgagttggatggcgctcat

SEQ ID NO: 3 - protein, 497 aa, Homo sapiens
  source 1..497: note = GBA1 (N370S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITSLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 4 - protein, 497 aa, Homo sapiens
  source 1..497: note = GBA1 (L444P mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDPDAVALMHPDGSAVVVVLNRSSKDVPLTIKDPAVGFLETISPGYSIHTYLWRRQ

SEQ ID NO: 5 - protein, 497 aa, Homo sapiens
  source 1..497: note = GBA1 (E326K mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGKTHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - DNA, 1611 bp, synthetic construct
  source 1..1611: mol_type = other DNA; note = GBA1 codon-optimized nucleotide sequence
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtag

SEQ ID NO: 7 - DNA, 1662 bp, synthetic construct
  source 1..1662: mol_type = other DNA; note = GBA1 codon-optimized nucleotide sequence with reporter sequence
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagggcggcggcggcagccacaggtggtgctgccccggctgctgcaagaccttctag

SEQ ID NO: 8 - protein, 497 aa, Homo sapiens
  source 1..497: note = GBA1 (T369M mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIIMNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - protein, 497 aa, Homo sapiens
  source 1..497: note = GBA1 (G377S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVSWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 497 aa, Homo sapiens
  source 1..497: note = GBA1 (D409H mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKHTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - protein, 497 aa, Homo sapiens
  source 1..497: note = GBA1 (R120W mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIWVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 12 - protein, 497 aa, Homo sapiens
  source 1..497: note = GBA1 (V394L mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWLRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 13 - protein, 497 aa, Homo sapiens
  source 1..497: note = GBA1 (R496H mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRHQ

SEQ ID NO: 14 - protein, 497 aa, Homo sapiens
  source 1..497: note = GBA1 (K178T mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASTWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 15 - protein, 497 aa, Homo sapiens
  source 1..497: note = GBA1 (R329C mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHCLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 16 - protein, 497 aa, Homo sapiens
  source 1..497: note = GBA1 (L444R mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDRDAVALMHPDGSAVVVVLNRSSKDVPLTIKDPAVGFLETISPGYSIHTYLWRRQ

SEQ ID NO: 17 - protein, 497 aa, Homo sapiens
  source 1..497: note = GBA1 (N188S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTSGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

(19) United States
(12) Patent Application Publication
(10) Pub. No.: US 2025/0082686 A1
(43) Pub. Date: Mar. 13, 2025

(54) AAV-BASED MODULATION OF GBA1 AND RELATED COMPOSITIONS AND USES THEREOF

(71) Applicant: Aspen Neuroscience, Inc., San Diego, CA (US)

(72) Inventors: Andres BRATT-LEAL, San Diego, CA (US); Ai ZHANG, San Diego, CA (US)

(73) Assignee: Aspen Neuroscience, Inc., San Diego, CA (US)

(21) Appl. No.: 18/580,582

(22) PCT Filed: Jul. 20, 2022
(86) PCT No.: PCT/US2022/073973

§ 371 Date: Jan. 18, 2024

Related U.S. Application Data
(60) Provisional application No. 63/272,631, filed on Oct. 27, 2021.
(60) Provisional application No. 63/224,398, filed on Jul. 21, 2021.

(51) Int. Cl. A61K 35/30; A61K 38/47; A61P 25/16; C12N 5/0793; C12N 15/86

(52) U.S. Cl. CPC A61K 35/30; A61K 38/47; A61P 25/16; C12N 5/0619; C12N 15/86; C12Y 302/01045; C12N 2501/15; C12N 2501/155; C12N 2501/16; C12N 2501/42; C12N 2501/727; C12N 2506/1307; C12N 2750/14143

(57) ABSTRACT

The present disclosure provides adeno-associated vector (AAV)-based methods of increasing gene expression of a GBA1 in floor plate midbrain progenitor cells, determined dopamine (DA) neuron progenitor cells, and/or DA neurons, or glial cells, including those differentiated from pluripotent stem cells, and methods of lineage specific differentiation of the same. Also provided are compositions and uses thereof, such as for treating neurodegenerative diseases and conditions, including Parkinson's disease.

### CROSS-REFERENCE TO RELATED APPLICATIONS

This application claims priority to U.S. provisional application 63/224,398, filed Jul. 21, 2021, entitled “AAV-BASED MODULATION OF GBA1 AND RELATED COMPOSITIONS AND USES THEREOF,” and U.S. provisional application 63/272,631, filed Oct. 27, 2021, entitled “AAV-BASED MODULATION OF GBA1 AND RELATED COMPOSITIONS AND USES THEREOF,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 165622001040SeqList.xml, created Jul. 20, 2022, which is 42,603 bytes in size. The information in the electronic format of the Sequence Listing is incorporated by reference in its entirety.

### FIELD

The present disclosure relates to adeno-associated virus (AAV) vector-based methods of increasing gene expression of glucosylceramidase beta (GBA1) gene in floor plate midbrain progenitor cells, determined dopamine (DA) neuron progenitor cells, and/or dopamine (DA) neurons, or glial cells, including those differentiated from pluripotent stem cells, including induced pluripotent stem cells. Also provided are compositions of the cells having increased expression of GBA1 and therapeutic uses thereof, such as for treating neurodegenerative conditions and diseases, including Parkinson's disease.

### BACKGROUND

Reduced activity of certain proteins, including β-Glucocerebrosidase (GCase) (encoded by the glucosylceramidase beta (GBA1) gene), has been associated with an increased risk of developing certain neurodegenerative diseases or disorders, such as Parkinson's Disease (PD). Other diseases or disorders are also associated with reduced GCase activity, such as Gaucher's disease. In some cases, a variant in a gene encoding a protein may contribute to, or cause, reduced activity of the protein. Various methods for differentiating pluripotent stem cells into lineage specific cell populations and the resulting cellular compositions are contemplated to find use in cell replacement therapies for patients with diseases resulting in a loss of function of a defined cell population. However, in some cases, such methods are limited in their ability to produce cells with consistent physiological characteristics, and cells resulting from such methods may be limited in their ability to engraft and innervate other cells in vivo. Moreover, in some cases, such methods involve the use of cells having reduced activity of GCase, such as due to a gene variant, e.g., a SNP, in GBA1 that is associated with an increased risk of developing PD. Improved methods and cellular compositions thereof are needed, including to provide for improved methods for increasing GBA1 expression and/or increasing GCase activity in such differentiated cells.

### SUMMARY

Provided herein are methods of increasing expression of GBA1 in a cell, the methods including: introducing, into a neurally differentiated cell, a recombinant adeno-associated viral (rAAV) vector containing a promoter operably linked to a nucleic acid sequence encoding GBA1, wherein the introducing results in increased expression of GBA1 in the cell.

In some embodiments, prior to the introducing, the cell exhibits reduced activity of GCase. In some embodiments, the cell endogenously contains a variant of GBA1. In some embodiments, the cell is heterozygous for the GBA1 variant. In some embodiments, the cell endogenously contains a variant of GBA1 associated with Parkinson's Disease.

In some embodiments, the cell comprises biallelic variants in GBA1 or is homozygous for the GBA1 variant. In some embodiments, the cell comprises biallelic variants in GBA1. In some embodiments, the cell is homozygous for the GBA1 variant. In some embodiments, the cell endogenously contains one or more variant(s) of GBA1 associated with Gaucher's disease (GD).

Also provided herein are methods of increasing expression of GBA1 in a cell, the method including: introducing, into a neurally differentiated cell, a recombinant adeno-associated viral (rAAV) vector containing a promoter operably linked to a nucleic acid sequence encoding GBA1, wherein the cell contains a variant of GBA1 associated with Parkinson's Disease, and the introducing results in increased expression of GBA1 in the cell. In some embodiments, prior to the introducing, the cell exhibits reduced activity of GCase. In some embodiments, the cell is heterozygous for the GBA1 variant.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence havingat least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portfion thereof. In some embodiments, a functional GCase enzyme or a portion thereof has the enzymatic activity of a wild-type GCase enzyme. In some embodiments, the enzymatic activity of GCase is determined by any of the methods described herein.

In some embodiments, the nucleic acid sequence is a deoxyribonucleic acid (DNA) sequence.

In some embodiments, the rAAV vector is pseudotyped with capsid proteins of an AAV serotype. In some embodiments, the capsid proteins are of the AAV2, AAV3, AAV3B, AAV3H, AAVLK-03, AAV5, AAV6, AAV7m8, AAV8, or AAV9 serotype. In some embodiments, the capsid proteins are of the AAV7m8, AAV9, or AAV-LK03 serotype. In some embodiments, the capsid proteins are of the AAV9 serotype. In some embodiments, the capsid proteins are of the AAV7m8 serotype. In some embodiments, the capsid proteins are of the AAV-LK03 serotype.

In some embodiments, the nucleic acid sequence encoding GBA1 is positioned between inverted terminal repeat (ITRs). In some embodiments, the ITRs are of the same serotype as the capsid proteins.

In some embodiments, the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter. In some embodiments, the promoter is a UBC promoter.

In some embodiments, the neurally differentiated cell exhibits decreased expression of GBA1 prior to being introduced with the rAAV vector, as compared to a reference cell. In some embodiments, the neurally differentiated cell exhibits reduced activity of the β-Glucocerebrosidase (GCase) enzyme encoded by GBA1 prior to being introduced with the rAAV vector, as compared to a reference cell. In some embodiments, the reference cell does not contain a GBA1 variant. In some embodiments, the reference cell does not exhibit decreased GCase activity. In some embodiments, the cell is from a subject who does not exhibit reduced GCase activity. In some embodiments, the reference cell is a cell from a subject without a Lewy body disease (LBD). In some embodiments, the reference cell is a cell from a subject without Parkinson's disease. In some embodiments, the reference cell is a cell from a subject without Gaucher's disease.

In some embodiments, GBA1 is human GBA1. In some embodiments, the nucleic acid sequence comprises the sequence set forth in SEQ ID NO:6 or SEQ ID NO:7. In some embodiments, the nucleic acid sequence comprises the sequence set forth in SEQ ID NO:6. In some embodiments, the nucleic acid sequence comprises the sequence set forth in SEQ ID NO:7. In some embodiments, the nucleic acid sequence encodes an amino acid sequence comprising the amino acid sequence set forth in SEQ ID NO:1.

In some embodiments, the variant of GBA1 is a single nucleotide polymorphism (SNP) that is associated with Parkinson's disease. In some embodiments, the cell is heterozygous for the GBA1 variant.

In some embodiments, the variant of GBA1 contains a single nucleotide polymorphism (SNP) that is associated with Gaucher's disease. In some embodiments, the cell is homozygous for the GBA1 variant. In some embodiments, the cell comprises biallelic variants in GBA1.

In some embodiments, the SNP is rs76763715. In some embodiments, the rs76763715 is a cytosine variant. In some embodiments, the variant of GBA1 comprising a SNP encodes a serine, rather than an asparagine, at amino acid position 370 (N370S). In some embodiments, the wild-type form of GBA1 comprises a thymine instead of the cytosine variant.

In some embodiments, the SNP is rs421016. In some embodiments, the rs421016 is a guanine variant. In some embodiments, the variant of GBA1 comprising the SNP encodes a proline, rather than a leucine, at amino acid position 444 (L444P). In some embodiments, the wild-type form of GBA1 comprises an adenine instead of the guanine variant.

In some embodiments, the SNP is rs2230288. In some embodiments, the rs2230288 is a thymine variant. In some embodiments, the variant of GBA1 comprising the SNP encodes a lysine, rather than a glutamic acid, at position 326 (E326K). In some embodiments, the wild-type form of GBA1 comprises a cytosine instead of the thymine variant.

In some embodiments, the neurally differentiated cell is a dopaminergic neuron progenitor cell or a dopaminergic neuron. In some embodiments, the cell is derived from a pluripotent stem cell (PSC). In some embodiments, the dopaminergic neuron progenitor cell is derived from a pluripotent stem cell (PSC). In some embodiments, the dopaminergic neuron is derived from a pluripotent stem cell (PSC). In some embodiments, the PSC is an induced pluripotent stem cell (iPSC). In some embodiments, the iPSC is artificially derived from a non-pluripotent cell from a subject. In some embodiments, the non-pluripotent cell is a fibroblast. In some embodiments, the fibroblast exhibits reduced GCase activity. In some embodiments, the subject has a Lewy body disease (LBD). In some embodiments, the subject has Parkinson's disease. In some embodiments, the subject has a Parkinson's disease dementia. In some embodiments, the subject has dementia with Lewy bodies (DLB). In some embodiments, the subject has Gaucher's disease.

In some embodiments, a plurality of the neurally differentiated cells were differentiated from pluripotent stem cells (PSCs) by a method including: (a) performing a first incubation including culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; and (b) performing a second incubation including culturing cells of the spheroid in a substrate-coated culture vessel under conditions to neurally differentiate the cells.

In some embodiments, the plurality of the neurally differentiated cells is a plurality of dopaminergic neuron progenitor cells. In some embodiments, the plurality of the neurally differentiated cells is a plurality of dopaminergic neurons. In some embodiments, the PSCs are induced pluripotent stem cells (iPSCs).

Also provided herein are methods of differentiating neural cells, the methods including: (a) performing a first incubation including culturing pluripotent stem cells (PSCs) in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; (b) performing a second incubation including culturing cells of the spheroid in a substrate-coated culture vessel under conditions to neurally differentiate the cells; and (c) introducing into the neurally differentiated cells a recombinant adeno-associated viral (rAAV) vector containing a promoter operably linked to a nucleic acid sequence encoding GBA1, wherein the introducing results in increased expression of GBA1 in the cell.

Also provided here are methods of differentiating neural cells, the methods including: (a) performing a first incubation including culturing a population of pluripotent stem cells in a first culture vessel, wherein beginning at the initiation of the first incubation (day 0) the cells are exposed to (i) an inhibitor of TGF-β/activin-Nodal signaling; (ii) an inhibitor of bone morphogenetic protein (BMP) signaling; and (b) performing a second incubation comprising culturing cells produced by the first incubation in a second culture vessel under conditions to neurally differentiate the cells; and (iii) introducing into the neurally differentiated cells a recombinant adeno-associated viral (rAAV) vector containing a promoter operably linked to a nucleic acid sequence encoding GBA1, wherein the introducing results in increased expression of GBA1 in the cell. In some embodiments, the cells comprise a variant of GBA1 associated with Parkinson's Disease. In some embodiments, the cells are induced pluripotent stem cells.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described herein.

In some embodiments, prior to the introducing, the cells exhibit reduced activity of GCase. In some embodiments, the cells endogenously contain a GBA1 variant. In some embodiments, the cells are heterozygous for the GBA1 variant. In some embodiments, the cells endogenously contain a variant of GBA1 associated with Parkinson's Disease.

In some embodiments, the cells comprise biallelic variants in GBA1. In some embodiments, the cells are homozygous for the GBA1 variant. In some embodiments, the cells endogenously contain one or more variant(s) of GBA1 associated with Gaucher's disease (GD).

In some embodiments, the neurally differentiated cells exhibit decreased expression of GBA1 prior to being introduced with the rAAV vector, as compared to a reference cell. In some embodiments, the neurally differentiated cells exhibit reduced activity of the β-Glucocerebrosidase (GCase) enzyme encoded by GBA1 prior to being introduced with the rAAV vector, as compared to a reference cell. In some embodiments, the reference cell does not exhibit reduced GCase activity. In some embodiments, the reference cell is a cell from a subject without Parkinson's Disease. In some embodiments, the reference cell is a cell from a subject without Gaucher's Disease.

In some embodiments, the cells are exposed to the inhibitor of TGF-β/activin-Nodal signaling up to a day at or before day 7. In some embodiments, the cells are exposed to the inhibitor of TGF-β/activin-Nodal beginning at day 0 and through day 6, inclusive of each day.

In some embodiments, the cells are exposed to the at least one activator of SHH signaling up to a day at or before day 7. In some embodiments, the cells are exposed to the at least one activator of SHH signaling beginning at day 0 and through day 6, inclusive of each day.

In some embodiments, the cells are exposed to the inhibitor of BMP signaling up to a day at or before day 11. In some embodiments, the cells are exposed to the inhibitor of BMP signaling beginning at day 0 and through day 10, inclusive of each day.

In some embodiments, the cells are exposed to the inhibitor of GSK3β signaling up to a day at or before day 13. In some embodiments, the cells are exposed to the inhibitor of GSK3β signaling beginning at day 0 and through day 12, inclusive of each day.

In some embodiments, culturing the cells under conditions to neurally differentiate the cells includes exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

In some embodiments, the cells are exposed to BAGCT and the inhibitor of Notch signaling beginning on day 11. In some embodiments, the cells are exposed to BAGCT and the inhibitor of Notch signaling beginning at day 11 and until harvest of the neurally differentiated cells. In some embodiments, the neurally differentiated cells are harvested on day 18. In some embodiments, the neurally differentiated cells are harvested on day 20. In some embodiments, the cells are exposed to BAGCT and the inhibitor of Notch signaling beginning at day 11 and until harvest of the neurally differentiated cells on day 18. In some embodiments, the cells are exposed to BAGCT and the inhibitor of Notch signaling beginning at day 11 and until harvest of the neurally differentiated cells on day 20, In some embodiments, the cells are exposed to BAGCT and the inhibitor of Notch signaling beginning at day 11 and until harvest of the neurally differentiated cells on day 25.

In some embodiments, the inhibitor of TGF-β/activin-Nodal signaling is SB431542.

In some embodiments, the at least one activator of SHH signaling is SHH or purmorphamine. In some embodiments, the at least one activator of SHH signaling is SHH. In some embodiments, the at least one activator of SHH signaling is purmorphamine. In some embodiments, the at least one activator of SHH signaling is SHH and purmorphamine.

In some embodiments, the inhibitor of BMP signaling is LDN193189.

In some embodiments, the inhibitor of GSK3β signaling is CHIR99021.

In some embodiments, the cells are introduced with the rAAV vector between about day 14 and about day 20. In some embodiments, the cells are introduced with the rAAV vector on about day 1. In some embodiments, the cells are introduced with the rAAV vector on about day 18.

In some embodiments, the cells are harvested between about day 18 and about day 25. In some embodiments, the cells are harvested between about day 18 and about day 20. In some embodiments, the cells are harvested on about day 18. In some embodiments, the cells are harvested on about day 20.

In some embodiments, the neurally differentiated cell was cryopreserved and subsequently thawed prior to the introducing. In some embodiments, the neurally differentiated cell was thawed about 1 day prior to the introducing. In some embodiments, the method further includes cryopreserving the neurally differentiated cell prior to the introducing. In some embodiments, the cryopreserving comprises formulating the neurally differentiated cell with a cryoprotectant.

In some embodiments, the PSC is an induced pluripotent stem cell (iPSC) derived from a non-pluripotent cell from a subject. In some embodiments, the non-pluripotent cell is a fibroblast. In some embodiments, the fibroblast exhibits reduced GCase activity. In some embodiments, the subject has a Lewy body disease (LBD). In some embodiments, the subject has Parkinson's disease dementia. In some embodiments, the subject has Parkinson's disease. In some embodiments, the subject has dementia with Lewy bodies (DLB). In some embodiments, the subject has Gaucher's disease.

Also provided herein is a cell produced by any of the methods provided herein.

Also provided herein is a modified neurally differentiated cell comprising an exogenous deoxyribonucleic acid (DNA) sequence encoding GBA1, wherein the DNA sequence is episomal in the cell. In some embodiments, the DNA is encoded by an episomal vector. In some embodiments, the episomal vector is an AAV vector.

In some embodiments, the neurally differentiated cell expresses EN1 and CORIN. In some embodiments, the neurally differentiated cell is a committed dopaminergic precursor cell. In some embodiments, the cell is formulated with a cryoprotectant.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described herein.

Also provided herein is a therapeutic composition of cells produced by any of the methods provided herein. In some embodiments, the therapeutic composition further comprises a cryoprotectant. In some embodiments, the cells of the composition express EN1 and CORIN and less than 10% of the total cells in the composition express TH. In some embodiments, less than 5% of the total cells in the composition express TH. In some embodiments, the therapeutic composition is for use in treating a subject. In some embodiments, the subject has a disease or disorder associated with reduced GCase activity. In some embodiments, the subject has a Lewy body disease (LBD). In some embodiments, the subject has Parkinson's disease dementia. In some embodiments, the subject has Parkinson's disease. In some embodiments, the subject has dementia with Lewy bodies (DLB). In some embodiments, the subject has Gaucher's disease.

In some embodiments, the therapeutic composition is for use in a method of treating a LBD. In some embodiments, the therapeutic composition is for use in treating a subject with a LBD. In some embodiments, the therapeutic composition is for use in the manufacture of a medicament for treatment of a LBD. In some embodiments, the LBD is Parkinson's disease dementia. In some embodiments, the LBD is DLB.

In some embodiments, the therapeutic composition is for use in treating Parkinson's disease. In some embodiments, the therapeutic composition is for use in treating a subject with Parkinson's disease.

In some embodiments, the therapeutic composition is for use in a method of treating a subject with reduced GCase activity. In some embodiments, the therapeutic composition is for use in treating a subject with reduced GCase activity. In some embodiments, the therapeutic composition is for use in the manufacture of a medicament for treatment of reduced GCase activity.

In some embodiments, the therapeutic composition is for use in a method of treating a subject with a heterozygous variant of GBA1. In some embodiments, the therapeutic composition is for use in treating a subject with a heterozygous variant of GBA1. In some embodiments, the therapeutic composition is for use in the manufacture of a medicament for treatment of a heterozygous variant of GBA.

In some embodiments, the therapeutic composition is for use in a method of treating Gaucher's disease. In some embodiments, the therapeutic composition is for use in treating a subject with Gaucher's disease. In some embodiments, the therapeutic composition is for use in the manufacture of a medicament for treatment of Gaucher's disease.

In some embodiments, the therapeutic composition is for use in a method of treating a subject with biallelic variants of GBA1. In some embodiments, the therapeutic composition is for use in treating a subject with biallelic variants of GBA1. In some embodiments, the therapeutic composition is for use in the manufacture of a medicament for treatment of biallelic variants of GBA1.

In some embodiments, the therapeutic composition is for use in a method of treating a subject with a homozygous variant of GBA1. In some embodiments, the therapeutic composition is for use in treating a subject with a homozygous variant of GBA1. In some embodiments, the therapeutic composition is for use in the manufacture of a medicament for treatment of a homozygous variant of GBA1.

Also provided herein are methods of treatment including administering to a subject any of the therapeutic compositions provided herein.

In some embodiments, the cells of the therapeutic composition are autologous to the subject. In some embodiments, prior to the administering, the subject has reduced GCase activity. In some embodiments, the subject has a heterozygous variant of GBA1. In some embodiments, the subject has Parkinson's disease. In some embodiments, the subject has a homozygous variant of GBA1 or biallelic variants of GBA1. In some embodiments, the subject has a homozygous variant of GBA1. In some embodiments, the subject has biallelic variants of GBA1. In some embodiments, the subject has Gaucher's disease.

In some embodiments, the administering includes delivering the cells of the therapeutic composition by stereotactic injection. In some embodiments, the administering includes delivering the cells of the therapeutic composition through a catheter. In some embodiments, the cells of the therapeutic composition are delivered to the striatum of the subject.

Also provided herein are uses of any of the compositions provided herein for the treatment of a LBD. In some embodiments, the use is for manufacture of a medication for the the treatment of a LBD. In some embodiments, the LBD is Parkinson's disease dementia. In some embodiments, the LBD is DLB.

Also provided herein are uses of any of the therapeutic compositions provided herein. In some embodiments, the use is for the treatment of Parkinson's Disease. In some embodiments, the use is for manufacture of a medication for the the treatment of Parkinson's Disease.

Also provided herein are uses of any of the compositions provided herein for the treatment of reduced GCase activity. In some embodiments, the use is for manufacture of a medication for the the treatment of reduced GCase activity.

Also provided herein are uses of any of the compositions provided herein for the treatment of Gaucher's disease. In some embodiments, the use is for manufacture of a medication for the the treatment of Gaucher's disease.

Also provided herein are recombinant adeno-associated viral (rAAV) nucleic acid vector for increasing expression of GBA1 in a cell, the vector containing a promoter operably linked to a nucleic acid sequence encoding GBA1, wherein the cell exhibits (i) reduced activity of the β-Glucocerebrosidase (GCase) enzyme encoded by GBA1 and/or (ii) reduced expression of GBA1, compared to a reference cell. In some embodiments, the reference cell is from a subject without Parkinson's Disease.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described herein.

In some embodiments, the cell contains a variant of GBA1 associated with Parkinson's Disease. In some embodiments, the variant of GBA1 is a single nucleotide polymorphism (SNP) that is associated with Parkinson's disease.

In some embodiments, the cell comprises biallelic variants in GBA1 or is homozygous for the GBA1 variant. In some embodiments, the cell comprises biallelic variants in GBA1. In some embodiments, the cell is homozygous for the GBA1 variant. In some embodiments, the cell endogenously contains one or more variant(s) of GBA1 associated with Gaucher's disease (GD).

In some embodiments, the SNP is rs76763715. In some embodiments, the rs76763715 is a cytosine variant. In some embodiments, the variant of GBA1 comprising a SNP encodes a serine, rather than an asparagine, at amino acid position 370 (N370S). In some embodiments, the wild-type form of GBA1 comprises a thymine instead of the cytosine variant.

In some embodiments, the SNP is rs421016. In some embodiments, the rs421016 is a guanine variant. In some embodiments, the variant of GBA1 comprising the SNP encodes a proline, rather than a leucine, at amino acid position 444 (L444P). In some embodiments, the wild-type form of GBA1 comprises an adenine instead of the guanine variant.

In some embodiments, the SNP is rs2230288. In some embodiments, the rs2230288 is a thymine variant. In some embodiments, the variant of GBA1 comprising the SNP encodes a lysine, rather than a glutamic acid, at position 326 (E326K). In some embodiments, the wild-type form of GBA1 comprises a cytosine instead of the thymine variant.

In some embodiments, the cell is a dopaminergic (DA) neuron, a microglia, a macrophage, an astrocyte, an oligodendrocyte, or a hematopoietic stem cell (HSC). In some embodiments, the cell is a dopaminergic (DA) neuron, a microglia, an astrocyte, or an oligodendrocyte. In some embodiments, the cell is a DA neuron. In some embodiments, the cell is a microglia. In some embodiments, the cell is a macrophage. In some embodiments, the cell is an astrocyte. In some embodiments, the cell is an oligodendrocyte. In some embodiments, the cell is a HSC.

In some embodiments, the cell is a dopaminergic neuron progenitor cell or a dopaminergic neuron. In some embodiments, the cell is a dopaminergic neuron progenitor cell. In some embodiments, the cell is a dopaminergic neuron. In some embodiments, the cell is derived from a pluripotent stem cell (PSC). In some embodiments, the PSC is an induced pluripotent stem cell (iPSC). In some embodiments, the cell is derived from an iPSC.

In some embodiments, a plurality of the cells were differentiated from pluripotent stem cells (PSCs) by a method including: (a) performing a first incubation including culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; and (b) performing a second incubation including culturing cells of the spheroid in a substrate-coated culture vessel under conditions to neurally differentiate the cells.

In some embodiments, the cell is a dopaminergic neuron progenitor cell or a dopaminergic neuron. In some embodiments, the cell is a dopaminergic neuron progenitor cell. In some embodiments, the cell is a dopaminergic neuron. In some embodiments, the cell is derived from a pluripotent stem cell (PSC). In some embodiments, the PSC is an induced pluripotent stem cell (iPSC). In some embodiments, the cell is derived from an iPSC.

In some embodiments, the cells are exposed to the inhibitor of TGF-β/activin-Nodal signaling and the at least one activator of SHH signaling up to a day at or before day 7.

In some embodiments, wherein the cells are exposed to the inhibitor of BMP signaling up to a day at or before day 11.

In some embodiments, the cells are exposed to the inhibitor of GSK3β signaling up to a day at or before day 13.

In some embodiments, the culturing the cells under conditions to neurally differentiate the cells includes exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

### BRIEF DESCRIPTION OF THE DRAWINGS

FIG. 1 shows an exemplary non-adherent protocol for the differentiation of pluripotent stem cells into determined dopamine (DA) neuron progenitor cells or DA neurons.

FIG. 2 shows an exemplary adherent protocol for the differentiation of pluripotent stem cells into determined dopamine (DA) neuron progenitor cells or DA neurons.

FIG. 3A shows the expression level of the KIAA0319L gene encoding AAV receptor (AAVR) in iPSCs at day 0 and in differentiated cells at days 16-22, 25, 82, and 108.

FIG. 3B shows the expression level of the RPSA gene encoding laminin receptor (LAMR/RPSA) in iPSCs at day 0 and in differentiated cells at days 16-22, 25, 82, and 108.

FIG. 3C shows the expression level of the MET gene encoding hepatocyte growth factor receptor (HGFR) in iPSCs at day 0 and in differentiated cells at days 16-22, 25, 82, and 108.

FIG. 4A shows the delta Cq value of GBA1 gene transcripts in cells transduced with the AAV9 vector on days 25 and 32, compared to non-transduced cells from a healthy donor (NTC), as measured by qPCR.

FIG. 4B shows the delta Cq value of GBA1 gene transcripts in cells transduced with the AAV7m8 vector on day 25, compared to non-transduced cells from a healthy donor (NTC), as measured by qPCR.

FIG. 4C shows the delta Cq value of GBA1 gene transcripts in cells transduced with the AAV-LK03 vector on day 25, compared to non-transduced cells from a healthy donor (NTC), as measured by qPCR.

FIG. 5A shows GCase activity in cells transduced with the AAV9 vector on day 25 (unless otherwise indicated), as compared to non-transduced cells from another donor having idiopathic Parkinson's Disease (ID-PD), non-transduced cells from a donor having a single nucleotide polymorphism in GBA1 (N370S), and non-transduced cells from Donor 1 (NTC).

FIG. 5B shows GCase activity in cells transduced with the AAV7m8 vector on day 25 (unless otherwise indicated), as compared to non-transduced cells from another donor having idiopathic Parkinson's Disease (ID-PD), non-transduced cells from a donor having a single nucleotide polymorphism in GBA1 (N370S), and non-transduced cells from Donor 1 (NTC).

FIG. 5C shows GCase activity in cells transduced with the AAV-LK03 vector on day 25 (unless otherwise indicated), as compared to non-transduced cells from another donor having idiopathic Parkinson's Disease (ID-PD), non-transduced cells from a donor having a single nucleotide polymorphism in GBA1 (N370S), and non-transduced cells from Donor 1 (NTC).

### DETAILED DESCRIPTION

The present disclosure relates to methods of increasing expression and/or activity of β-Glucocerebrosidase (GCase), such as in a subject having reduced GCase activity and/or a variant of the GBA1 gene encoding GCase, e.g., a gene variant associated with Parkinson's Disease (PD) and/or reduced GCase activity. In some embodiments, the subject has reduced GCase activity. In some embodiments, the subject has a variant of GBA1. In some embodiments, the subject is heterozygous for a variant of GBA1. In some cases, subjects having a genetic variation of GBA1, e.g., a single nucleotide polymorphism (SNP), associated with Parkinson's Disease (PD) exhibit decreased activity of GCase. In some cases, subjects having a genetic variation of GBA1, e.g., one or more single nucleotide polymorphism(s) (SNP), associated with Gaucher's disease (GD) exhibit decreased activity of GCase.

In particular, the present disclosure relates to methods of overexpressing the GBA1 gene by AAV-based methods, including in subjects having reduced activity of GCase to increase expression and/or activity of GCase. In particular, the present disclosure also relates to methods of overexpressing the GBA1 gene by adeno-associated virus (AAV)-based methods, including in subjects having a SNP in the GBA1 gene, to increase expression and/or activity of GCase. The provided methods include use of AAV-based vectors to increase expression of GBA1 and/or GCase activity in cells from a subject with PD, and use of such cells or descendants of such cells in replacement cell therapy for treating PD. In particular embodiments, the cell is a pluripotent stem cell that has undergone lineage specific differentiation, and, in some embodiments, the present disclosure further includes methods of lineage specific differentiation of such pluripotent stem cells.

The overexpressing cells made using the methods provided herein are further contemplated for various uses including, but not limited to, use as a therapeutic to reverse disease of, damage to, or a lack of, a certain cell type, such as dopaminergic (DA) neurons, microglia, astrocytes, or oligodendrocytes, in a patient. In some embodiments, the patient has an LBD, such as Parkinson's disease, Parkinson's disease dementia, or dementia with Lewy bodies. The overexpressing cells made using the methods provided herein are contemplated for various uses including, but not limited to, use as a therapeutic to provide, a certain cell type, such hematopoietic stem cells (HSCs), to a patient. In some embodiments, overexpressing cells such as HSCs or microglia are contemplated for use in treating Gaucher's disease.

Specifically described are methods of overexpressing GBA1 in cells differentiated from pluripotent stem (PS) cells, such as in subjects with decreased activity of GCase and/or a gene variant, e.g., a SNP, in GBA1 that is associated with PD, and methods for differentiating the PS cells into one or more neural cell types. In some embodiments, the subject has decreased GCase activity. In some embodiments, the subject has a variant in GBA1. In some embodiments, the variant is associated with Parkinson's disease. In some embodiments, the variant is associated with Gaucher's disease. In some embodiments, the GBA1 gene is the wild-type form thereof. In some embodiments, the GBA1 gene is a functional GBA1 or a portion thereof.

Parkinson's disease (PD) is a progressive neurodegenerative disorder that primarily affects dopaminergic neurons of the substantia nigra. It is currently the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopamine (DA) neurons resulting in ultimately disabling motor dysfunction.

Mutations in certain genes can increase the risk of developing neurodegenerative diseases, such as PD or Parkinsonism. For instance, certain mutations in the GBA1 gene have been associated with the development of PD and Parkinsonism. Hundreds of mutations have been found throughout the GBA1 gene, including common and rare variants. See Sidransky et al., New England J. Med. (2009) 361 (17): 1651-61. Mutations in GBA1 are hypothesized to lead to degradation of the expressed protein product, glucocerebrosidase (GBA), an enzyme in the lysosome, as well as to disruptions in its lysosomal targeting and performance therein. See Do et al., Mol. Neurodegeneration (2019) 14:36. It has been estimated that at least 7-10% of PD patients have a GBA1 mutation, that GBA1 mutations increase risk for developing PD by 20- to 30-fold, and that 30% of carriers of a GBA1 mutation will develop PD by 80 years of age. See Migdalska-Richards and Schapira, J. Neurochem. (2016); 139 (Suppl 1): 77-90. Further, biallelic or homozygous mutations in GBA1 result in the autosomal recessive lysosomal storage disorder, Gaucher's disease (GD), which can have neurodegenerative features. See Stoker et al., “Pathological Mechanisms and Clinical Aspects of GBA1 Mutation-Associated Parkinson's Disease,” Parkinson's Disease: Pathogenesis and Clinical Aspects, Codon Publications (2018) Chapter 3.

GBA1 mutations that are associated with the development of PD and Parkinsonism include mutations in the GBA1 gene that result in an N370S amino acid change due to the presence of a serine, rather than an asparagine, at amino acid position 370 in the expressed Glucocerebrosidase (GCase) enzyme (e.g., with reference to SEQ ID NO:1). Other mutations in the GBA1 gene that are associated with the development of PD and Parkinsonism include mutations that result in an L444P amino acid change due to the presence of a proline, rather than a leucine, at position 444 in the expressed GCase enzyme, and mutations that result in an E326K amino acid change due to the presence of a lysine, rather than a glutamic acid, at position 326 in the expressed GCase enzyme (e.g., with reference to SEQ ID NO: 1). Additional GBA1 mutations that have been identified as associated with the development of PD and Parkinsonism include any of those as described in Han et al., Int J Neurosci (2016) 126 (5): 415-21 and Sidranksy et al., NEJM (2009) 361:1651-61, such as T369M, G377S, D409H, R496H, R120W, V394L, K178T, R329C, L444R, and N188S.

In some cases, it is contemplated that a subject having a Lewy body disease (LBD) other than PD, such as Parkinson's disease dementia or dementia with Lewy bodies (DLB) may benefit from cells overexpressing GBA1. Accumulation of the protein α-synuclein into insoluble intracellular deposits termed Lewy bodies (LBs) is the characteristic neuropathological feature of LBDs. The influence of lipidosis-causing genetic mutations such as in GBA1 is thought to be two-fold, with both reduced clearance of α-synuclein due to autophagic impairments leading to a state of increased abundance of α-synuclein within cells, and the accumulation of lipids known to promote α-synuclein aggregation. Erskine et al., Acta Neuropathologica (2021) 121:511-26; and Stojkovska et al., Cell Tissue Res (2018) 373:51-60.

It is also contemplated that a subject may have reduced GCase activity without having a known or identified mutation in GBA1.

The provided embodiments address problems related to the use of iPSCs derived from a subject, such as a subject having PD, that exhibited decreased activity of GCase and/or contain a variant in GBA1 that increases the risk of developing PD. The provided embodiments also contemplate that the iPSCs may be derived from any subject exhibiting decreased activity of GCase, such as in the iPSCs. In some embodiments, the subject has reduced GCase activity. In some embodiments, the subject has PD. In some embodiments, the subject has GD.

For instance, a strategy for the treatment of PD includes the differentiation of iPSCs derived from a patient with PD into certain cells, such as dopaminergic (DA) neurons, for autologous transplantation into the patient. However, if the patient's cells exhibit low activity of GCase and/or avariant in GBA1 associated with the development of PD, which may have contributed to the patient's development of PD and need for such cell transplantation, then the transplanted cells, e.g., DA neurons, would contribute to an increased risk of recurrence of PD by containing GCase with lower activity and/or the GBA1 gene variant. Thus, overexpressing the wildtype form of a GBA1 having reduced expression and/or a variant associated with PD in cells differentiated from iPSCs derived from a patient would allow for the benefits of autologous transplantation (e.g., avoiding ethical concerns, and avoiding risks of immune rejection) while reducing the risk of disease recurrence by providing the wildtype gene product capable of carrying out its normal functions.

Moreover, the human GBA1 gene has a pseudogene known as glucosylceramidase beta pseudogene 1 (GBAP1) that is approximately 96% homologous to GBA1. Horowitz et al., Genomics (1989), Vol. 4 (1): 87-96. Specifically, the GBA1 gene, located on 1q21-22, includes 11 exons and is 16 kb upstream from GBAP1. The 85-kb region surrounding GBA is particularly gene-rich, encompassing seven genes and two pseudogenes. Recombination within and around the GBA locus occurs relatively frequently, complicating genotype analyses. Sidransky et al., New England J. Med. (2009) 361 (17): 1651-61. Further, strategies for correcting gene variants in the GBA1 gene through gene editing run the risk of adversely affecting the GBAP1 pseudogene by also targeting its gene sequence due to the homology between GBA1 and GBAP1. Thus, alternative strategies are needed to compensate for GBA1 gene variants and/or low gene expression that do not adversely affect the GBAP1 pseudogene. The provided embodiments include such strategies.

Further, the provided strategies are advantageous in that they do not require that a GBA1 variant be known or identified in a subject, such as is required by other methods (e.g., CRISPR-based methods) that directly target a known variant. Rather, the provided methods can be used to increase expression of GBA1 in cells, without the need to determine whether the cells contain one or more GBA1 variants.

Thus, the provided methods may be useful for increasing GCase activity in any cells having or suspected of having reduced GCase activity. Such cells may be from a subject having or suspected of having an LBD. In some embodiments, the LDB is PD. In some embodiments, the LBD is Parkinson's disease dementia. In some embodiments, the LBD is DLB. Such cells may be from a subject having or suspected of having Parkinson's disease or Gaucher's disease. Such cells may be from a subject having or suspected of having PD. Such cells may be from a subject having or suspected of having Gaucher's disease.

The present disclosure also relates to methods of lineage specific differentiation of pluripotent stem cells (PSCs), such as embryonic stem (ES) cells or induced pluripotent stem cells (iPSCs), as well as overexpression of GBA1 in such cells, such as to increase GCase activity. In some embodiments, GBA1 is the wildtype form or a functional form or portion thereof. Specifically described are methods of directing lineage specific differentiation of PSCs or iPSCs into floor plate midbrain progenitor cells, determined dopamine (DA) neuron progenitor cells (DDPCs), dopamine (DA) neurons, or glial cells, such as microglia, astrocytes, oligodendrocytes, or ependymocytes. The differentiated cells made using the methods provided herein are further contemplated for various uses including, but not limited to, use as a therapeutic to reverse disease of, or damage to, a lack of dopamine neurons in a patient. For example, the pluripotent stem cells differentiated by any of the methods described herein may be differentiated into one or more type sof cells, such as for cell therapy. In some embodiments, the pluripotent stem cells produced by any of the methods described herein are differentiated into hematopoietic stem cells (HSCs), macrophages, neurons, microglia, astrocytes, and/or oligodendrocytes. In some embodiments, the pluripotent stem cells differentiated by any of the methods described herein are differentiated into neurons, microglia, astrocytes, and/or oligodendrocytes. In some embodiments, the pluripotent stem cells are differentiated into neurons, e.g., DA neurons. In some embodiments, the pluripotent stem cells are differentiated into microglia. In some embodiments, the the pluripotent stem cells are differentiated into macrophages. In some embodiments, the the pluripotent stem cells are differentiated into HSCs.

Provided herein are methods for lineage specific differentiation of pluripotent stem cells (PSCs), such as embryonic stem (ES) cells or induced pluripotent stem cells (iPSCs) into floor plate midbrain progenitor cells, determined dopamine (DA) neuron progenitor cells, and/or dopamine (DA) neurons; or into glial cells, such as microglia, astrocytes, oligodendrocytes, or ependymocytes. In some aspects, PSCs are differentiated into floor plate midbrain progenitor cells. In some aspects, such floor plate midbrain progenitor cells are further differentiated into determined dopamine (DA) neuron progenitor cells. In some aspects, such determined dopamine (DA) neuron progenitor cells are further differentiated into dopamine (DA) neurons. In some aspects, PSCs are differentiated into floor plate midbrain progenitor cells, then into determined dopamine (DA) neuron progenitor cells, and finally, into dopamine (DA) neurons.

The provided embodiments address problems related to characteristics of Parkinson's disease (PD) including the selective degeneration of midbrain dopamine (mDA) neurons in patients' brains. Because PD symptoms are primarily due to the selective loss of DA neurons in the substantia nigra of the ventral midbrain, PD is considered suitable for cell replacement therapeutic strategies.

A challenge in developing a cell based therapy for PD has been the identification of an appropriate cell source for use in neuronal replacement. The search for an appropriate cell source is decades-long, and many potential sources for DA neuron replacement have been proposed. Kriks, Protocols for generating ES cell-derived dopamine neurons in Development and engineering of dopamine neurons (eds. Pasterkamp, R. J., Smidt, & Burbach) Landes Biosciences (2008); Fitzpatrick, et al., Antioxid. Redox. Signal. (2009) 11:2189-2208. Several of these sources progressed to early stage clinical trials including catecholaminergic cells from the adrenal medulla, carotid body transplants, or encapsulated retinal pigment epithelial cells. Madrazo, et al., N. Engl. J. Med. (1987) 316:831-34; Arjona, et al., Neurosurgery (2003) 53:321-28; Spheramine trial Bakay, et al., Front Biosci. (2004) 9:592-602. However, those trials largely failed to show clinical efficacy and resulted in poor long-term survival and low DA release from the grafted cells.

Another approach was the transplantation of fetal midbrain DA neurons, such as was performed in over 300 patients worldwide. Brundin, et al., Prog. Brain Res. (2010) 184:265-94; Lindvall, & Kokaia, J. Clin. Invest (2010) 120:29-40. Therapy using human fetal tissue in these patients demonstrated evidence of DA neuron survival and in vivo DA release up to 10 or 20 years after transplantation in some patients. In many patients, though, fetal tissue transplantation fails to replace DA neuronal function. Further, fetal tissue transplantation is plagued by challenges including low quantity and quality of donor tissue, ethical and practical issues surrounding tissue acquisition, and the poorly defined heterogeneous nature of transplanted cells, which are some of the factors contributing to the variable clinical outcomes. Mendez, et al. Nature Med. (2008); Kordower, et al. N. Engl. J. Med. (1995) 332:1118-24; and Piccini, et al. Nature Neuroscience (1999) 2:1137-40. Hypotheses as to the limited efficacy observed in the human fetal grafting trials include that fetal grafting may not provide a sufficient number of cells at the correct developmental stage and that fetal tissue is quite poorly defined by cell type and variable with regard to the stage and quality of each tissue sample. Bjorklund, et al. Lancet Neurol. (2003) 2:437-45. A further contributing factor may be inflammatory host response to the graft. Id.

Stem cell-derived cells, such as pluripotent stem cells (PSCs), are contemplated as a source of cells for applications in regenerative medicine. Pluripotent stem cells have the ability to undergo self-renewal and give rise to all cells of the tissues of the body. PSCs include two broad categories of cells: embryonic stem (ES) cells and induced pluripotent stem cells (iPSCs). ES cells are derived from the inner cell mass of preimplantation embryos and can be maintained indefinitely and expanded in their pluripotent state in vitro. Romito and Cobellis, Stem Cells Int. (2016) 2016:9451492. iPSCs can be obtained by reprogramming (“dedifferentiating”) adult somatic cells to become more ES cell-like, including having the ability to expand indefinitely and differentiate into all three germ layers. Id.

Pluripotent stem cells such as ES cells have been tested as sources for generating engraftable cells. Early studies in the 1990s using mouse ES cells demonstrated the feasibility of deriving specific lineages from pluripotent cells in vitro, including neurons. Okabe, et al., Mech. Dev. (1996) 59:89-102; Bain, et al., Dev. Biol. (1995) 168v342-357. Midbrain DA neurons were generated using a directed differentiation strategy based on developmental insights from early explants studies. Lee, et al., Nat. Biotechnol. (2000) 18v675-679; Ye, et al., Cell (1998) 93:755-66. However, these efforts did not result in cell populations containing high percentages of midbrain DA neurons or cells capable of restoring neuronal function in vivo. Additionally, the resulting populations contained a mixture of cell types in addition to midbrain DA neurons.

Existing strategies for using human PSCs (hPSCs) for cell therapy have not been entirely satisfactory. DA neurons derived from human PSCs generally have displayed poor in vivo performance, failing to compensate for the endogenous loss of neuronal function. Tabar, et al. Nature Med. (2008) 14:379-81; Lindvall and Kokaia, J. Clin. Invest (2010) 120:29-40.

More recently, preclinical studies in which human ES cells were first differentiated into midbrain floor intermediates, and then further into DA neurons, exhibited in vivo survival and led to motor deficit recovery in animal models. Krik et al., Nature (2011) 480:547-51; Kirkeby et al., Cell Rep. (2012) 1:703-14. Despite these advances, the use of embryonic stem cells is plagued by ethical concerns, as well as the possibility that such cells may form tumors in patients. Finally, ES cell-derived transplants may cause immune reactions in patients in the context of allogeneic stem cell transplant.

The use of induced pluripotent stem cells (iPSCs), rather than ES-derived cells, has the advantages of avoiding ethical concerns. Further, derivation of iPSCs from a patient to be treated (i.e., the patient receives an autologous cell transplant) avoids risks of immune rejection inherent in the use of embryonic stem cells. As previous studies revealed that poor standardization of transplanted cell material contributes to high variability, new methods of producing substantial numbers of standardized cells, such as for autologous stem cell transplant, are needed. Lindvall and Kokaia, J. Clin. Invest (2010) 120:29-40.

Thus, existing strategies have not yet proved to be successful in producing a population of differentiated cells for use in engraftment procedures for restoring neuronal function in vivo. Provided herein are methods of differentiating PSCs into determined dopaminergic neuron progenitor cells (DDPCs) and/or DA neurons cells.

Unlike previously reported methods, the differentiated cells produced by the methods described herein demonstrate physiological consistency. Importantly, this physiological consistency is maintained across cells differentiated from different subjects. This method therefore reduces variability both within and among subjects, and allows for better predictability of cell behavior in vivo. These benefits are associated with a successful therapeutic strategy, especially in the setting of autologous stem cell transplant, where cells are generated separately for each patient. Such reproducibility benefits among different subjects may also enable scaling in manufacturing and production processes.

Collectively, the methods described herein, including those for differentiating cells from PS cells from a subject having reduced activity of GCase and/or a gene variant in GBA1, and overexpressing GBA1, can be used in combination to provide the benefits described above.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a small guide RNA), or micro RNA.

The term “gene variant associated with Parkinson's Disease,” or “gene variant associated with PD,” or the like, refers to a variant of a gene, such as a single nucleotide polymorphism (SNP) or a mutation, where the presence of that variant in subjects, in either heterozygous or homozygous form, has been associated with an increased risk of developing Parkinson's Disease for those subjects, as compared to the risk of developing Parkinson's Disease for the general population. The term “SNP associated with Parkinson's Disease,” or “SNP associated with PD,” or “SNP that is associated with PD,” or the like, refers to a single nucleotide polymorphism (SNP), where the presence of that particular SNP in subjects, in either heterozygous or homozygous form, has been associated with an increased risk of developing Parkinson's Disease for those subjects, as compared to the risk of developing Parkinson's Disease for the general population. The increased risk of developing Parkinson's Disease can be an increased risk of developing Parkinson's Disease over the course of a lifetime or by a certain age, such as by, e.g., 40 years of age, 45 years of age, 50 years of age, 55 years of age, 60 years of age, 65 years of age, 70 years of age, 75 years of age, or 80 years of age. The general population can either be the general population worldwide, or the general population in one or more countries, continents, or regions, such as the United States. The extent of the increased risk is not particularly limited and can be, e.g., a risk that is or is at least 0.5-fold, 1-fold, 2-fold, 3-fold, 4-fold, 5-fold, 10-fold, 15-fold, 20-fold, 25-fold, or 30-fold higher than the risk for the general population.

As used herein, the term “stem cell” refers to a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers.

As used herein, the terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the term “reprogramming” refers to the process of dedifferentiating a non-pluripotent cell into a cell exhibiting pluripotent stem cell characteristics.

As used herein, the term “adherent culture vessel” refers to a culture vessel to which a cell may attach via extracellular matrix molecules and the like, and requires the use of an enzyme (e.g., trypsin, dispase, etc.) for detaching cells from the culture vessel. An “adherent culture vessel” is opposed to a culture vessel to which cell attachment is reduced and does not require the use of an enzyme for removing cells from the culture vessel.

As used herein, the term “non-adherent culture vessel” refers to a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g., a neutrally charged and/or hydrophilic hydrogel) and/or a surfactant (e.g., pluronic acid). A non-adherent culture vessel may contain rounded or concave wells, and/or microwells (e.g., Aggrewells™). In some embodiments, a non-adherent culture vessel is an Aggrewell™ plate. For non-adherent culture vessels, use of an enzyme to remove cells from the culture vessel may not be required.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient or diluent, respectively.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, a “subject” is a mammal, such as a human or other animal, and typically is human.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

A “vector,” as used herein, refers to a recombinant plasmid or virus that comprises a nucleic acid to be delivered into a host cell, either in vitro or in vivo.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs (i.e., the recombinant nucleic acid is positioned between two ITRs).

A “recombinant AAV vector (rAAV vector)” refers to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

An “inverted terminal repeat” or “ITR” sequence refers to relatively short sequences found at the termini of viral genomes which are in opposite orientation.

An “AAV inverted terminal repeat (ITR)” sequence is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

### II. Method for Differentiating Cells

Provided herein are methods of differentiating neural cells, such as by subjecting cells, e.g., iPSCs, to any of the methods described herein. Unless otherwise indicated, the methods of differentiation provided herein involve cells, e.g., pluripotent stem cells, such as iPSCs that are introduced with one or more rAAV vectors comprising a promoter operably linked to a nucleic acid sequence encoding GBA1 (i.e., a GBA1-containing transgenes) as described herein in Section III.

In some embodiments, the methods of differentiating neural cells can be methods that differentiate cells, e.g., iPSCs into any neural cell type using any available or known method for inducing the differentiation of cells, e.g., pluripotent stem cells. In some embodiments, the method induces differentiation of the cells, e.g., pluripotent stem cells, into floor plate midbrain progenitor cells, determined dopaminergic (DA) neuron progenitor cells, and/or dopaminergic (DA) neurons. In some embodiments, differentiated cells are introduced with a recombinant adeno-associated viral (rAAV) vector comprising a promoter operably linked to a nucleic acid sequence encoding GBA1 (i.e., a GBA1-encoding transgene). In some embodiments, differentiated cells that have been introduced with a recombinant adeno-associated viral (rAAV) vector comprising a promoter operably linked to a nucleic acid sequence encoding GBA1 (i.e., a GBA1-encoding transgene) are further differentiated. Any available and known method for inducing differentiation of the cells, e.g., pluripotent stem cells, into floor plate midbrain progenitor cells, determined dopaminergic (DA) neuron progenitor cells, and/or dopaminergic (DA) neurons can be used, including any of those described, e.g., in Section II.A.

In some embodiments, the method induces differentiation of the cells, e.g., pluripotent stem cells, into glial cells. In some embodiments, the glial cells are selected from the group consisting of microglia, astrocytes, oligodendrocytes, and ependymocytes.

In some embodiments, the method induces differentiation of the cells, e.g., pluripotent stem cells, into microglia or microglial-like cells. Any available and known method for inducing differentiation of the cells, e.g., pluripotent stem cells, into microglia or microglial-like cells can be used. Exemplary methods of inducing differentiation of pluripotent stem cells into microglia or microglial-like cells can be found in, e.g., Abud et al., Neuron (2017), Vol. 94:278-293; Douvaras et al., Stem Cell Reports (2017), Vol. 8:1516-1524; Muffat et al., Nature Medicine (2016), Vol. 22 (11): 1358-1367; and Pandya et al., Nature Neuroscience (2017), Vol. 20 (5): 753-759, the contents of which are hereby incorporated by reference in their entirety. Exemplary methods of inducing differentiation of pluripotent stem cells into microglia can also include, in some embodiments, the use of commercially available kits, and provided methods for use of such kits, including, e.g., STEMdiff™ Microglia Differentiation Kit, Catalog #100-0019 (STEMCELL Technologies, Cambridge, MA).

In some embodiments, the method induces differentiation of the cells, e.g., pluripotent stem cells, into macrophages. Any available and known method for inducing differentiation of the cells, e.g., pluripotent stem cells, into macrophages can be used. Exemplary methods of differentiation of pluripotent stem cells into macrophages can be found in, e.g., Lyadova et al., Front. Cell Dev. Biol., (2021) 9:640703; Mukherjee et al., Methods Mol Biol (2018) 1784:13-28; and Vaughan-Jackson et al., Stem Cell Reports (2021) 16 (7): 1735-48. Exemplary methods of inducing differentiation of pluripotent stem cells into macrophages can also include, in some embodiments, the use of commercially available kits and products, and provided methods for use of such kits and products, including, e.g., ImmunoCult™-SF Macrophage Medium, Catalog #10961 (STEMCELL Technologies, Cambridge, MA); CellXVivo Human M1 Macrophage Differentiation Kit, Cataolog #CDK012 (R&D Systems, Minneapolis, MN); and CellXVivo Human M2 Macrophage Differentiation Kit, Catalog #CDK013 ((R&D Systems, Minneapolis, MN).

In some embodiments, the method induces differentiation of the cells, e.g., pluripotent stem cells, into hematopoietic stem cells (HSCs). Any available and known method for inducing differentiation of the cells, e.g., pluripotent stem cells, into HSCs can be used. Exemplary methods of differentiation of pluripotent stem cells into HSCs can be found in, e.g., Demirci et al., Stem Cells Transl Med. (2020) 9 (12): 1549-57; Alsayegh et al., Curr Genomics. (2019) 20 (6): 438-52; Tan et al., PNAS (2018) 115 (9): 2180-85; and Suzuki et al., Mol Ther (2013) 21 (7): 1424-31. Exemplary methods of inducing differentiation of pluripotent stem cells into HSCs can also include, in some embodiments, the use of commercially available kits and products, and provided methods for use of such kits and products, including, e.g., STEMdiff™ Hematopoietic Kit, Catalog #05310 (STEMCELL Technologies, Cambridge, MA).

In some embodiments, the method induces differentiation of the cells, e.g., pluripotent stem cells, into astrocytes. Any available and known method for inducing differentiation of the cells, e.g., pluripotent stem cells, into astrocytes can be used. Exemplary methods of inducing differentiation of the cells, e.g., pluripotent stem cells, into astrocytes can be found in, e.g., TCW et al., Stem Cell Reports (2017), Vol. 9:600-614, including the methods described in the references cited therein, e.g., in Table 1, the contents of which are hereby incorporated by reference in their entirety. Exemplary methods of inducing differentiation of pluripotent stem cells into astrocytes can include, in some embodiments, the use of commercially available kits, and provided methods for use of such kits, including, e.g., Astrocyte Medium, Catalog #1801 (ScienCell Research Laboratories, Carlsbad, CA); Astrocyte Medium, Catalog #A1261301 (ThermoFisher Scientific Inc, Waltham, MA); and AGM Astrocyte Growth Medium BulletKit, Catalog #CC-3186 (Lonza, Basel, Switzerland), the contents of which are hereby incorporated by reference in their entirety.

In some embodiments, the method induces differentiation of the cells, e.g., pluripotent stem cells, into oligodendrocytes. Any available and known method for inducing differentiation of the cells, e.g., pluripotent stem cells, into oligodendrocytes can be used. Exemplary methods of inducing differentiation of pluripotent stem cells into oligodendrocytes can be found in, e.g., Ehrlich et al., PNAS (2017), Vol. 114 (11): E2243-E2252; Douvaras et al., Stem Cell Reports (2014), Vol. 3 (2): 250-259; Stacpoole et al., Stem Cell Reports (2013), Vol. 1 (5): 437-450; Wang et al., Cell Stem Cell (2013), Vol. 12 (2): 252-264; and Piao et al., Cell Stem Cell (2015), Vol. 16 (2): 198-210, the contents of which are hereby incorporated by reference in their entirety.

### A. Neural Differentiation

Provided herein are methods comprising the differentiation of pluripotent stem cells, e.g., iPSCs, into neural cells. The methods of differentiating neural cells are not limited and can be any available or known method for inducing the differentiation of pluripotent stem cells into floor plate midbrain progenitor cells, determined dopaminergic (DA) neuron progenitor cells, and/or dopaminergic (DA) neurons. Exemplary methods of differentiating neural cells can be found, e.g., in WO2013104752, WO2010096496, WO2013067362, WO2014176606, WO2016196661, WO2015143342, US20160348070, the contents of which are hereby incorporated by reference in their entirety.

Provided herein are methods of differentiating neural cells, involving (1) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; and (b) performing a second incubation including culturing cells of the spheroid in a substrate-coated culture vessel under conditions to neurally differentiate the cells.

The provided methods of differentiating neural cells, such as by subjecting iPSCs to cell culture methods that induce their differentiation into floor plate midbrain progenitor cells, determined dopaminergic (DA) neuron progenitor cells, and/or, dopaminergic (DA) neurons.

As described herein, iPSCs were generated from fibroblasts of human patients with Parkinson's disease. In a first incubation, the iPSCs were then differentiated to midbrain floor plate precursors and grown as spheroids in a non-adherent culture by exposure to small molecules, such as LDN, SB, PUR, SHH, CHIR, and combinations thereof, beginning on day 0. The resulting spheroids were then transferred to an adherent culture as part of a second incubation, optionally following dissociation of the spheroid, before being exposed to additional small molecules (e.g., LDN, CHIR, BDNF, GDNF, ascorbic acid, dbcAMP, TGFβ3, DAPT, and combinations thereof) to induce further differentiation into engraftable determined DA neuron progenitor cells or DA neurons. The provided methods may include any of those described in PCT/US2021/013324, which is incorporated herein by reference in its entirety.

Also provided herein are methods of differentiating neural cells, comprising differentiating pluripotent stem cells, such as any of the cells produced by the methods as described, e.g., in Section II, using any of the methods disclosed in any one of WO2013104752, WO2010096496, WO2013067362, WO2014176606, WO2016196661, WO2015143342, and US20160348070.

Also provided are methods of differentiating neural cells, involving: exposing pluripotent stem cells to (a) an inhibitor of bone morphogenetic protein (BMP) signaling; (b) an inhibitor of TGF-β/activin-Nodal signaling; and (c) at least one activator of Sonic Hedgehog (SHH) signaling. In some embodiments, the method further comprising exposing the pluripotent stem cells to at least one inhibitor of GSK3B signaling. In some embodiments, the exposing to an inhibitor of BMP signaling and the inhibitor of TGF-β/activin-Nodal signaling occurs while the pluripotent stem cells are attached to a substrate. In some embodiments, the inhibitor of BMP signaling is any inhibitor of BMP signaling described herein, the inhibitor of TGF-β/activin-Nodal signaling is any inhibitor of TGF-β/activin-Nodal signaling described herein, and the at least one activator of SHH signaling is any activator of SHH signaling described herein. In some embodiments, during the exposing to the inhibitor of BMP signaling, the inhibitor of TGF-β/activin-Nodal signaling, and the at least one activator of SHH signaling, the pluripotent stem cells are attached to a substrate. In some embodiments, during the exposing to the at least one inhibitor of GSK3β signaling, the pluripotent stem cells are attached to a substrate. In some embodiments, during the exposing to the inhibitor of BMP signaling, the inhibitor of TGF-β/activin-Nodal signaling, and the at least one activator of SHH signaling, the pluripotent stem cells are in a non-adherent culture vessel under conditions to produce a cellular spheroid. In some embodiments, during the exposing to the at least one inhibitor of GSK3B signaling, the pluripotent stem cells are in a non-adherent culture vessel under conditions to produce a cellular spheroid.

### 1. Non-Adherent Culture

The provided methods include culturing PSCs (e.g., iPSCs) by incubation with certain molecules (e.g., small molecules) to induce their differentiation into floor plate midbrain progenitor cells, determined dopamine (DA) neuron progenitor cells, and/or, dopamine (DA) neurons. In particular, in some embodiments, the provided embodiments include a first incubation of PSCs under non-adherent conditions to produce spheroids, in the presence of certain molecules (e.g., small molecules), which can, in some aspects, improve the consistency of producing physiologically relevant cells for implantation. In some embodiments, the methods include performing a first incubation involving culturing pluripotent stem cells in a non-adherent culture vessel under conditions to produce a cell spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling.

In some embodiments, a non-adherent culture vessel is a culture vessel with a low or ultra-low attachment surface, such as to inhibit or reduce cell attachment. In some embodiments, culturing cells in a non-adherent culture vessel does not prevent all cells of the culture from attaching the surface of the culture vessel.

In some embodiments, a non-adherent culture vessel is a culture vessel with an ultra-low attachment surface. In some aspects, an ultra-low attachment surface may inhibit cell attachment for a period of time. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a 6-well or 24-well plate. In some embodiments, the wells of the multi-well plate further include micro-wells. In some any of the provided embodiments, a non-adherent culture vessel, such as a multi-well plate, has round or concave wells and/or microwells. In any of the provided embodiments, a non-adherent culture vessel, such as a multi-well plate, does not have corners or seams.

In some embodiments, a non-adherent culture vessel allows for three-dimensional formation of cell aggregates. In some embodiments, iPSCs are cultured in a non-adherent culture vessel, such as a multi-well plate, to produce cell aggregates (e.g., spheroids). In some embodiments, iPSCs are cultured in a non-adherent culture vessel, such as a multi-well plate, to produce cell aggregates (e.g., spheroids) on about day 7 of the method. In some embodiments, the cell aggregate (e.g., spheroid) expresses at least one of PAX6 and OTX2 on or by about day 7 of the method.

In some embodiments, the first incubation includes culturing pluripotent stem cells in a non-adherent culture vessel under conditions to produce a cellular spheroid.

In some embodiments, the number of PSCs plated on day 0 of the method is between about between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the number of PSCs plated on day 0 of the method is between about 1×10⁵ pluripotent stem cells per well and about 20×10⁶ pluripotent stem cells per well, between about 1×10⁵ pluripotent stem cells per well and about 15×10⁶ pluripotent stem cells per well, between about 1×10⁵ pluripotent stem cells per well and about 10×10⁶ pluripotent stem cells per well, between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 20×10⁶ pluripotent stem cells per well, between about 5×10⁵ pluripotent stem cells per well and about 15×10⁶ pluripotent stem cells per well, between about 5×10⁵ pluripotent stem cells per well and about 10×10⁶ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 6-well plate on day 0 of the method is between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 24-well plate on day 0 of the method is between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, or between about 1×10⁶ pluripotent stem cells per well and about 5×10⁶ pluripotent stem cells per well.

In some days, the number of PSCs plated on day 0 of the method is a number of cells sufficient to produce a cellular spheroid containing between about 1,000 cells and about 5,000 cells, or between about 2,000 cells and about 3,000 cells. In some days, the number of PSCs plated on day 0 of the method is a number of cells sufficient to produce a cellular spheroid containing between about 1,000 cells and about 5,000 cells. In some days, the number of PSCs plated on day 0 of the method is a number of cells sufficient to produce a cellular spheroid containing between about 2,000 cells and about 3,000 cells. In some days, the number of PSCs plated on day 0 of the method is a number of cells sufficient to produce a cellular spheroid containing about 2,000 cells. In some days, the number of PSCs plated on day 0 of the method is a number of cells sufficient to produce a cellular spheroid containing about 3,000 cells. In some embodiments, the spheroids containing the desired number is produced by the method on or by about day 7.

In some embodiments of the method provided herein, the first incubation includes culturing pluripotent stem cells in a non-adherent culture vessel under conditions to produce a cellular spheroid. In some embodiments, the first incubation is from about day 0 through about day 6. In some embodiments, the first incubation comprises culturing pluripotent stem cells in a culture media (“media”). In some embodiments, the first incubation comprises culturing pluripotent stem cells in the media from about day 0 through about day 6. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 6. In some embodiments, the serum replacement is provided in the media at 5% (v/v) on day 0 and day 1, and at 2% (v/v) from day 2 through day 6.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activin-Nodal signaling, at least one activator of Sonic Hedgehog (SHH) signaling, an inhibitor of bone morphogenetic protein (BMP) signaling, an inhibitor of glycogen synthase kinase 3β (GSK3B) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 0.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

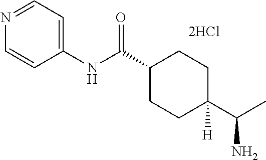 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 0.

In some embodiments the media is supplemented with an inhibitor of TGF-β/activin-Nodal signaling. In some embodiments the media is supplemented with an inhibitor of TGF-β/activin-Nodal signaling up to about day 7 (e.g., day 6 or day 7). In some embodiments the media is supplemented with an inhibitor of TGF-β/activin-Nodal signaling from about day 0 through day 6, each day inclusive.

In some embodiments, cells are exposed to the inhibitor of TGF-β/activin-Nodal signaling at a concentration of between about 1 μM and about 20 μM, between about 5 μM and about 15 μM, or between about 8 μM and about 12 μM. In some embodiments, cells are exposed to the inhibitor of TGF-β/activin-Nodal signaling at a concentration of between about 1 μM and about 20 μM. In some embodiments, cells are exposed to the inhibitor of TGF-β/activin-Nodal signaling at a concentration of between about 5 μM and about 15 μM. In some embodiments, cells are exposed to the inhibitor of TGF-β/activin-Nodal signaling at a concentration of between about 8 μM and about 12 μM. In some embodiments, cells are exposed to the inhibitor of TGF-β/activin-Nodal signaling at a concentration of about 10 μM.

In some embodiments, the inhibitor of TGF-β/activin-Nodal signaling is a small molecule. In some embodiments, the inhibitor of TGF-β/activin-Nodal signaling is capable of lowering or blocking transforming growth factor beta (TGFβ)/Activin-Nodal signaling. In some embodiments, the inhibitor of TGF-β/activin-Nodal signaling inhibits ALK4, ALK5, ALK7, or combinations thereof. In some embodiments, the inhibitor of TGF-β/activin-Nodal signaling inhibits ALK4, ALK5, and ALK7. In some embodiments, the inhibitor of TGF-β/activin-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activin-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

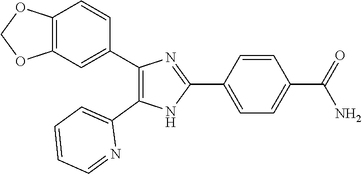 [SMILES: NC(=O)c1ccc(-c2nc(-c3ccc4c(c3)OCO4)c(-c3ccccn3)[nH]2)cc1]

In some embodiments, cells are exposed to SB431542 at a concentration of about 10 μM. In some embodiments, cells are exposed to SB431542 at a concentration of about 10 μM until about day 7. In some embodiments, cells are exposed to SB431542 at a concentration of about 10 μM from about day 0 through about day 6, inclusive of each day.

In some embodiments the media is supplemented with at least one activator of sonic hedghehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). In some embodiments the media is supplemented with the at least one activator of SHH signaling up to about day 7 (e.g., day 6 or day 7). In some embodiments the media is supplemented with the at least one activator of SHH signaling from about day 0 through day 6, each day inclusive.

In some embodiments, the at least one activator of SHH signaling is SHH protein. In some embodiments, the at least one activator of SHH signaling is recombinant SHH protein. In some embodiments, the at least one activator of SHH signaling is recombinant mouse SHH protein. In some embodiments, the at least one activator of SHH signaling is recombinant human SHH protein. In some embodiments, the least one activator of SHH signaling is a recombinant N-Terminal fragment of a full-length murine sonic hedgehog protein capable of binding to the SHH receptor for activating SHH. In some embodiments, the at least one activator of SHH signaling is C25II SHH protein.

In some embodiments, cells are exposed to the at least one activator of SHH signaling at a concentration of between about 10 ng/ml and about 500 ng/mL, between about 20 ng/ml and 400 μg/mL, between about 30 ng/ml and about 300 ng/ml, between about about 40 ng/ml and about 200 ng/mL, or between about 50 ng/mL and about 100 ng/mL, each inclusive. In some embodiments, cells are exposed to the at least one activator of SHH signaling at a concentration of between about 50 ng/ml and about 100 ng/mL, each inclusive. In some embodiments, cells are exposed to the at least one activator of SHH signaling at a concentration of about 100 ng/mL. In some embodiments, the cells are exposed to SHH protein at about 100 ng/mL. In some embodiments, the cells are exposed to recombinant SHH protein at about 100 ng/mL. In some embodiments, the cells are exposed to recombinant mouse SHH protein at about 100 ng/mL. In some embodiments, the cells are exposed to C25II SHH protein at about 100 ng/mL.

In some embodiments, cells are exposed to recombinant SHH protein at a concentration of about 10 ng/mL. In some embodiments, cells are exposed to recombinant SHH protein at a concentration of about 10 ng/ml up to about day 7 (e.g., day 6 or day 7). In some embodiments, cells are exposed to recombinant SHH protein at a concentration of about 10 ng/mL from about day 0 through about day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g., CAS 483367-10-8), having the formula below:

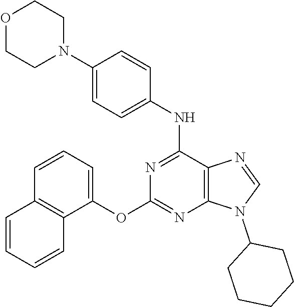 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM up to day 7 (e.g., day 6 or day 7). In some embodiments, cells are exposed to purmorphamine at a concentration of about 10 μM from about day 0 through about day 6, inclusive of each day.

In some embodiments, the at least one activator of SHH signaling is SHH protein and purmorphamine. In some embodiments, cells are exposed to SHH protein and purmorphamine at a concentration up to about day 7 (e.g., day 6 or day 7). In some embodiments, cells are exposed to SHH protein and purpomorphamine from about day 0 through about day 6, inclusive of each day. In some embodiments, cells are exposed to 100 ng/mL SHH protein and 10 μM purmorphamine at a concentration up to about day 7 (e.g., day 6 or day 7). In some embodiments, cells are exposed to 100 ng/ml SHH protein and 10 μM purpomorphamine from about day 0 through about day 6, inclusive of each day.

In some embodiments the media is supplemented with an inhibitor of BMP signaling. In some embodiments the media is supplemented with an inhibitor of BMP signaling up to about day 7 (e.g., day 6 or day 7). In some embodiments the media is supplemented with an inhibitor of BMP signaling from about day 0 through day 6, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

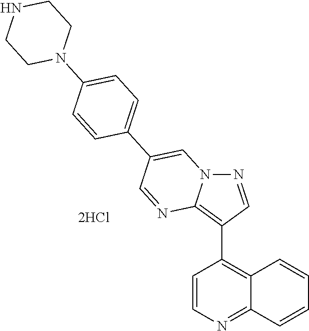 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM up to about day 7 (e.g., day 6 or day 7). In some embodiments, cells are exposed to LDN193189 at a concentration of about 0.1 μM from about day 0 through about day 6, inclusive of each day.

In some embodiments the media is supplemented with an inhibitor of GSK3β signaling. In some embodiments the media is supplemented with an inhibitor of GSK3β signaling up to about day 7 (e.g., day 6 or day 7). In some embodiments the media is supplemented with an inhibitor of GSK3B signaling from about day 0 through day 6, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of BMP signaling at a concentration of between about 1 μM and about 4 μM. In some embodiments, cells are exposed to the inhibitor of BMP signaling at a concentration of between about 2 μM and about 3 μM. In some embodiments, cells are exposed to the inhibitor of GSK3β signaling at a concentration of about 2 μM.

In some embodiments, the inhibitor of GSK3B signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3α. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50-6.7 nM against human GSK3B. In some embodiments, the inhibitor of GSK3B signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

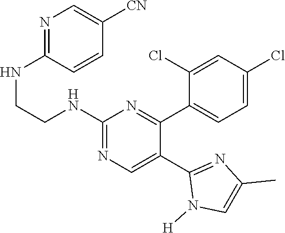 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM up to about day 7 (e.g., day 6 or day 7). In some embodiments, cells are exposed to CHIR99021 at a concentration of about 2.0 μM from about day 0 through about day 6, inclusive of each day.

In some embodiments, from day about 2 to about day 6, at least about 50% of the media is replaced daily. In some embodiments, from about day 2 to about day 6, about 50% of the media is replaced daily, every other day, or every third day. In some embodiments, from about day 2 to about day 6, about 50% of the media is replaced daily. In some embodiments, at least about 75% of the media is replaced on day 1. In some embodiments, about 100% of the media is replaced on day 1. In some embodiments, the replacement media contains small molecules about twice as concentrated as compared to the concentration of the small molecules in the media on day 0.

In some embodiments, the first incubation comprises culturing pluripotent stem cells in a “basal induction media.” In some embodiments, the first incubation comprises culturing pluripotent stem cells in the basal induction media from about day 0 through about day 6. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the small molecules as described above.

### 2. Transfer or Dissociation of Spheroids

In some embodiments, cell aggregates (e.g., spheroids) that are produced following the first incubation of culturing pluripotent stem cells in a non-adherent culture vessel are transferred or dissociated, prior to carrying out a second incubation of the cells on a substrate (adherent culture).

In some embodiments, the first incubation is carried out to produce a cell aggregate (e.g., a spheroid) that expresses at least one of PAX6 and OTX2. In some embodiments, the first incubation produces a cell aggregate (e.g., a spheroid) that expresses PAX6 and OTX2. In some embodiments, the first incubation produces a cell aggregate (e.g., a spheroid) on or by about day 7 of the methods provided herein. In some embodiments, the first incubation produces a cell aggregate (e.g., a spheroid) that expresses at least one of PAX6 and OTX2 on or by about day 7 of the methods provided herein. In some embodiments, the first incubation produces a cell aggregate (e.g., a spheroid) that expresses PAX6 and OTX2 on or by about day 7 of the methods provided herein.

In some embodiments, the cell aggregate (e.g., spheroid) produced by the first incubation is dissociated prior to the second incubation of the cells on a substrate. In some embodiments, the cell aggregate (e.g., spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about day 7. In some embodiments, the cell aggregate (e.g., spheroid) is dissociated by enzymatic dissociation. In some embodiments, the enzyme is selected from among the group consisting of: accutase, dispase, collagenase, and combinations thereof. In some embodiments, the enzyme comprises accutase. In some embodiments, the enzyme is accutase. In some embodiments, the enzyme is dispase. In some embodiments, the enzyme is collagenase.

In some embodiments, the cell aggregate or cell suspension produced therefrom is transferred to a substrate-coated culture vessel for a second incubation. In some embodiments, the cell aggregate (e.g., spheroid) or cell suspension produced therefrom is transferred to a substrate-coated culture vessel following dissociation of the cell aggregate (e.g., spheroid). In some embodiments, the transferring is carried out immediately after the dissociating. In some embodiments, the transferring is carried out on about day 7.

In some embodiments, the cell aggregate (e.g., spheroid) is not dissociated prior to a second incubation. In some embodiments, a cell aggregate (e.g., spheroid) is transferred in its entirety to a substrate-coated culture vessel for a second incubation. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about day 7.

In some embodiments, the transferring is to an adherent culture vessel. In some embodiments, the culture vessel is a plate, a dish, a flask, or a bioreactor. In some embodiments, the culture vessel is substrate-coated. In some embodiments, the substrate is a basement membrane protein. In some embodiments, the substrate is selected from laminin, collagen, entactin, heparin sulfate proteoglycans, and combinations thereof. In some embodiments, the substrate is laminin or a fragment thereof. In some embodiments, the substrate is recombinant. In some embodiments, the substrate is recombinant laminin or a fragment thereof. In some embodiments, the substrate-coated culture vessel is exposed to poly-L-ornithine, optionally prior to being used for culturing cells. In some embodiments, the substrate-coated culture vessel is a 6-well or 24-well plate. In some embodiments, the substrate-coated culture vessel is a 6-well plate. In some embodiments, the substrate-coated culture vessel is a 24-well plate.

### 3. Adherent Culture

In some embodiments, the methods include performing a second incubation of the spheroid cells transferred to the substrate-coated culture vessel. In some embodiments, culturing the cells of the spheroid in the substrate-coated culture vessel under adherent conditions induces their differentiation into floor plate midbrain progenitor cells, determined dopamine (DA) neuron progenitor cells, and/or, dopamine (DA) neurons.

In some embodiments, the second incubation involves culturing cells of the spheroid in a culture vessel coated with a substrate including laminin, collagen, entactin, heparin sulfate proteoglycans, or a combination thereof, wherein beginning on day 7, the cells are exposed to (i) an inhibitor of BMP signaling and (ii) an inhibitor of GSK3B signaling; and beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling. In some embodiments, the method further includes harvesting the differentiated cells.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin, collagen, entactin, heparin sulfate proteoglycans, or a combination thereof. In some embodiments, the substrate is laminin or a fragment thereof. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin or a fragment thereof. In some embodiments, the substrate-coated culture vessel is exposed to poly-L-ornithine. In some embodiments, the substrate-coated culture vessel is exposed to poly-L-ornithine prior to being used for cell culture.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a plate. In some embodiments, the non-adherent culture vessel is a 6-well or 24-well plate. In some embodiments, the non-adherent culture vessel is a dish. In some embodiments, the non-adherent culture vessel is a flask. In some embodiments, the non-adherent culture vessel is a bioreactor.

In some embodiments, the substrate-coated culture vessel allows for a monolayer cell culture. In some embodiments, cells derived from the cell aggregate (e.g., spheroid) produced by the first incubation are cultured in a monolayer culture on the substrate-coated plates. In some embodiments, cells derived from the cell aggregate (e.g., spheroid) produced by the first incubation are cultured to produce a monolayer culture of cells positive for one or more of LMX1A, FOXA2, EN1, CORIN, and combinations thereof. In some embodiments, cells derived from the cell aggregate (e.g., spheroid) produced by the first incubation are cultured to produce a monolayer culture of cells, wherein at least some of the cells are positive for EN1 and CORIN. In some embodiments, cells derived from the cell aggregate (e.g., spheroid) produced by the first incubation are cultured to produce a monolayer culture of cells, wherein at least some of the cells are TH+. In some embodiments, at least some cells are TH+ by or on about day 25. In some embodiments, cells derived from the cell aggregate (e.g., spheroid) produced by the first incubation are cultured to produce a monolayer culture of cells, wherein at least some of the cells are TH+FOXA2+. In some embodiments, at least some cells are TH+FOXA2+ by or on about day 25.

In the methods provided herein, the second incubation involves culturing cells of the spheroid in a substrate-coated culture vessel under conditions to induce neural differentiation of the cells. In some embodiments, the cells of the spheroid are plated on the substrate-coated culture vessel on about day 7.

In some embodiments, the number of cells plated on the substrate-coated culture vessel is between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the second incubation is from about day 7 until harvesting of the cells. In some embodiments, the cells are harvested on about day 16 or later. In some embodiments, the cells are harvested between about day 16 and about day 30. In some embodiments, the cells are harvested between about day 18 and about day 25. In some embodiments, the cells are harvested between about day 19 and about day 24. In some embodiments, the cells are harvested on about day 18. In some embodiments, the cells are harvested on about day 19. In some embodiments, the cells are harvested on about day 20. In some embodiments, the cells are harvested on about day 21. In some embodiments, the cells are harvested on about day 22. In some embodiments, the cells are harvested on about day 23. In some embodiments, the cells are harvested on about day 24. In some embodiments, the cells are harvested on about day 25. In some embodiments, the second incubation is from about day 7 until about day 18. In some embodiments, the second incubation is from about day 7 until about day 19. In some embodiments, the second incubation is from about day 7 until about day 20. In some embodiments, the second incubation is from about day 7 until about day 21. In some embodiments, the second incubation is from about day 7 until about day 22. In some embodiments, the second incubation is from about day 7 until about day 23. In some embodiments, the second incubation is from about day 7 until about day 24. In some embodiments, the second incubation is from about day 7 until about day 25.

In some embodiments, the second incubation involves culturing cells derived from the cell aggregate (e.g., spheroid) in a culture media (“media”).

In some embodiments, the second incubation involves culturing the cells in the media from about day 7 until harvest or collection. In some embodiments, cells are cultured in the media to produce determined dopamine (DA) neuron progenitor cells, or dopamine (DA) neurons.

In some embodiments, the media is also supplemented with a serum replacement containing minimal non-human-derived components (e.g., KnockOut™ serum replacement). In some embodiments, the media is supplemented with the serum replacement from about day 7 through about day 10. In some embodiments, the media is supplemented with about 2% (v/v) of the serum replacement. In some embodiments, the media is supplemented with about 2% (v/v) of the serum replacement from about day 7 through about day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 3B (GSK3B) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16. In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

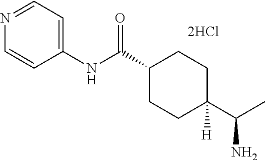 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about 10 μM on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of BMP signaling. In some embodiments the media is supplemented with an inhibitor of BMP signaling from about day 7 up to about day 11 (e.g., day 10 or day 11). In some embodiments the media is supplemented with an inhibitor of BMP signaling from about day 7 through day 10, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is LDN193189 or K02288. In some embodiments, the inhibitor of BMP signaling is capable of inhibiting “Small Mothers Against Decapentaplegic” SMAD signaling. In In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

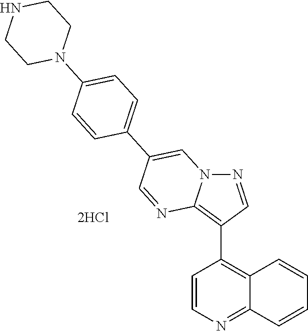 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM from about day 7 up to about day 11 (e.g., day 10 or day 11). In some embodiments, cells are exposed to LDN193189 at a concentration of about 0.1 μM from about day 7 through about day 10, inclusive of each day.

In some embodiments the media is supplemented with an inhibitor of GSK3β signaling. In some embodiments the media is supplemented with an inhibitor of GSK3B signaling from about day 7 up to about day 13 (e.g., day 12 or day 13). In some embodiments the media is supplemented with an inhibitor of GSK3B signaling from about day 7 through day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3B signaling at a concentration of between about 1 μM and about 4 μM. In some embodiments, cells are exposed to the inhibitor of GSK3β signaling at a concentration of between about 2 μM and about 3 μM. In some embodiments, cells are exposed to the inhibitor of GSK3β signaling at a concentration of about 2 μM.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3B signaling is a small molecule. In some embodiments, the inhibitor of GSK3B signaling inhibits a glycogen synthase kinase 3β enzyme. In some embodiments, the inhibitor of GSK3B signaling inhibits GSK3a. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3B. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

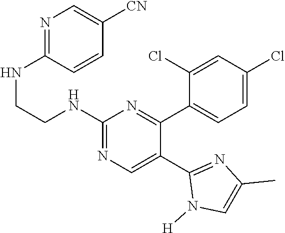 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 7 up to about day 13 (e.g., day 12 or day 13). In some embodiments, cells are exposed to CHIR99021 at a concentration of about 2.0 μM from about day 7 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 20. In some embodiments the media is supplemented with BDNF from about day 11 through day 25.

In some embodiments, cells are exposed to BDNF at a concentration of between about 1 ng/mL and 100 ng/mL, between about 5 ng/mL and about 50 ng/mL, between about 10 ng/ml and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 20. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 20. In some embodiments the media is supplemented with GDNF from about day 11 through day 25.

In some embodiments, cells are exposed to GDNF at a concentration of between about 1 ng/ml and 100 ng/mL, between about 5 ng/mL and about 50 ng/mL, between about 10 ng/ml and about 30 ng/mL. In some embodiments, cells are exposed to GDNF at a concentration of between about 10 ng/ml and about 30 ng/mL. In some embodiments, cells are exposed to GDNF at a concentration of about 20 ng/mL.

In some embodiments, the media is supplemented with about 20 ng/ml GDNF beginning on about day 11. In some embodiments the media is supplemented with 20 ng/ml GDNF from about day 11 until harvest or collection. In some embodiments the media is supplemented with about 20 ng/ml GDNF from about day 11 through day 18. In some embodiments the media is supplemented with about 20 ng/mL GDNF from about day 11 through day 20. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 20. In some embodiments the media is supplemented with ascorbic acid from about day 11 through day 25.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 20. In some embodiments the media is supplemented with about 0.2 mM ascorbic acid from about day 11 through day 25.

In some embodiments the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 20. In some embodiments the media is supplemented with dbcAMP from about day 11 through day 25.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 20. In some embodiments the media is supplemented with about 0.5 mM dbcAMP from about day 11 through day 25.

In some embodiments the media is supplemented with transforming growth factor beta 3 (TGFβ3). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 20. In some embodiments the media is supplemented with TGFβ3 from about day 11 through day 25.

In some embodiments, cells are exposed to TGFβ3 at a concentration of between about 0.1 ng/ml and 10 ng/mL, between about 0.5 ng/mL and about 5 ng/ml, or between about 1.0 ng/ml and about 2.0 ng/mL. In some embodiments, cells are exposed to TGFβ3 at a concentration of between about 1.0 ng/ml and about 2.0 ng/ml, each inclusive. In some embodiments, cells are exposed to TGFβ3 at a concentration of about 1 ng/mL.

In some embodiments, the media is supplemented with about 1 ng/mL TGFβ3 beginning on about day 11. In some embodiments the media is supplemented with 1 ng/ml TGFβ3 from about day 11 until harvest or collection. In some embodiments the media is supplemented with about 1 ng/ml TGFβ3 from about day 11 through day 18. In some embodiments the media is supplemented with about 1 ng/ml TGFβ3 from about day 11 through day 20. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 20. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

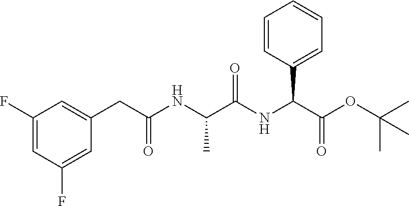 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 μM.

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 20. In some embodiments the media is supplemented with about 10 μM DAPT from about day 11 through day 25.

In some embodiments, the cells are harvested or collected on day 18. In some embodiments, the cells are harvested or collected on day 19. In some embodiments, the cells are harvested or collected on day 20. In some embodiments, the cells are harvested or collected on day 21. In some embodiments, the cells are harvested or collected on day 22. In some embodiments, the cells are harvested or collected on day 23. In some embodiments, the cells are harvested or collected on day 24. In some embodiments, the cells are harvested or collected on day 25.

In some embodiments, beginning on about day 11, the media is supplemented with about 20 ng/mL BDNF, about 20 ng/ml GDNF, about 0.2 mM ascorbic acid, about 0.5 mM dbcAMP, about 1 ng/mL TGFβ3, and about 10 μM DAPT. In some embodiments, from about day 11 until harvest or collection, the media is supplemented with about 20 ng/mL BDNF, about 20 ng/ml GDNF, about 0.2 mM ascorbic acid, about 0.5 mM dbcAMP, about 1 ng/ml TGFβ3, and about 10 μM DAPT. In some embodiments, from about day 11 until day 18, the media is supplemented with about 20 ng/ml BDNF, about 20 ng/ml GDNF, about 0.2 mM ascorbic acid, about 0.5 mM dbcAMP, about 1 ng/ml TGFβ3, and about 10 μM DAPT. In some embodiments, from about day 11 until day 25, the media is supplemented with about 20 ng/mL BDNF, about 20 ng/ml GDNF, about 0.2 mM ascorbic acid, about 0.5 mM dbcAMP, about 1 ng/mL TGFβ3, and about 10 μM DAPT.

In some embodiments, a serum replacement is provided in the media from about day 7 through about day 10. In some embodiments, the serum replacement is provided at 2% (v/v) in the media on day 7 through day 10.

In some embodiments, from day about 7 to about day 16, at least about 50% of the media is replaced daily. In some embodiments, from about day 7 to about day 16, about 50% of the media is replaced daily, every other day, or every third day. In some embodiments, from about day 7 to about day 16, about 50% of the media is replaced daily. In some embodiments, beginning on about day 17, at least about 50% of the media is replaced daily, every other day, or every third day. In some embodiments, beginning on about day 17, at least about 50% of the media is replaced every other day. In some embodiments, beginning on about day 17, about 50% of the media is replaced daily, every other day, or every third day. In some embodiments, beginning on about day 17, about 50% of the media is replaced every other day. In some embodiments, the replacement media contains small molecules about twice as concentrated as compared to the concentration of the small molecules in the media on day 0.

In some embodiments, the second incubation involves culturing cells derived from the cell aggregate (e.g., spheroid) in a “basal induction media.” In some embodiments, the second incubation involves culturing cells derived from the cell aggregate (e.g., spheroid) in a “maturation media.” In some embodiments, the second incubation involves culturing cells derived from the cell aggregate (e.g., spheroid) in the basal induction media, and then in the maturation media.

In some embodiments, the second incubation involves culturing the cells in the basal induction media from about day 7 through about day 10. In some embodiments, the second incubation involves comprises culturing the cells in the maturation media beginning on about day 11. In some embodiments, the second incubation involves culturing the cells in the basal induction media from about day 7 through about day 10, and then in the maturation media beginning on about day 11. In some embodiments, cells are cultured in the maturation media to produce determined dopamine (DA) neuron progenitor cells, or dopamine (DA) neurons.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the molecules described in Section II.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section II.

In some embodiments, the cells are cultured in the basal induction media from about day 7 up to about day 11 (e.g., day 10 or day 11). In some embodiments, the cells are cultured in the basal induction media from about day 7 through day 10, each day inclusive. In some embodiments, the cells are cultured in the maturation media beginning on about day 11. In some embodiments, the cells are cultured in the basal induction media from about day 7 through about day 10, and then the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 19. In some embodiments, cells are harvested on day 20. In some embodiments, cells are harvested on day 21. In some embodiments, cells are harvested on day 22. In some embodiments, cells are harvested on day 23. In some embodiments, cells are harvested on day 24. In some embodiments, cells are harvested on day 25.

### 4. Modulation of GBA1 Expression

In some embodiments, the methods include modulating the expression of GBA1 in neurally differentiated cells prior to their harvest or collection, such as to increase activity of the GCase enzyme. In some embodiments, the activity of Gcase and/or the expression of GBA1 is increased in neurally differentiated cells by any of the methods described in Section III. In some embodiments, the activity of GCase and/or the expression of GBA1 is increased in differentiated cells by introducing into the cells a recombinant adeno-associated viral (rAAV) vector comprising a promoter operably linked to a nucleic acid sequence encoding GBA1 (i.e., a GBA1-containing transgene). In some embodiments, the activity of GCase is increased in differentiated cells by introducing into the cells a recombinant adeno-associated viral (rAAV) vector comprising a promoter operably linked to a nucleic acid sequence encoding GBA1 (i.e., a GBA1-containing transgene). In some embodiments, the expression of GBA1 is increased in differentiated cells by introducing into the cells a recombinant adeno-associated viral (rAAV) vector comprising a promoter operably linked to a nucleic acid sequence encoding GBA1 (i.e., a GBA1-containing transgene).

In some embodiments, the rAAV vector is introduced into the neurally differentiated cells between about day 15 and day 20 of differentiation, such as on day 16. In some embodiments, the rAAV vector is introduced into the neurally differentiated cells on about day 15. In some embodiments, the rAAV vector is introduced into the neurally differentiated cells on about day 16. In some embodiments, the rAAV vector is introduced into the neurally differentiated cells on about day 17. In some embodiments, the rAAV vector is introduced into the neurally differentiated cells on about day 18. In some embodiments, the rAAV vector is introduced into the neurally differentiated cells on about day 19. In some embodiments, the rAAV vector is introduced into the neurally differentiated cells on about day 20.

In some embodiments, cells that have been introduced with a recombinant adeno-associated viral (rAAV) vector comprising a promoter operably linked to a nucleic acid sequence encoding GBA1 (i.e., a GBA1-containing transgene) are subjected to additional differentiation in culture, prior to harvest or collection of the cells. In some embodiments, neurally differentiated cells that have been introduced with the rAAV vector are cultured until between about day 18 and day 25, such as until about day 20. In some embodiments, the rAAV vector is introduced into the neurally differentiated cells on about day 16, and the cells are further differentiated until about day 20, at which time they are harvested or collected. Thus, in some embodiments, the rAAV vector is introduced into the neurally differentiated cells on about day 16, the cells are further differentiated until about day 20, and then the further differentiated cells are harvested or collected on about day 20.

### 5. Harvesting, Collecting, and Formulating Differentiated Cells

In embodiments of the provided methods, neurally differentiated cells produced by the methods provided herein can be harvested or collected, such as for formulation and use of the cells. In some embodiments, the cells are introduced with a recombinant adeno-associated viral (rAAV) vector comprising a promoter operably linked to a nucleic acid sequence encoding GBA1 (i.e., a GBA1-containing transgene) by any of the methods as described in Section III, prior to harvesting or collecting of the differentiated cells. In some embodiments, the provided methods for producing differentiated cells, such as for use as a cell therapy in the treatment of a neurodegenerative disease may include formulation of cells, such as formulation of differentiated cells resulting from the provided methods described herein. In some embodiments, the dose of cells comprising differentiated cells (e.g., determined DA neuron progenitor cells or DA neurons), is provided as a composition or formulation, such as a pharmaceutical composition or formulation. Such compositions can be used in accord with the provided methods, such as in the prevention or treatment of neurodegenerative disorders, including Parkinson's disease.

In some cases, the cells are processed in one or more steps for manufacturing, generating or producing a cell therapy and/or differentiated cells may include formulation of cells, such as formulation of differentiated cells resulting from the methods. In some cases, the cells can be formulated in an amount for dosage administration, such as for a single unit dosage administration or multiple dosage administration.

In certain embodiments, one or more compositions of differentiated cells, including differentiated cells introduced with a recombinant adeno-associated viral (rAAV) vector comprising a promoter operably linked to a nucleic acid sequence encoding GBA1 (i.e., a GBA1-containing transgene), are formulated. In particular embodiments, one or more compositions of differentiated cells, including differentiated cells introduced with the the rAAV vector, are formulated after the one or more compositions have been produced. In some embodiments, the one or more compositions have been previously cryopreserved and stored, and are thawed prior to the administration.

In certain embodiments, the differentiated cells include determined DA neuron progenitor cells. In some embodiments, a formulated composition of differentiated cells is a composition enriched for determined DA neuron progenitor cells. In certain embodiments, the differentiated cells include DA neurons. In some embodiments, a formulated composition of differentiated cells is a composition enriched for DA neurons.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are differentiated for at least 16 days. In some embodiments, the cells are differentiated for between 16 day and 30 days. In some embodiments, the cells are differentiated for between 16 day and 27 days. In some embodiments, the cells are differentiated for between 18 and 25 day. In some embodiments, the cells are differentiated for about 18 days. In some embodiments, the cells are differentiated for about 19 days. In some embodiments, the cells are differentiated for about 20 days. In some embodiments, the cells are differentiated for about 21 days. In some embodiments, the cells are differentiated for about 22 days. In some embodiments, the cells are differentiated for about 23 days. In some embodiments, the cells are differentiated for about 24 days. In some embodiments, the cells are differentiated for about 25 days.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are harvested after at least 16 days of culture. In some embodiments, the cells are harvested between 16 days and 30 days of culture. In some embodiments, the cells are harvested between 16 days and 27 days of culture. In some embodiments, the cells are harvested between 18 days and 25 days of culture. In some embodiments, the cells are harvested after about 18 days of culture. In some embodiments, the cells are harvested after about 19 days of culture. In some embodiments, the cells are harvested after about 20 days of culture. In some embodiments, the cells are harvested after about 21 days of culture. In some embodiments, the cells are harvested after about 22 days of culture. In some embodiments, the cells are harvested after about 23 days of culture. In some embodiments, the cells are harvested after about 24 days of culture. In some embodiments, the cells are harvested after about 25 days of culture.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the differentiated cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the neurodegenerative condition or disease (e.g., Parkinson's disease), such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the differentiated cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of differentiated cells are formulated, cryopreserved, and then stored for an amount of time. In certain embodiments, the formulated, cryopreserved cells are stored until the cells are released for administration. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to differentiated cells. In some embodiments, the volume of formulation buffer is from or from about 1 μL to 5000 μL, such as at least or about at least or about or 5 μL, 10 μL, 20 μL, 50 μL, 100 μL, 200 μL, 300 μL, 400 μL, 500 μL, 1000 μL, 2000 μL, 3000 μL, 4000 μL, or 5000 μL.

A container may generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, such cells produced by the method, or a composition comprising such cells, are administered to a subject for treating a neurodegenerative disease or condition. In some embodiments, the cells produced by the method are introduced with a recombinant adeno-associated viral (rAAV) vector comprising a promoter operably linked to a nucleic acid sequence encoding GBA1 (i.e., a GBA1-containing transgene) by any of the methods as described in Section III. In some embodiments, the cells introduced with the rAAV vector are referred to as “overexpressing cells.”

### B. Exemplary Processes

As described by the methods provided herein, pluripotent stem cells may be differentiated into lineage specific cell populations, including determined DA progenitors cells and DA neurons. These cells may then be used in cell replacement therapy, such as after being introduced with an rAAV vector comprising a promoter operably linked to a nucleic acid sequence encoding GBA1 (i.e., a GBA1-containing transgene).

As described by the methods here, in some embodiments, the pluripotent stem cells are differentiated into floor plate midbrain progenitor cells, and the resulting spheroid cells are further differentiated into determined dopamine (DA) neuron progenitor cells, and/or dopamine (DA) neurons. In some embodiments, the pluripotent stem cells are differentiated into determined DA neuron progenitor cells. In some embodiments, the pluripotent stem cells are differentiated into DA neurons. In some embodiments, pluripotent stem cells are embryonic stem cells. In some embodiments, pluripotent stem cells are induced pluripotent stem cells.

In some embodiments, embryonic stem cells are differentiated into floor plate midbrain progenitor cells, and then into determined dopamine (DA) neuron progenitor cells, and/or dopamine (DA) neurons. In some embodiments, embryonic stem cells are differentiated into determined DA neuron progenitor cells. In some embodiments, embryonic stem cells are differentiated into DA neurons.

In some embodiments, induced pluripotent stem cells are differentiated into floor plate midbrain progenitor cells, and then into determined dopamine (DA) neuron progenitor cells, and/or dopamine (DA) neurons. In some embodiments, induced pluripotent stem cells are differentiated into determined DA neuron progenitor cells. In some embodiments, induced pluripotent stem cells are differentiated into DA neurons.

In some embodiments, the method involves (a) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to induce neural differentiation the cells.

In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the method involves (a) performing a first incubation including culturing pluripotent stem cells in a plate having microwells under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; (iv) an inhibitor of glycogen synthase kinase 3B (GSK3B) signaling; and (v) a serum replacement; (b) dissociating the cells of the spheroid to produce a cell suspension; (c) transferring cells of the cell suspension to a laminin-coated culture vessel; (d) performing a second incubation including culturing cells of the spheroid in the laminin-coated culture vessel under conditions to induce neural differentiation of the cells; and (e) harvesting the neurally differentiated cells. In some embodiments, the second incubation involves culturing cells in the presence of a serum replacement. In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the cells are exposed to the inhibitor of TGF-β/activin-Nodal (e.g., SB431542 or “SB”) from day 0 up to about day 7 (e.g., day 6 or day 7). In some embodiments, the cells are exposed to the inhibitor of TGF-β/activin-Nodal (e.g., SB431542 or “SB”) from day 0 through day 6, inclusive of each day. In some embodiments, the cells are exposed to the at least one activator of SHH signaling (e.g., SHH protein and purmorphamine, collectively “SHH/PUR”) from day 0 up to about day 7 (e.g., day 6 or day 7). In some embodiments, the cells are exposed to the at least one activator of SHH signaling (e.g., SHH protein and purmorphamine, collectively “SHH/PUR”) from day 0 through day 6, inclusive of each day. In some embodiments, the cells are exposed to the inhibitor of BMP signaling (e.g., LDN193189 or “LDN”) from day 0 up to about day 11 (e.g., day 10 or day 11). In some embodiments, the cells are exposed to the inhibitor of BMP signaling (e.g., LDN193189 or “LDN”) from day 0 through day 10, inclusive of each day. In some embodiments, the cells are exposed to the inhibitor of GSK3B signaling (e.g., CHIR99021 or “CHIR”) from day 0 up to about day 13 (e.g., day 12 or day 13). In some embodiments, the cells are exposed to the inhibitor of GSK3B signaling (e.g., CHIR99021 or “CHIR”) from day 0 through day 12.

In some embodiments, the cells are exposed to (i) an inhibitor of TGF-β/activin-Nodal signaling from day 0 up to about day 7 (e.g., day 6 or day 7); (ii) at least one activator of Sonic Hedgehog (SHH) signaling from day 0 up to about day 7 (e.g., day 6 or day 7); (iii) an inhibitor of bone morphogenetic protein (BMP) signaling from day 0 up to about day 11 (e.g., day 10 or day 11); and (iv) an inhibitor of glycogen synthase kinase 3B (GSK3B) signaling from day 0 up to about day 13 (e.g., day 12 or day 13). In some embodiments, the cells are exposed to (i) SB from day 0 up to about day 7 (e.g., day 6 or day 7); (ii) SHH/PUR from day 0 up to about day 7 (e.g., day 6 or day 8); (iii) LDN from day 0 up to about day 11 (e.g., day 10 or day 11); and (iv) CHIR from day 0 up to about day 13 (e.g., day 12 or day 13). In some embodiments, the cells are exposed to (i) an inhibitor of TGF-β/activin-Nodal signaling from day 0 through day 6, each day inclusive; (ii) at least one activator of Sonic Hedgehog (SHH) signaling from day 0 through day 6, each day inclusive; (iii) an inhibitor of bone morphogenetic protein (BMP) signaling from day 0 through day 10, each day inclusive; and (iv) an inhibitor of glycogen synthase kinase 3B (GSK3B) signaling from day 0 through day 12, each day inclusive. In some embodiments, the cells are exposed to (i) SB from day 0 through day 6, each day inclusive; (ii) SHH/PUR from day 0 through day 6, each day inclusive; (iii) LDN from day 0 through day 10, each day inclusive; and (iv) CHIR from day 0 through day 12, each day inclusive.

In some embodiments, the cells are exposed to brain-derived neurotrophic factor (BDNF) beginning on day 11. In some embodiments, the cells are exposed to ascorbic acid. In some embodiments, the cells are exposed to glial cell-derived neurotrophic factor (GDNF) beginning on day 11. In some embodiments, the cells are exposed to dibutyryl cyclic AMP (dbcAMP) beginning on day 11. In some embodiments, the cells are exposed to transforming growth factor beta-3 (TGFβ3) beginning on day 11. In some embodiments, the cells are exposed to the inhibitor of Notch signaling (e.g., DAPT) beginning on day 11. In some embodiments, beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) the inhibitor of Notch signaling (e.g., DAPT) (collectively “BAGCT/DAPT”). In some embodiments, the cells are exposed to BAGCT/DAPT beginning on day 11 until harvest or collection. In some embodiments, the cells are exposed to BAGCT/DAPT from day 11 through day 18. In some embodiments, the cells are exposed to BAGCT/DAPT from day 11 through day 20. In some embodiments, the cells are exposed to BAGCT/DAPT from day 11 through day 25.

In some embodiments, the cells are exposed to a Rho-associated protein kinase (ROCK) inhibitor on day 0. In some embodiments, the cells are exposed to a Rho-associated protein kinase (ROCK) inhibitor on day 7. In some embodiments, the cells are exposed to a Rho-associated protein kinase (ROCK) inhibitor on day 16. In some embodiments, the cells are exposed to a Rho-associated protein kinase (ROCK) inhibitor on day 20. In some embodiments, the cells are exposed to a Rho-associated protein kinase (ROCK) inhibitor on day 0, day 7, day 16, and day 20. In some embodiments, the cells are exposed to a ROCK inhibitor on the day on which the cells are passaged. In some embodiments, the cells are passaged on day 0, 7, 16, 20, or combinations thereof. In some embodiments, the cells are passaged on day 0, 7, 16, and 20.

In some embodiments, the cells are cultured in a basal induction medium comprising DMEM/F-12 and Neurobasal media (e.g., at a 1:1 ratio), supplemented with N2, B27, non-essential amino acids (NEAA), Glutamax, L-glutamine, β-mercaptoethanol, and insulin. In some embodiments, the cells are cultured in the basal induction media from about day 0 through about day 10. In some embodiments, the basal induction media is for differentiating pluripotent stem cells into floor plate midbrain progenitor cells.

In some embodiments, the cells are cultured in a maturation medium comprising Neurobasal media, supplemented with N2, B27, non-essential amino acids (NEAA), and Glutamax. In some embodiments, the cells are cultured in the basal induction media from about day 11 until harvest or collection. In some embodiments, the cells are cultured in the basal induction media from about day 11 through day 18. In some embodiments, the cells are cultured in the basal induction media from about day 11 through day 19. In some embodiments, the cells are cultured in the basal induction media from about day 11 through day 20. In some embodiments, the cells are cultured in the basal induction media from about day 11 through day 21. In some embodiments, the cells are cultured in the basal induction media from about day 11 through day 22. In some embodiments, the cells are cultured in the basal induction media from about day 11 through day 23. In some embodiments, the cells are cultured in the basal induction media from about day 11 through day 24. In some embodiments, the cells are cultured in the basal induction media from about day 11 through day 25. In some embodiments, the maturation media is for differentiating floor plate midbrain progenitor cells into determined dopamine (DA) neuron progenitor cells. In some embodiments, the maturation media is for differentiating floor plate midbrain progenitor cells into dopamine (DA) neurons.

In some embodiments, the media is supplemented with small molecules as described above, including SB, SHH/PUR, LDN, CHIR, BAGCT/DAPT, and ROCKi. In some embodiments, the media is changed every day or every other day. In some embodiments the media is changed every day. In some embodiments the media is changed every other day. In some embodiments, the media is changed every day from about day 0 up to about day 17 (e.g., day 16 or day 18). In some embodiments, the media is changed every other day from about day 18 until harvest or collection. In some embodiments, the media is changed every day from about day 0 up to about day 17 (e.g., day 16 or day 18), and then every other day from about day 18 until harvest or collection. In some embodiments, the cells are harvested or collected on day 20.

In some embodiments, a serum replacement is provided in the media from about day 0 up to about day 10 (e.g., day 9 or day 11). In some embodiments, the serum replacement is provided at 5% (v/v) in the media on day 0 and day 1. In some embodiments, the serum replacement is provided at 2% (v/v) in the media on day 2 through day 10. In some embodiments, the serum replacement is provided at 5% (v/v) in the media on day 0 and day 1 and at 2% (v/v) in the media on day 2 through day 10. In some embodiments, serum replacement is not provided in the media after day 10.

In some embodiments, at least about 50% or at least about 75% of the media is changed. In some embodiments, at least about 50% of the media is changed. In some embodiments, at least about 75% of the media is changed. In some embodiments about 100% of the media is changed.

In some embodiments, about 50% or about 75% of the media is changed. In some embodiments, about 50% of the media is changed. In some embodiments, about 75% of the media is changed. In some embodiments about 100% of the media is changed.

In some embodiments, the media is supplemented with small molecules selected from SB, SHH/PUR, LDN, CHIR, BAGCT/DAPT, ROCKi, or a combination thereof. In some embodiments, when about 50% of the media is changed, the concentration of each small molecule is doubled as compared to its concentration on day 0.

In some embodiments, the cells are introduced with a recombinant adeno-associated viral (rAAV) vector comprising a promoter operably linked to a nucleic acid sequence encoding GBA1 between about day 14 and about day 20. In some embodiments, the cells are introduced with the rAAV vector on about day 16. In some embodiments, the cells are introduced with the rAAV vector on about day 18.

In some embodiments, the cells introduced with the rAAV vector are further differentiated until they are harvested. In some embodiments, the cells are introduced with the rAAV vector on about day 16 and are further differentiated until harvest. In some embodiments, the cells are introduced with the rAAV vector on about day 18 and are further differentiated until harvest.

In some embodiments, cells are harvested between about day 16 and about day 30. In some embodiments, cells are harvested between about day 16 and about day 27. In some embodiments, cells are harvested between about day 18 and about day 25. In some embodiments, cells are harvested between about day 19 and about day 24. In some embodiments, cells are harvested on about day 18. In some embodiments, cells are harvested on about day 19. In some embodiments, cells are harvested on about day 20. In some embodiments, cells are harvested on about day 21. In some embodiments, cells are harvested on about day 22. In some embodiments, cells are harvested on about day 23. In some embodiments, cells are harvested on about day 24. In some embodiments, cells are harvested on about day 25 . . . . In some embodiments, cells harvested on about day 18 are determined DA progenitor cells or DA neurons. In some embodiments, cells harvested on about day 18 are determined DA progenitor cells. In some embodiments, cells harvested on about day 18 are DA neurons. In some embodiments, cells are harvested on about day 20. In some embodiments, cells harvested on about day 20 are determined DA progenitor cells or DA neurons. In some embodiments, cells harvested on about day 20 are determined DA progenitor cells. In some embodiments, cells harvested on about day 20 are DA neurons. In some embodiments, cells are harvested on about day 25. In some embodiments, cells harvested on about day 25 are determined DA progenitor cells or DA neurons. In some embodiments, cells harvested on about day 25 are determined DA progenitor cells. In some embodiments, cells harvested on about day 25 are DA neurons.

In some embodiments, the cells are introduced with the rAAV vector on about day 16 and are further differentiated until harvest on about day 20. In some embodiments, the cells are introduced with the rAAV vector on about day 18 and are further differentiated until harvest on about day 20.

In some embodiments, compositions comprising cells generated by the methods provided herein are used for the treatment of a neurodegenerative disease or condition, such as Parkinson's disease. In some embodiments, a composition of cells generated by any of the methods described herein are administered to a subject who has Parkinson's disease. In some embodiments, a composition of cells generated by any of the methods described herein are administered by stereotactic injection, such as with a catheter. In some embodiments, a composition of cells generated by any of the methods described herein are administered to the striatum of a subject with Parkinson's disease.

Also provided herein is an exemplary method of differentiating neural cells, the method comprising: exposing the pluripotent stem cells to: (a) an inhibitor of bone morphogenetic protein (BMP) signaling; (b) an inhibitor of TGF-β/activin-Nodal signaling; and (c) at least one activator of Sonic Hedgehog (SHH) signaling. In some embodiments, during the exposing, the pluripotent stem cells are attached to a substrate. In some embodiments, during the exposing, the pluripotent stem cells are in a non-adherent culture vessel under conditions to produce a cellular spheroid.

In some embodiments, the method further comprises exposing the pluripotent stem cells to at least one inhibitor of GSK3β signaling. In some embodiments, during the exposing to the at least one inhibitor of GSK3β signaling, the pluripotent stem cells are attached to a substrate. In some embodiments, during the exposing to the at least one inhibitor of GSK3β signaling, the pluripotent stem cells are in a non-adherent culture vessel under conditions to produce a cellular spheroid.

In some embodiments, the inhibitor of TGF-β/activin-Nodal signaling is SB431542.

In some embodiments, the at least one activator of SHH signaling is SHH or purmorphamine. In some embodiments, the inhibitor of BMP signaling is LDN193189. In some embodiments, the at least one inhibitor of GSK3β signaling is CHIR99021.

In some embodiments, the exposing results in a population of differentiated neural cells. In some embodiments, the differentiated neural cells are floor plate midbrain progenitor cells, determined dopamine (DA) neuron progenitor cells, and/or, dopamine (DA) neurons.

The differentiated neural cells produced by any of the methods described herein are sometimes referred to as “differentiated cells.”

### III. Methods of Modulating GBA1 Expression

Provided herein are methods of increasing expression of GBA1 in a cell.

Also provided herein are methods of increasing expression of GBA1 in a cell, the methods including: introducing, into a neurally differentiated cell, a recombinant adeno-associated viral (rAAV) vector comprising a promoter operably linked to a nucleic acid sequence encoding GBA1, wherein the introducing results in increased expression of GBA1 in the cell.

In some embodiments, GBA1 is the wildtype form and/or a functional GBA1 or portion thereof. In some embodiments, GBA1 is the wildtype form thereof. In some embodiments, GBA1 is a functional GBA1 or a portion thereof. In some embodiments, GBA1 is a functional GBA1.

In some embodiments, the cell comprises a variant of GBA1 associated with Parkinson's Disease.

Also provided herein are methods of increasing expression of GBA1 in a cell, the methods including: introducing, into a neurally differentiated cell, a recombinant adeno-associated viral (rAAV) vector comprising a promoter operably linked to a nucleic acid sequence encoding GBA1, wherein the cell comprises a variant of GBA1 associated with Parkinson's Disease, and the introducing results in increased expression of GBA1 in the cell.

Also provided herein are methods of differentiating neural cells, the methods including: (a) performing a first incubation comprising culturing pluripotent stem cells (PSCs) in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; (b) performing a second incubation comprising culturing cells of the spheroid in a substrate-coated culture vessel under conditions to neurally differentiate the cells; and (c) introducing into the neurally differentiated cells a recombinant adeno-associated viral (rAAV) vector comprising a promoter operably linked to a nucleic acid sequence encoding GBA1, wherein the introducing results in increased expression of GBA1 in the cell.

In some embodiments, the PSCs are induced pluripotent stem cells (iPSCs). In some embodiments, the PSCs exhibit reduced activity of GCase. In some embodiments, the PSCs endogenously contain a variant of GBA1. In some embodiments, the PSCs are heterozygous for the GBA1 variant. In some embodiments, the PSCs comprise a variant of GBA1 associated with Parkinson's Disease.

In some embodiments, the PSCs comprise biallelic variants in GBA1 or are homozygous for the GBA1 variant. In some embodiments, the PSCs comprise biallelic variants in GBA1. In some embodiments, the PSCs are homozygous for the GBA1 variant. In some embodiments, the PSCs endogenously contain one or more variant(s) of GBA1 associated with Gaucher's disease (GD).

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described in Section III.D.

In some embodiments, the neurally differentiated cell exhibits decreased expression of GBA1 prior to being introduced with the rAAV vector, as compared to a reference cell. In some embodiments, the neurally differentiated cell exhibits reduced activity of the β-Glucocerebrosidase (GCase) enzyme encoded by GBA1 prior to being introduced with the rAAV vector, as compared to a reference cell.

In some embodiments, the reference cell does not exhibit reduced GCase activity. In some embodiments, the reference cell is a cell from a subject without an LBD. In some embodiments, the LBD is PD. In some embodiments, the LBD is Parkinson's disease dementia. In some embodiments, the LBD is DLB. In some embodiments, the reference cell is a cell from a subject without Parkinson's Disease. In some embodiments, the reference cell is a cell from a subject without Gaucher's Disease.

The provided methods, in some embodiments, result in increased expression of GBA1 in a cell, increased activity of the GCase enzyme encoded by GBA1 in the cell, or both, by introducing an rAAV vector encoding GBA1 into into the cell, thereby resulting in overexpression of GBA1 in the cell. In some embodiments, the method results in increased activity of the GCase enzyme.

In some embodiments, the expression of GBA1 is increased in the cell. In some embodiments, the expression of GBA1 is increased in the cell by about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1,000%. In some embodiments, the expression of GBA1 is increased in the cell by about 10%. In some embodiments, the expression of GBA1 is increased in the cell by about 20%. In some embodiments, the expression of GBA1 is increased in the cell by about 30%. In some embodiments, the expression of GBA1 is increased in the cell by about 40%. In some embodiments, the expression of GBA1 is increased in the cell by about 50%. In some embodiments, the expression of GBA1 is increased in the cell by about 60%. In some embodiments, the expression of GBA1 is increased in the cell by about 70%. In some embodiments, the expression of GBA1 is increased in the cell by about 80%. In some embodiments, the expression of GBA1 is increased in the cell by about 90%. In some embodiments, the expression of GBA1 is increased in the cell by about 100%. In some embodiments, the expression of GBA1 is increased in the cell by about 200%. In some embodiments, the expression of GBA1 is increased in the cell by about 300%. In some embodiments, the expression of GBA1 is increased in the cell by about 400%. In some embodiments, the expression of GBA1 is increased in the cell by about 500%. In some embodiments, the expression of GBA1 is increased in the cell by about 600%. In some embodiments, the expression of GBA1 is increased in the cell by about 700%. In some embodiments, the expression of GBA1 is increased in the cell by about 800%. In some embodiments, the expression of GBA1 is increased in the cell by about 900%. In some embodiments, the expression of GBA1 is increased in the cell by about 1,000%.

In some embodiments, the activity of GCase is increased in the cell. In some embodiments, the activity of GCase is increased in the cell by about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1,000%. In some embodiments, the activity of GCase is increased in the cell by about 10%. In some embodiments, the activity of GCase is increased in the cell by about 20%. In some embodiments, the activity of GCase is increased in the cell by about 30%. In some embodiments, the activity of GCase is increased in the cell by about 40%. In some embodiments, the activity of GCase is increased in the cell by about 50%. In some embodiments, the activity of GCase is increased in the cell by about 60%. In some embodiments, the activity of GCase is increased in the cell by about 70%. In some embodiments, the activity of GCase is increased in the cell by about 80%. In some embodiments, the activity of GCase is increased in the cell by about 90%. In some embodiments, the activity of GCase is increased in the cell by about 100%. In some embodiments, the activity of GCase is increased in the cell by about 200%. In some embodiments, the activity of GCase is increased in the cell by about 300%. In some embodiments, the activity of GCase is increased in the cell by about 400%. In some embodiments, the activity of GCase is increased in the cell by about 500%. In some embodiments, the activity of GCase is increased in the cell by about 600%. In some embodiments, the activity of GCase is increased in the cell by about 700%. In some embodiments, the activity of GCase is increased in the cell by about 800%. In some embodiments, the activity of GCase is increased in the cell by about 900%. In some embodiments, the activity of GCase is increased in the cell by about 1,000%.

### A. Samples, Cells, and Cell Preparations

In embodiments of the provided methods, cells are introduced with a recombinant adeno-associated viral (rAAV) vector comprising a promoter operably linked to a nucleic acid sequence encoding GBA1.

In some embodiments, prior to the introducing, the cells exhibit reduced activity of GCase. In some embodiments, the cells endogenously contains a variant of GBA1. In some embodiments, a variant of GBA1 is associated with PD. In some embodiments, the cells are heterozygous for the GBA1 variant. In some embodiments, the cells contain a variant of GBA1 associated with PD.

In some embodiments, the cells contain biallelic variants in GBA1 or are homozygous for the GBA1 variant. In some embodiments, the cells contain biallelic variants in GBA1. In some embodiments, the cells are homozygous for the GBA1 variant. In some embodiments, the cells endogenously contain one or more variant(s) of GBA1 associated with Gaucher's disease (GD).

In some embodiments, the cell is a pluripotent stem cell that has undergone lineage specific differentiation. In some embodiments, the cell that has undergone lineage specific differentiation is referred to as a “differentiated cell.” In some embodiments, the differentiated cell is a floor plate midbrain progenitor cell, a determined dopaminergic (DA) neuron progenitor cell (DDPCs), a dopaminergic (DA) neuron, or a glial cell, such as a microglia, astrocyte, oligodendrocyte, or ependymocytes. In some embodiments, the differentiated cell is a DDPC.

Differentiated cells can be derived from various sources of pluripotent stem cells, including embryonic stem (ES) cells and induced pluripotent stem cells (iPSCs). In some embodiments, the pluripotent stem cell is an iPSC. In some embodiments, the pluripotent stem cell is an iPSC. In some embodiments, the pluripotent stem cell is an iPSC, artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka, Cell (2006) 126:663-76.

In some embodiments, the pluripotent stem cell that was artificially derived from a non-pluripotent cell of a subject. In some embodiments, the non-pluripotent cell is a fibroblast. In some embodiments, the fibroblast exhibits reduced GCase activity. In some embodiments, the subject is a human. In some embodiments, the subject is a human with an LBD. In some embodiments, the LBD is PD. In some embodiments, the LBD is Parkinson's disease dementia. In some embodiments, the LBD is DLB. In some embodiments, the subject is a human with Parkinson's Disease. In some embodiments, the subject is a human with Gaucher's disease. In some embodiments, the pluripotent stem cell is an iPSC.

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphology associated with pluripotent stem cells.

Methods for generating iPSCs are known. For example, mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the PSCs (e.g., iPSCs) are from a subject having reduced activity of GCase and/or a variant in GBA1, wherein reduced activity of GCase and/or the variant in GBA1 is associated with PD. In some embodiments, the PSCs have reduced activity of GCase (e.g., as compared to cells from a subject not having Parkinson's disease). In some embodiments, the PSCs have a variant in GBA1. In some embodiments, the PSCs have reduced activity of GCase and a variant in GBA1.

In some embodiments, the subject is homozygous for a GBA1 variant or has biallelic GBA1 variants. In some embodiments, the subject is homozygous for a GBA1 variant. In some embodiments, the subject has biallelic GBA1 variants. In some embodiments, the PSCs (e.g., iPSCs) are from a subject having one or more do variant(s) in GBA1 that is associated with GD. The gene variant in GBA1 that is associated with GD is not limited and can be any gene variant, e.g., SNP, in GBA1 that is associated with GD.

In some embodiments, the PSCs (e.g., iPSCs) are from a subject having a gene variant, e.g., SNP, in GBA1 that is associated with PD. The gene variant in GBA1 that is associated with PD is not limited and can be any gene variant, e.g., SNP, in GBA1 that is associated with PD, e.g., is associated with an increased risk of developing PD. The gene variant in GBA1 that is associated with PD can be any gene variant, e.g., SNP, in GBA1 that is associated with reduced activity of GCase. In some embodiments, the gene variant is a mutation in the GBA1 gene that results in an N370S amino acid change due to the presence of a serine, rather than an asparagine, at amino acid position 370 in the expressed GCase enzyme; or is a mutation in the GBA1 gene that results in an L444P amino acid change due to the presence of a proline, rather than a leucine, at position 444 in the expressed GCase enzyme; or is a mutation that results in an E326K amino acid change due to the presence of a lysine, rather than a glutamic acid, at position 326 in the expressed GCase enzyme (e.g., with reference to SEQ ID NO:1). In some embodiments, the gene variant is a SNP in the GBA1 gene selected from the group consisting of rs76763715, rs421016, and rs2230288 (e.g., with reference to SEQ ID NO:2).

In some embodiments, the PSCs (e.g., iPSCs) are autologous to the subject to be treated, i.e., the PSCs are derived from the same subject to whom the differentiated cells that were previously introduced with the rAAV vector are administered.

In some embodiments, non-pluripotent cells (e.g., fibroblasts) having reduced GCase activity are reprogrammed to become iPSCs before integration of one or more GBA1-containing transgene(s) and/or differentiation into neural and/or neuronal cells. In some embodiments, non-pluripotent cells (e.g., fibroblasts) derived from patients having a Lewy body disease (LBD) are reprogrammed to become iPSCs before integration of one or more GBA1-containing transgene(s) and/or differentiation into neural and/or neuronal cells. In some embodiments, the LBD is Parkinson's disease (PD). In some embodiments, the LBD is Parkinson's disease dementia. In some embodiments, the LBD is dementia with Lewy bodies (DLB). In some embodiments, non-pluripotent cells (e.g., fibroblasts) derived from patients having Parkinson's disease (PD) are reprogrammed to become iPSCs before introduction with the rAAV vector and/or before differentiation into neural and/or neuronal cells. In some embodiments, non-pluripotent cells (e.g., fibroblasts) derived from patients having Gaucher's disease (PD) are reprogrammed to become iPSCs before integration of one or more GBA1-containing transgene(s) and/or differentiation into neural and/or neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become iPSCs before lineage specific differentiation, such as by any of the methods described in Section II. In some embodiments, the differentiated cells are determined DA neuron progenitors cells and/or DA neurons. In some embodiments, the differentiated cells are introduced with a recombinant adeno-associated viral (rAAV) vector comprising a promoter operably linked to a nucleic acid sequence encoding GBA1 (i.e., a GBA1-containing transgene). In some embodiments, the differentiated cells introduced with the transgene encoding the wildtype form of GBA1 overexpress the wildtype form of the target gene (“overexpressing cells”). In some embodiments, the resulting overexpressing cells are then administered to the patient from whom they are derived in an autologous stem cell transplant. In some embodiments, the resulting overexpressing cells are allogeneic to the subject to be treated, i.e., the overexpressing cells are derived from a different individual than the subject to whom the overexpressing cells will be administered. In some embodiments, non-pluripotent cells (e.g., fibroblasts) derived from another individual (e.g., an individual not having a neurodegenerative disorder, such as Parkinson's disease) are reprogrammed to become iPSCs and then differentiated into determined DA neuron progenitor cells and/or DA neurons, prior to being introduced with the transgene encoding the wildtype form of a target gene (e.g., GBA1). In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting overexpressing cells are then administered to an individual who is not the same individual from whom the overexpressing cells are derived (e.g., allogeneic cell therapy or allogeneic cell transplantation).

In any of the provided embodiments, the PSCs described herein (e.g., allogeneic cells) may be genetically engineered to be hypoimmunogenic. In some embodiments, methods for reducing the immunogenicity generally include ablating expression of HLA molecules and/or introducing immunomodulatory factors into a safe harbor locus. Newly integrated genes may affect the surrounding endogenous genes and chromatin, potentially altering cell behavior or favoring cellular transformation. Thus, inserting exogenous DNA (e.g., encoding immunomodulatory factors) in a pre-selected locus such as a safe harbor locus, or genomic safe harbor (GSH) is important for safety, efficiency, copy number control, and for reliable gene response control. Safe harbor loci include any of those known in the art, including those described in U.S. Pat. No. 11,072,781, which is incorporated by reference herein in its entirety. In some embodiments, the safe harbor locus may be AAVS1, CCR5, CLYBL, ROSA26, collagen, HTRP, H11, beta-2 microglobulin, GAPDH, PCSK9, TCR or RUNX1. Particular methods for reducing the immunogenicity are known, and include ablating polymorphic HLA-A/-B/-C and HLA class II molecule expression and introducing the immunomodulatory factors PD-L1, HLA-G, and CD47 into the AAVS1 safe harbor locus in differentiated cells. Han et al., PNAS (2019) 116 (21): 10441-46. Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, PSCs (e.g., iPSCs) are cultured in the absence of feeder cells, until they reach 80-90% confluency, at which point they are harvested and further cultured for differentiation (day 0). In one aspect of the method described herein, once iPSCs reach 80-90% confluence, they are washed in phosphate buffered saline (PBS) and subjected to enzymatic dissociation, such as with Accutase™, until the cells are easily dislodged from the surface of a culture vessel. The dissociated iPSCs are then re-suspended in media for downstream differentiation into the desired cell type(s), such as determined DA neuron progenitor cells and/or DA neurons. Section II provides exemplary methods for differentiation of PSCs, e.g., iPSCs.

In some embodiments, the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine,β-mercaptoethanol, and insulin. In some embodiments, the basal induction media is further supplemented with serum replacement, a Rho-associated protein kinase (ROCK) inhibitor, and various small molecules, for differentiation. In some embodiments, the PSCs are resuspended in the same media they will be cultured in for at least a portion of the first incubation. In some embodiments, the differentiated cells are introduced with a recombinant adeno-associated viral (rAAV) vector comprising a promoter operably linked to a nucleic acid sequence encoding GBA1 (i.e., a GBA1-containing transgene). In some embodiments, the differentiated cells introduced with the rAAV vector overexpress the wildtype form of the target gene. In some embodiments, the differentiated cells introduced with the rAAV vector are further differentiated, such as until harvest.

### B. AAV-Based Modulation of GBA1 Expression

The provided methods involve, in some embodiments, introducing a recombinant adeno-associated viral (rAAV) vector comprising a promoter operably linked to a nucleic acid sequence encoding GBA1 (i.e., a GBA1-containing transgene) into a cell, such as a cell differentiated by any of the methods described in Section II.

### 1. AAV Vectors

Provided herein are recombinant adeno-associated viral (rAAV) vectors comprising a promoter operably linked to a nucleic acid sequence encoding GBA1 (i.e., a GBA1-containing transgene). Thus, in some aspects, the disclosure relates to recombinant AAVs (rAAVs) comprising a transgene that encodes GBA1.

Adeno-associated virus (AAV) is a member of the Parvoviridae, a virus family characterized by a single stranded linear DNA genome and a small icosahedral shaped capsid measuring about 20 nm in diameter. AAV was first described as a contamination of tissue culture grown simian virus 15, a simian adenovirus and was found dependent on adenovirus for measurable replication. This lead to its name, adeno-associated virus, and its classification in the genus Dependovirus (reviewed in Hoggan et al., 1970). AAV is a common contaminant of adenovirus samples and has been isolated from human virus samples (AAV2, AAV3, AAV5), from samples of simian virus-15 infected cells (AAV1, AAV4) as well as from stocks of avian (AAAV) (Bossis and Chiorini, 2003), bovine, canine and ovine adenovirus and laboratory adenovirus type 5 stock (AAV6). DNA spanning the entire rep-cap ORFs of AAV7 and AAV8 was amplified by PCR from heart tissue of rhesus monkeys (Gao et al., 2002).

The term “rAAVs” generally refers to viral particles comprising an rAAV vector encapsidated by one or more AAV capsid proteins. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known and unknown AAV serotypes. These AAV components, including inverted terminal repeats (ITRs) may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

An rAAV described by the disclosure may comprise a capsid protein having a serotype selected from AAV1. AAV2, AAV3, AAV4, AAV5, AAV6, AAV7, AAV8, AAV9, AAV10, and AAV-LK03. In certain embodiments, the vector is any human or non-human primate isolate, variant, recombinant, chimeric or AAV capsid, including mutations, substitutions, deletions or additions. In certain embodiments, the adeno-associated viral vector is AAV7 (e.g., AAV7m8), AAV-9, or AAV-LK03. In certain embodiments, the adeno-associated viral vector is AAV-7 (e.g., AAV7m8). In certain embodiments, the adeno-associated viral vector is AAV-9. In certain embodiments, the adeno-associated viral vector is AAV-LK03. In some embodiments, an rAAV comprises a capsid protein from a non-human host, for example a rhesus AAV capsid protein such as AAVrh. 1O, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100) amino acid substitutions (e.g., mutations) relative to the wild-type AAV capsid protein from which it is derived.

The term “rAAV vector” refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs), i.e., the heterologous polynucleotide is positioned between two AAV ITRs. The rAAV vectors described herein can have an inverted terminal repeat sequence (ITR) at each end. For use in the methods described herein, the recombinant hybrid recombinant AAV vector vector genomes will typically have all or a portion of at least one of the ITRs or a functional equivalent, which is generally required for the hybrid recombinant AAV vectors to replicate and be packaged into hybrid recombinant AAV vector particles. A functional equivalent of an ITR is typically an inverted repeat which can form a hairpin structure. The term rAAV vector encompasses both rAAV vector particles and rAAV vector plasmids.

In certain embodiments, the recombinant adeno-associated viral vector (rAAV) is an AAV vector capable of transducing a cell (e.g., a neurally differentiated cell). In some embodiments, the rAAV vector is free of both wildtype and helper virus. In certain embodiments, the rAAV vector comprises a promoter operably linked to a nucleic acid sequence encoding GBA1. In some embodiments, GBA1 is human GBA1. The promoter can be any desired promoter, selected by known considerations, such as the level of expression of a nucleic acid operably linked to the promoter and the cell type in which the vector is to be used. That is, the promoter can be tissue/cell-specific. Promoters can be prokaryotic, eukaryotic, fungal, nuclear, mitochondrial, viral or plant promoters. Promoters can be exogenous or endogenous to the cell type being transduced by the vector. Promoters can include, for example, bacterial promoters, known strong promoters such as SV40 or the inducible metallothionein promoter, or an AAV promoter. Additionally, chimeric regulatory promoters for targeted gene expression can be utilized. Examples of these regulatory systems, which are known in the art, include the tetracycline based regulatory system which utilizes the tet transactivator protein ((TA), a chimeric protein containing the VP 16 activation domain fused to the tet repressor of Escherichia coli, the IPTG based regulatory system, the CID based regulatory system, and the Ecdysone based regulatory system (No, D., et al., Proc Natl Acad Sci USA. 93 (8): 3346-3351 (1996)). Other promoters include promoters derived from actin genes, immunoglobulin genes, cytomegalovirus (CMV), ubiquitin C (UBC) promoter, adenovirus, bovine papilloma virus, adenoviral promoters, such as the adenoviral major late promoter, an inducible heat shock promoter, respiratory syncytial virus, Rous sarcomas virus (RSV), etc. A constitutive promoter may be used, such as a chicken beta actin promoter, a chicken beta actin promoter with a cytomegalovirus enhancer, or modifications thereof, such as modification to shorten the length of the promoter. In some embodiments, the promoter is or comprises a UBC promoter. In some embodiments, the promoter is a UBC promoter. In some embodiments, the rAAV vector provided herein comprises a UBC promoter.

A suitable recombinant adeno-associated virus (AAV) is generated by culturing a host cell which contains a nucleic acid sequence encoding an adeno-associated virus (AAV) serotype capsid protein, or fragment thereof; a functional rep gene; a transgene composed of, at a minimum, AAV inverted terminal repeats (ITRs) and a GBA1 nucleic acid sequence; and sufficient helper functions to permit packaging of the transgene into the AAV capsid protein. The components required to be cultured in the host cell to package an AAV transgene in an AAV capsid may be provided to the host cell in trans. Alternatively, any one or more of the required components (e.g., transgene, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter or a constitutive promoter. The promoter of the host cell can also be the desired promoter for the vector used in the therapeutic compositions, and may depend upon the selected cell which is ultimately to be treated. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The transgene, rep sequences, cap sequences, and helper functions required for producing the rAAV may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al. Molecular Cloning: A Laboratory Manual, Cold Spring Harbor Press, Cold Spring Harbor, N.Y. Similarly, methods of generating rAAV virions are known. See, e.g., K. Fisher et al, 1993 J. Virol., 70:520 to 532 and U.S. Pat. No. 5,478,745. An rAAV can be produced using any suitable method (e.g., using recombinant rep and cap genes).

In some embodiments, the rAAV vector comprises a capsid protein having an AAV9 serotype and a UBC promoter operably linked to a nucleic acid sequence encoding human GBA1. In some embodiments, the nucleic acid sequence encoding human GBA1 is positioned between ITRs.

In some aspects, rep and cap genes are provided in trans together with the adenoviral helper proteins required for AAV genome replication and packaging. Thus, in some embodiments, the methods further comprise introducing into a cell (i) a plasmid expressing rep and cap genes; and/or (ii) a plasmid encoding for adenoviral helper genes. In some embodiments, the methods involve: introducing into a cell (i) a recombinant adeno-associated viral (rAAV) vector comprising a promoter operably linked to a nucleic acid sequence encoding GBA1 (i.e., a GBA1-containing transgene); and (ii) a plasmid expressing rep and cap genes. In some embodiments, the methods involve: introducing into a cell (i) a recombinant adeno-associated viral (rAAV) vector comprising a promoter operably linked to a nucleic acid sequence encoding GBA1 (i.e., a GBA1-containing transgene); and (ii) a plasmid encoding for adenoviral helper genes. In some embodiments, the methods involve introducing into a cell: (i) a recombinant adeno-associated viral (rAAV) vector comprising a promoter operably linked to a nucleic acid sequence encoding GBA1 (i.e., a GBA1-containing transgene); (ii) a plasmid expressing rep and cap genes; and (iii) a plasmid encoding for adenoviral helper genes.

### 2. GBA1-Containing Transgene

Provided herein are recombinant adeno-associated viral (rAAV) vectors comprising a promoter operably linked to a nucleic acid sequence encoding GBA1 (i.e., a GBA1-containing transgene).

In some embodiments, GBA1 is the wild-type form of GBA1. In some embodiments, the nucleic acid sequence encoding GBA1 encodes the wild-type form of human GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO:1 (i.e., GCase). In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described in Section III.D.

In some embodiments, the nucleic acid sequence encoding GBA1 is codon optimized. In some embodiments, the nucleic acid sequence encoding GBA1 is modified by optimization of the codons for expression in humans. Codon optimization generally involves balancing the percentages of codons selected with the abundance, e.g., published abundance, of human transfer RNAs, for example, so that none is overloaded or limiting. In some cases, such balancing is necessary or useful because most amino acids are encoded by more than one codon, and codon usage generally varies from organism to organism. Differences in codon usage between transfected or transduced genes or nucleic acids and host cells can have effects on protein expression from the nucleic acid molecule. Table 1 below sets forth an exemplary human codon usage frequency table. In some embodiments, to generate codon-optimized nucleic acid sequences, codons are chosen to select for those codons that are in balance with human usage frequency. The redundancy of the codons for amino acids is such that different codons code for one amino acid, such as depicted in Table 1. In selecting a codon for replacement, it is desired that the resulting mutation is a silent mutation such that the codon change does not affect the amino acid sequence. Generally, the last nucleotide of the codon (e.g., at the third position) can remain unchanged without affecting the amino acid sequence.

TABLE 1
Human Codon Usage Frequency

| Human | amino | freq./ | | Human | amino | freq./ | |
|---|---|---|---|---|---|---|---|
| codon | acid | 1000 | number | codon | acid | 1000 | number |
| TTT | F | 17.6 | 714298 | TCT | S | 15.2 | 618711 |
| TTC | F | 20.3 | 824692 | TCC | S | 17.7 | 718892 |
| TTA | L | 7.7 | 311881 | TCA | S | 12.2 | 496448 |
| TTG | L | 12.9 | 525688 | TCG | S | 4.4 | 179419 |
| CTT | L | 13.2 | 536515 | CCT | P | 17.5 | 713233 |
| CTC | L | 19.6 | 796638 | CCC | P | 19.8 | 804620 |
| CTA | L | 7.2 | 290751 | CCA | P | 16.9 | 688038 |
| CTG | L | 39.6 | 1611801 | CCG | P | 6.9 | 281570 |
| ATT | I | 16 | 650473 | ACT | T | 13.1 | 533609 |
| ATC | I | 20.8 | 846466 | ACC | T | 18.9 | 768147 |
| ATA | I | 7.5 | 304565 | ACA | T | 15.1 | 614523 |
| ATG | M | 22 | 896005 | ACG | T | 6.1 | 246105 |
| GTT | V | 11 | 448607 | GCT | A | 18.4 | 750096 |
| GTC | V | 14.5 | 588138 | GCC | A | 27.7 | 1127679 |
| GTA | V | 7.1 | 287712 | GCA | A | 15.8 | 643471 |
| GTG | V | 28.1 | 1143534 | GCG | A | 7.4 | 299495 |
| TAT | Y | 12.2 | 495699 | TGT | C | 10.6 | 430311 |
| TAC | Y | 15.3 | 622407 | TGC | C | 12.6 | 513028 |
| TAA | * | 1 | 40285 | TGA | * | 1.6 | 63237 |
| TAG | * | 0.8 | 32109 | TGG | W | 13.2 | 535595 |
| CAT | H | 10.9 | 441711 | CGT | R | 4.5 | 184609 |
| CAC | H | 15.1 | 613713 | CGC | R | 10.4 | 423516 |
| CAA | Q | 12.3 | 501911 | CGA | R | 6.2 | 250760 |
| CAG | Q | 34.2 | 1391973 | CGG | R | 11.4 | 464485 |
| AAT | N | 17 | 689701 | AGT | S | 12.1 | 493429 |
| AAC | N | 19.1 | 776603 | AGC | S | 19.5 | 791383 |
| AAA | K | 24.4 | 993621 | AGA | R | 12.2 | 494682 |
| AAG | K | 31.9 | 1295568 | AGG | R | 12 | 486463 |
| GAT | D | 21.8 | 885429 | GGT | G | 10.8 | 437126 |
| GAC | D | 25.1 | 1020595 | GGC | G | 22.2 | 903565 |
| GAA | E | 29 | 1177632 | GGA | G | 16.5 | 669873 |
| GAG | E | 39.6 | 1609975 | GGG | G | 16.5 | 669768 |

For example, the codons TCT, TCC, TCA, TCG, AGT and AGC all code for Serine (note that T in the DNA equivalent to the U in RNA). From a human codon usage frequency, such as set forth in Table 1 above, the corresponding usage frequencies for these codons are 15.2, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal in which the transgene is intended to be expressed.

In some embodiments, the nucleic acid sequence encoding GBA1 comprises the sequence set forth in SEQ ID NO:2 or a coding region of SEQ ID NO:2. In some embodiments, the nucleic acid sequence encoding GBA1 comprises a codon-optimized version of the sequence set forth in SEQ ID NO:2 or a coding region of SEQ ID NO:2. In some embodiments, the nucleic acid sequence encoding GBA1 comprises the sequence set forth in SEQ ID NO:6. In some embodiments, the nucleic acid sequence encoding GBA1 comprises a codon-optimized version of the sequence set forth in SEQ ID NO:2 or a coding region of SEQ ID NO:2 and a reporter sequence. In some embodiments, the nucleic acid sequence encoding GBA1 comprises the sequence set forth in SEQ ID NO:7.

In some embodiments, the transgene is positioned between AAV inverted terminal repeats (ITRs). In some embodiments, a nucleic acid sequence encoding GBA1 is positioned between AAV ITRs. In some embodiments, a nucleic acid sequence encoding an amino acid sequence comprising the amino acid sequence set forth in SEQ ID NO: 1 is positioned between AAV ITRs. In some embodiments, the nucleic acid sequence set forth in SEQ ID NO:2 or a coding region of SEQ ID NO:2, or a codon-optimized version thereof is positioned between AAV ITRs. In some embodiments, a coding region of the nucleic acid sequence set forth in SEQ ID NO:2 is positioned between AAV ITRs. In some embodiments, a codon-optimized version of a coding region of the nucleic acid sequence set forth in SEQ ID NO:2 is positioned between AAV ITRs. In some embodiments, the nucleic acid sequence set forth in SEQ ID NO: 6 is positioned between AAV ITRs. In some embodiments, the nucleic acid sequence set forth in SEQ ID NO:7 is positioned between AAV ITRs.

In some embodiments, the nucleic acid sequence encoding GBA1 is operably linked to a promoter (i.e., the nucleic acid sequence is under the control of the promoter). In some embodiments, the promoter is derived from actin genes, immunoglobulin genes, cytomegalovirus (CMV), ubiquitin C (UBC) promoter, adenovirus, bovine papilloma virus, adenoviral promoters, such as the adenoviral major late promoter, an inducible heat shock promoter, respiratory syncytial virus, or Rous sarcomas virus (RSV). In some embodiments, the promoter is a chicken beta actin promoter, a chicken beta actin promoter with a cytomegalovirus enhancer, or a modification thereof. In some embodiments, the promoter is a CMV promoter. In some embodiments, the promoter is a UBC promoter.

In some embodiments, a nucleic acid sequence encoding GBA1, operably linked to a promoter, is positioned between AAV ITRs. In some embodiments, a nucleic acid sequence encoding an amino acid sequence comprising the amino acid sequence set forth in SEQ ID NO:1, operably linked to a promoter, is positioned between AAV ITRs. In some embodiments, the nucleic acid sequence set forth in SEQ ID NO:2 or a coding region of SEQ ID NO:2, or a codon-optimized version thereof, operably linked to a promoter, is positioned between AAV ITRs. In some embodiments, a coding region of the nucleic acid sequence set forth in SEQ ID NO:2, operably linked to a promoter, is positioned between AAV ITRs. In some embodiments, a codon-optimized version of a coding region of the nucleic acid sequence set forth in SEQ ID NO:2, operably linked to a promoter, is positioned between AAV ITRs. In some embodiments, the nucleic acid sequence set forth in SEQ ID NO:6, operably linked to a promoter, is positioned between AAV ITRs. In some embodiments, the nucleic acid sequence set forth in SEQ ID NO: 7, operably linked to a promoter, is positioned between AAV ITRs.

### C. Delivery of AAV Vector

In some embodiments, the rAAV vector is purified, and the number of viral particles are quantified, prior to introduction into a cell. In some embodiments, the rAAV vector is introduced into a cell by transduction, such as by incubation of the rAAV particles with the cells in culture.

In some embodiments, the transducing comprises incubating viral particles of the rAAV vector with the cell in culture. In some embodiments, the incubating is carried out for about 8 hours, about 12 hours, about 16 hours, about 20 hours, or about 24 hours. In some embodiments, the incubating is carried out for about 8 hours. In some embodiments, the incubating is carried out for about 12 hours. In some embodiments, the incubating is carried out for about 16 hours. In some embodiments, the incubating is carried out for about 20 hours. In some embodiments, the incubating is carried out for about 24 hours.

In some embodiments, cells, such as neurally differentiated cells (e.g., day 16 cells), are transduced with between about 3,000 and 150,000 viral particles per cell of the rAAV vector. In some embodiments, neurally differentiated cells are transduced with between about 100 and 150,00 viral particles per cell of a rAAV9 vector. In some embodiments, neurally differentiated cells are transduced with between about 100 and 10,00 viral particles per cell of a rAAV9 vector. In some embodiments, neurally differentiated cells are transduced with between about 10,000 and 150,00 viral particles per cell of a rAAV9 vector. In some embodiments, neurally differentiated cells are transduced with between about 3,000 and 100,00 viral particles per cell of a rAAV7m8 vector. In some embodiments, neurally differentiated cells are transduced with between about 3,000 and 100,00 viral particles per cell of a rAAV-LK03 vector.

In some embodiments, prior to the introducing of the rAAV vector, the neurally differentiated cells have been cryopreserved. In some embodiments, the neurally differentiated cells were cryopreserved between about day 16 and about day 18. In some embodiments, the neurally differentiated cells were cryopreserved on about day 16. In some embodiments, the neurally differentiated cells were cryopreserved on about day 18. In some embodiments, the neurally differentiated cells were cryopreserved on about day 20. Thus, in some embodiments, prior to the introducing of the rAAV vector, the neurally differentiated cells are thawed. In some embodiments, the neurally differentiated cells are thawed and allowed to rest until the introducing the next day.

In some embodiments, the neurally differentiated cells are day 16 cells. In some embodiments, the neurally differentiated cells are day 17 cells. In some embodiments, the neurally differentiated cells are day 18 cells. In some embodiments, the neurally differentiated cells are day 19 cells. In some embodiments, the neurally differentiated cells are day 20 cells. In some embodiments, the cells are transduced on about day 16 and are further differentiated until harvest. In some embodiments, the cells are transduced on about day 18 and are further differentiated until harvest. In some embodiments, the cells are transduced on about day 20 and are further differentiated until harvest.

In some embodiments, the cells are harvested on about day 18. In some embodiments, the cells are harvested on about day 19. In some embodiments, the cells are harvested on about day 20. In some embodiments, the cells are harvested on about day 21. In some embodiments, the cells are harvested on about day 22. In some embodiments, the cells are harvested on about day 23.

### D. Selection of Modified Cells

In some embodiments, the cells that were introduced with a rAAV vector comprising a promoter operably linked to a nucleic acid sequence encoding GBA1 (i.e., a GBA1-containing transgene) in accordance with the methods herein, e.g., as described in Section III.B, are screened and/or selected for cells where the GBA1 expression is increased as compared to prior to the introducing. In some embodiments, the cells that were introduced with a rAAV vector comprising a promoter operably linked to a nucleic acid sequence encoding GBA1 (i.e., a GBA1-containing transgene) in accordance with the methods herein, e.g., as described in Section III.B, are screened and/or selected for cells where the GCase activity is increased as compared to prior to the introducing. In some embodiments, the cells are assessed to identify changes attributable to the methods described herein, e.g, as described in Section III.B, such as introduction of a rAAV vector into the cells.

In some embodiments, the assessment includes determining the expression of GBA1 in the cells introduced with the rAAV vector, such as by any methods known in the art. In particular embodiments, assessing, measuring, and/or determining gene expression is or includes determining or measuring the level, amount, or concentration of the gene product. In certain embodiments, the level, amount, or concentration of the gene product may be transformed (e.g., normalized) or directly analyzed (e.g., raw). In some embodiments, the gene product is a protein that is encoded by the gene. In certain embodiments, the gene product is a polynucleotide, e.g., an mRNA or a protein, that is encoded by the gene.

In particular embodiments, the amount or level of a polynucleotide in a sample may be assessed, measured, determined, and/or quantified by any suitable means known in the art. For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR (qPCR) methods; northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art.

In some embodiments, the assessment includes determining the protein level of the GCase enzyme, such as by any methods known in the art. Qualifying the level of the Gcase enzyme protein may be carried out by any suitable means known in the art. Suitable methods for assessing, measuring, determining, and/or quantifying the level, amount, or concentration or more or more protein gene products include, but are not limited to detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemisty or immunohistochemisty, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In some embodiments, the assessment includes determining the activity level of the GCase enzyme, such as by any methods known in the art. For example, in some embodiments, the activity level of the GCase enzyme is assessed by an enzymatic activity reaction wherein protein isolated from cells is combined with 4-methylumbelliferyl beta-D-glucopyranosidase (4-MBDG) substrate, and cleavage of the substrate by GCase yields 4-methylumbelliferone (4-MU), the concentration of which may be measured, such as by reference to standard or known value(s).

In some embodiments, it is desirable to increase the GCase activity in one or more cells (e.g., a clone) into which a nucleic acid sequence encoding GBA1 is introduced by between about 100% and about 200%, as compared to the GCase activity in the one or more cells prior to introduction of the nucleic acid sequence encoding GBA1. Thus, in some embodiments, one or more cells (e.g., a clone) is selected in which the introduction of a nucleic acid sequence encoding GBA1 has increased the GCase activity by between about 100% and about 200%. In some embodiments, one or more cells (e.g., a clone) is selected in which the introduction of GBA1 has increased its GCase activity by about 100%. In some embodiments, one or more cells (e.g., a clone) is selected in which the introduction of GBA1 has increased its GCase activity by about 120%. In some embodiments, one or more cells (e.g., a clone) is selected in which the introduction of GBA1 has increased its GCase activity by about 140%. In some embodiments, one or more cells (e.g., a clone) is selected in which the introduction of GBA1 has increased its GCase activity by about 160%. In some embodiments, one or more cells (e.g., a clone) is selected in which the introduction of GBA1 has increased its GCase activity by about 180%. In some embodiments, one or more cells (e.g., a clone) is selected in which the introduction of GBA1 has increased its GCase activity by about 200%. In some embodiments, the one or more cells (e.g., a clone) is selected for differentiation. In some embodiments, the one or more cells (e.g., a clone) is selected for use in treating a disease or condition.

### IV. Compositions and Formulations

Also provided herein are populations of overexpressing (i.e., GBA1 overexpressing) cells, compositions containing overexpressing cells, and compositions enriched for overexpressing cells. The overexpressing cells are cells, e.g., differentiated cells derived from PSCs, such as iPSCs, that have been introduced with a rAAV vector comprising a nucleic acid sequence encoding GBA1 by any of the methods described in Section III. In some embodiments, the overexpressing cells, the compositions containing overexpressing cells, and compositions enriched for overexpressing cells, are produced by the methods described herein, e.g., as described in Section II and Section III. In some embodiments, the population of overexpressing cells, the composition containing overexpressing cells, and the compositions enriched for overexpressing cells, include overexpressing cells that are differentiated neural cells, such as floor plate midbrain progenitor cells, determined dopamine (DA) neuron progenitor cells, and/or dopamine (DA) neurons, or glial cells, e.g., microglial cells, astrocytes, oligodendrocytes, or ependymocytes. In some embodiments, the population of overexpressing cells, the composition containing overexpressing cells, and the compositions enriched for overexpressing cells, include overexpressing cells that are macrophages. In some embodiments, the population of overexpressing cells, the composition containing overexpressing cells, and the compositions enriched for overexpressing cells, include overexpressing cells that are hematopoietic stem cells (HSCs). In some embodiments, the provided population of overexpressing cells is a population of the cell produced by any the methods described herein, e.g., as described in Section II and Section III. Accordingly, also provided herein is a population of the cell produced by any the methods described herein, e.g., as described in Section II and Section III, as well as compositions comprising the cell produced by any the methods described herein, e.g., as described in Section II and Section III, and compositions enriched for the cell produced by any the methods described herein, e.g., as described in Section II and Section III.

In some embodiments, the provided population of overexpressing cells, composition containing overexpressing cells, or composition enriched for overexpressing cells, include a cell population comprising cells that express (e.g., episomally expresses) one or more transgene(s) containing GBA1, wherein a gene variant of GBA1 is associated with decreased GCase activity. In some embodiments, the GBA1 is the wildtype form or a functional form or portion thereof. In some embodiments, the gene variant is associated with PD. In some embodiments, the gene variant is associated with GD. In some embodiments, the provided population of overexpressing cells, composition containing overexpressing cells, or composition enriched for overexpressing cells, include a cell population comprising cells that express (e.g. episomally express) one or more transgene(s) containing a wildtype version of a GBA1, wherein a gene variant of GBA1 is associated with PD, such as a gene variant associated with PD that is within the human GBA1 locus. In some embodiments, at least 75%, 80%, 85%, 90%, 95%, 96%, 97%, 98%, or 99%, or 100 of the cells in the population of overexpressing cells, composition containing overexpressing cells, or composition enriched for overexpressing cells have been engineered to express (e.g., episomally express) one or more transgene(s) containing GBA1. In some embodiments, at least 75%, 80%, 85%, 90%, 95%, 96%, 97%, 98%, or 99%, or 100 of the cells in the population of overexpressing cells, composition containing overexpressing cells, or composition enriched for overexpressing cells have been engineered to express (e.g., episomally express) one or more transgene(s) containing a wildtype version of GBA1. In some embodiments, the cells have been introduced with one or more transgene(s) containing GBA1 by the methods described herein. In some embodiments, the cells have been introduced with one or more transgene(s) containing a wildtype version of a GBA1 by the methods described herein. In some embodiments, the cells that have been introduced with the one or more transgene(s) containing GBA1 are less likely to cause, or contribute to, PD than the cells would be without the introducing. In some embodiments, the cells that have been introduced with the one or more transgene(s) containing a wildtype version of GBA1 are less likely to cause, or contribute to, PD than the cells would be without the introducing. In some embodiments, the cells that have been introduced with the one or more transgene(s) containing GBA1 are less likely to cause, or contribute to, GD than the cells would be without the introducing. In some embodiments, the cells that have been introduced with the one or more transgene(s) containing a wildtype version of GBA1 are less likely to cause, or contribute to, GD than the cells would be without the introducing. In some embodiments, the GBA1 is wildtype GBA1. In some embodiments, GBA1 is a functional GBA1 or a portion thereof.

### A. Exemplary Features of Compositions

In some embodiments, the cells produced by any of the methods described herein overexpress the GBA1 gene, such as compared to expression of the GBA1 gene in cells not produced by the methods described herein (i.e., cells not introduced with the rAAV vector). In some embodiments, at least 10%, at least 20%, at least 30%, at least 40%, or at least 50% of the cells of any of the compositions described herein overexpress the GBA1 gene. In some embodiments, at least 10% of the cells of any of the compositions described herein overexpress the GBA1 gene. In some embodiments, at least 20% of the cells of any of the compositions described herein overexpress the GBA1 gene. In some embodiments, at least 30% of the cells of any of the compositions described herein overexpress the GBA1 gene. In some embodiments, at least 40% of the cells of any of the compositions described herein overexpress the GBA1 gene. In some embodiments, at least 50% of the cells of any of the compositions described herein overexpress the GBA1 gene. In some embodiments, at least 60% of the cells of any of the compositions described herein overexpress the GBA1 gene. In some embodiments, at least 70% of the cells of any of the compositions described herein overexpress the GBA1 gene. In some embodiments, at least 80% of the cells of any of the compositions described herein overexpress the GBA1 gene. In some embodiments, at least 90% of the cells of any of the compositions described herein overexpress the GBA1 gene.

In some embodiments, GBA1 is wildtype GBA1. In some embodiments, GBA1 is a functional GBA1 or a portion thereof. In some embodiments, GBA1 is a functional GBA1.

In some embodiments, the differentiated cells produced by any of the methods described herein are determined dopaminergic (DA) neuron progenitor cells. In some embodiments, the determined DA neuron progenitor cells are introduced with a recombinant adeno-associated viral (rAAV) vector comprising a promoter operably linked to a nucleic acid sequence encoding GBA1 (i.e., a GBA1-containing transgene). In some embodiments the determined DA neuron progenitor cells introduced with the rAAV vector overexpress GBA1, such as compared to expression of the GBA1 gene in cells not introduced with the rAAV vector.

In some embodiments, the differentiated and/or the overexpressing cells comprise a variant of human GBA1. In some embodiments, the variant is a single nucleotide polymorphism (SNP). In some embodiments, the differentiated cells comprise a variant of human GBA1. In some embodiments, the overexpressing cells comprise a variant of human GBA1. In some embodiments, the variant is a single nucleotide polymorphism (SNP). In some embodiments, the SNP is rs76763715 (e.g., with reference to SEQ ID NO:2). In some of any such embodiments, the rs76763715 is a cytosine variant. In some of any such embodiments, the GBA1 comprising the SNP encodes a serine, rather than an asparagine, at amino acid position 370 (N370S) (e.g., with reference to SEQ ID NO:1). In some of any such embodiments, the SNP is rs421016 (e.g., with reference to SEQ ID NO:2). In some of any such embodiments, the rs421016 is a guanine variant. In some of any such embodiments, the GBA1 comprising the SNP encodes a proline, rather than a leucine, at amino acid position 444 (L444P) (e.g., with reference to SEQ ID NO:1). In some of any such embodiments, the SNP is rs2230288 (e.g., with reference to SEQ ID NO:2). In some of any such embodiments, the rs2230288 is a thymine variant. In some of any such embodiments, the GBA1 comprising the SNP encodes a lysine, rather than a glutamic acid, at position 326 (E326K) (e.g., with reference to SEQ ID NO:1).

In some embodiments, the differentiated cells produced by any of the methods described herein are capable of producing dopamine (DA). In some embodiments, the differentiated cells produced by any of the methods described herein do not produce or do not substantially produce norepinephrine (NE). Thus, in some embodiments, the differentiated cells produced by any of the methods described herein are capable of producing DA but do not produce or do not substantially produce NE.

In some embodiments, the determined dopamine (DA) neuron progenitor cells express EN1. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1.

In some embodiments, the determined dopamine (DA) neuron progenitor cells express CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express CORIN.

In some embodiments, the determined dopamine (DA) neuron progenitor cells express EN1 and CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1 and CORIN.

In some embodiments, less than 10% of determined dopamine (DA) neuron progenitor cells express TH. In some embodiments, the determined dopamine (DA) neuron progenitor cells express low levels of TH. In some embodiments, the determined dopamine (DA) neuron progenitor cells do not express TH. In some embodiments, the determined dopamine (DA) neuron progenitor cells express TH at lower levels than cells harvested or collected on other days. In some embodiments, some of the determined dopamine (DA) neuron progenitor cells express EN1 and CORIN and less than 10% of the cells express TH. In some embodiments, less than 10% of the determined dopamine (DA) neuron progenitor cells express TH, and at least about 20% of the cells express EN1. In some embodiments, less than 10% of the determined dopamine (DA) neuron progenitor cells express TH, and at least about 20% of the cells express CORIN. In some embodiments, less than 10% of the total determined dopamine (DA) neuron progenitor cells express TH, and at least about 20% of the cells express EN1 and CORIN.

In some embodiments, the differentiated cells produced by any of the methods described herein are dopamine (DA) neurons (e.g., midbrain fate DA neurons). In some embodiments, the midbrain fate dopamine (DA) neurons are FOXA2+/TH+ at the time of harvest. In some embodiments, the midbrain fate dopamine (DA) neurons are FOXA2+/TH+ by or on about day 18. In some embodiments, the midbrain fate dopamine (DA) neurons are FOXA2+/TH+ by or on about day 20. In some embodiments, the midbrain fate dopamine (DA) neurons are FOXA2+/TH+ by or on about day 25.

### B. Compositions and Formulations

In some embodiments, the dose of cells comprising cells produced by any of the methods disclosed herein, is provided as a composition or formulation, such as a pharmaceutical composition or formulation. In some embodiments, the dose of cells comprises differentiated cells introduced with a rAAV vector to overexpress GBA1. In some embodiments, GBA1 is a functional GBA1 or a portion thereof. In some embodiments, GBA1 is a functional GBA1. In some embodiments, GBA1 is wildtype GBA1. In some embodiments, the dose of cells comprises differentiated cells introduced with a rAAV vector to overexpress a wildtype form of GBA1. Thus, in some embodiments, the dose of cells comprises overexpressing cells. In some embodiments, the dose of cells comprises cells produced by any of the methods described in Section III. In some embodiments, the dose of cells comprises cells produced by a combination of (1) any of the methods described in Section II, and (2) any of the methods described in Section III. In some embodiments, the dose of cells comprises cells produced by a process comprising (1) any of the methods for differentiating cells described in Section II; and (2) any of the methods of introducing one or more rAAV vector(s) comprising a promoter operably linked to a nucleic acid sequence encoding GBA1 (i.e., a GBA1-containing transgene) described in Section III.

Such compositions can be used in accord with the provided methods, articles of manufacture, and/or with the provided compositions, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum. In some embodiments, the agents or cells can be administered by stereotactic injection into the striatum, such as in the putamen.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g., the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes

### V. Methods of Treatment

The present disclosure relates to methods of increasing the activity of GCase and/or increasing the expression of the wildtype form of GBA1, such as in a subject having decreased expression and/or a variant of GBA1 associated with Parkinson's Disease (PD), and methods of lineage specific differentiation of pluripotent stem cells (PSCs), including embryonic stem (ES) cells and induced pluripotent stem cells (iPSCs) into DA neuron progenitor cells, including those in which activity of GCase and/or expression of the wildtype form of GBA1 has been increased, for use in treating neurodegenerative diseases. Specifically, the methods, compositions, and uses thereof provided herein contemplate differentiation of pluripotent stem cells into DA neuron progenitors cells and increased activity or GCase and/or increased expression of GBA1. In some embodiments, the cells have one or more GBA1 variant(s) associated with GD and/or PD, e.g., as described in Section II. Thus, in some embodiments, the methods, compositions, and uses thereof provided herein contemplate differentiation of pluripotent stem cells into DA neuron progenitors cells and increased activity or GCase and/or increased expression of the wildtype form of GBA1, wherein one or more GBA1 variants is associated with PD, e.g., as described in Section III, for administration to subjects exhibiting a loss of a certain type of neuron, e.g., dopamine (DA) neurons, including Parkinson's disease. The methods, compositions, and uses thereof provided herein contemplate differentiation of pluripotent stem cells into DA neuron progenitors cells and increased activity of GCase and/or increase expression of the wildtype form of GBA1, wherein one or more GBA1 variants is associated with PD, e.g., as described in Section III, for administration to subjects exhibiting the one or more GBA1 variants associated with PD. In some embodiments, the method increases the activity of GCase. In some embodiments, the method increases the expression of GBA1.

Specifically, provided herein is a method of treatment, comprising administering to a subject a therapeutically effective amount of a therapeutic composition, e.g., any composition as described in Section IV, wherein cells of the subject exhibit reduced activity of GCase and/or decreased expression of GBA1, as compared to reference cells (e.g., cells of a subject without Parkinson's Disease). In some embodiments, prior to the administering, cells of the subject exhibit reduced activity of GCase. In some embodiments, prior to the administering, cells of the subject exhibit decreased expression of GBA1. In some embodiments, prior to the administering, cells of the subject exhibit reduced activity of GCase and decreased expression of GBA1. In some embodiments, the reference cells do not exhibit reduced GCase activity. In some embodiments, the references cells do not exhibit reduced GBA1 expression. In some embodiments, the references cells do not exhibit reduced GCase activity or GBA1 expression. In some embodiments, the reference cells are from a subject without a LBD. In some embodiments, the reference cells are from a subject without PD. In some embodiments, the reference cells are from a subject without GD. In some embodiments, the reference cells are from a subject without PD or GD.

Also provided herein is a method of treatment, comprising administering to a subject a therapeutically effective amount of a therapeutic composition, e.g., any composition as described in Section IV, wherein the subject has reduced activity of the GCase enzyme.

Also provided herein is a method of treatment, comprising administering to a subject a therapeutically effective amount of a therapeutic composition, e.g., any composition as described in Section IV, wherein the subject has a gene variant, e.g., SNP, associated with PD, such as a gene variant in human GBA1. In some embodiments, the subject has PD. In some embodiments, the subject has GD.

In some embodiments, the subject has a gene variant in the GBA1 gene, e.g., a rs76763715 SNP, that results in an N370S amino acid change due to the presence of a serine, rather than an asparagine, at amino acid position 370 in the expressed GCase enzyme (e.g., with reference to SEQ ID NO: 1). In some embodiments, the subject has a gene variant in the GBA1 gene, e.g., a rs421016 SNP, that results in an L444P amino acid change due to the presence of a proline, rather than a leucine, at position 444 in the expressed GCase enzyme (e.g., with reference to SEQ ID NO:1). In some embodiments, the subject has a gene variant in the GBA1 gene, e.g., a rs2230288 SNP, that results in an E326K amino acid change due to the presence of a lysine, rather than a glutamic acid, at position 326 in the expressed GCase enzyme (e.g., with reference to SEQ ID NO:1).

Also provided herein is a method of treatment, comprising administering to a subject a therapeutically effective amount of a therapeutic composition, e.g., any composition as described in Section IV, wherein the subject has one or more gene variant(s), e.g., SNP, associated with GD, such as a gene variant in human GBA1. In some embodiments, the subject has a homozygous mutation (variant) in GBA1. In some embodiments, the subject has biallelic mutatations in GBA1 (i.e., the mutations in each allele are not necessarily the same).

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is a Lewy body disease (LBD). In some embodiments, such as Parkinson's disease, Parkinson's disease dementia, or dementia with Lewy bodies (DLB). In some embodiments, the neurodegenerative disease is Parkinsonism. In some embodiments, the neurodegenerative disease is Parkinson's disease dementia. In some embodiments, the neurodegenerative disease is DLB. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has Parkinson's Disease. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, cells of the subject have a GBA1 gene that includes a gene variant associated with PD. In some embodiments, the GBA1 variant encodes a serine, rather than an asparagine, at position 370 (N370S) (e.g., with reference to SEQ ID NO:1). In some embodiments, the GBA1 variant encodes an amino acid sequence comprising the amino acid sequence set forth in SEQ ID NO: 3. In some embodiments, the GBA1 variant encodes a proline, rather than a leucine, at position 444 (L444P) (e.g., with reference to SEQ ID NO:1). In some embodiments, the GBA1 variant encodes an amino acid sequence comprising the amino acid sequence set forth in SEQ ID NO: 4. In some embodiments, the GBA1 variant encodes a lysine, rather than a glutamic acid, at position 326 (E326K) (e.g., with reference to SEQ ID NO:1). In some embodiments, the GBA1 variant encodes an amino acid sequence comprising the amino acid sequence set forth in SEQ ID NO: 5. In some embodiments, the GBA1 variant encodes an amino acid sequence comprising the amino acid sequence set forth in any one of SEQ ID NOs: 3, 4, and 5.

In some embodiments, the subject has a GBA1 variant associated with PD that is a variant of rs76763715. In some embodiments, the subject has a GBA1 variant associated with PD that is a variant of rs76763715 that encodes a serine, rather than an asparagine, at position 370 (N370S). In some embodiments, the subject has a GBA1 variant associated with PD that is a a cytosine variant of rs76763715.

In some embodiments, the subject has a GBA1 variant associated with PD that is a variant of rs421016. In some embodiments, the subject has a GBA1 variant associated with PD that is a variant of rs421016 that encodes a proline, rather than a leucine, at position 444 (L444P). In some embodiments, the subject has a GBA1e variant associated with PD that is a guanine variant of rs421016.

In some embodiments, the subject has a GBA1 variant associated with PD that is a variant of rs2230288. In some embodiments, the subject has a GBA1 variant associated with PD that is a variant of rs2230288 that encodes a lysine, rather than a glutamic acid, at position 326 (E326K). In some embodiments, the subject has a GBA1 variant associated with PD that is a thymine variant of rs2230288.

In some embodiments, cells of the subject have a GBA1 gene that includes a gene variant associated with PD. In some embodiments, the GBA1 variant encodes the amino acid sequence set forth in any one of SEQ ID NOS: 8-17. In some embodiments, the GBA1 variant encodes a methionine, rather than a threonine, at position 369 (T369M) (e.g., with reference to SEQ ID NO:1). In some embodiments, the GBA1 variant encodes an amino acid sequence comprising the amino acid sequence set forth in SEQ ID NO: 8. In some embodiments, the GBA1 variant encodes a serine, rather than a glycine, at position 377 (G377S) (e.g., with reference to SEQ ID NO:1). In some embodiments, the GBA1 variant encodes an amino acid sequence comprising the amino acid sequence set forth in SEQ ID NO: 9. In some embodiments, the GBA1 variant encodes a histidine, rather than an aspartic acid, at position 409 (D409H) (e.g., with reference to SEQ ID NO:1). In some embodiments, the GBA1 variant encodes an amino acid sequence comprising the amino acid sequence set forth in SEQ ID NO: 10. In some embodiments, the GBA1 variant encodes a tryptophan, rather than an arginine, at position 120 (R120W) (e.g., with reference to SEQ ID NO:1). In some embodiments, the GBA1 variant encodes an amino acid sequence comprising the amino acid sequence set forth in SEQ ID NO: 11. In some embodiments, the GBA1 variant encodes a leucine, rather than a valine, at position 394 (V394L) (e.g., with reference to SEQ ID NO:1). In some embodiments, the GBA1 variant encodes an amino acid sequence comprising the amino acid sequence set forth in SEQ ID NO: 12. In some embodiments, the GBA1 variant encodes a histidine, rather than an arginine at position 496 (R496H) (e.g., with reference to SEQ ID NO:1). In some embodiments, the GBA1 variant encodes an amino acid sequence comprising the amino acid sequence set forth in SEQ ID NO: 13. In some embodiments, the GBA1 variant encodes a threonine, rather than a lysine, at position 178 (K178T) (e.g., with reference to SEQ ID NO:1). In some embodiments, the GBA1 variant encodes an amino acid sequence comprising the amino acid sequence set forth in SEQ ID NO: 14. In some embodiments, the GBA1 variant encodes a cysteine, rather than an arginine, at position 329 (R329C) (e.g., with reference to SEQ ID NO:1). In some embodiments, the GBA1 variant encodes an amino acid sequence comprising the amino acid sequence set forth in SEQ ID NO: 15. In some embodiments, the GBA1 variant encodes an arginine, rather than a leucine, at position 444 (L444R) (e.g., with reference to SEQ ID NO:1). In some embodiments, the GBA1 variant encodes an amino acid sequence comprising the amino acid sequence set forth in SEQ ID NO: 16. In some embodiments, the GBA1 variant encodes a serine, rather than an asparagine, at position 188 (N188S) (e.g., with reference to SEQ ID NO:1). In some embodiments, the GBA1 variant encodes an amino acid sequence comprising the amino acid sequence set forth in SEQ ID NO: 17.

In some embodiments, the therapeutic composition comprising cells, e.g., differentiated cells, having increased expression of GBA1 (overexpressing cells), is administered to treat a subject having a disease or disorder associated with reduced GCase activity. In some embodiments, the therapeutic composition comprising cells, e.g., differentiated cells, having increased expression of the wildtype form of the GBA1 (overexpressing cells), is administered to treat a neurodegenerative disease. In some embodiments, the neurodegenerative disease is a LBD. In some embodiments, the neurodegenerative disease is Parkinson's disease dementia. In some embodiments, the neurodegenerative disease is DLB. In some embodiments, the neurodegenerative disease is PD. In some embodiments, the neurodegenerative disease is GD. In some embodiments, the therapeutic composition comprising cells, e.g., differentiated cells, having increased expression of the wildtype form of the GBA1 (overexpressing cells), is administered to treat a neurodegenerative disease, e.g., PD, using cells that exhibit increased expression of (i.e., overexpress) the wildtype form of GBA1. By administering a therapeutic composition comprising cells, e.g., differentiated cells, exhibiting increased expression of (i.e., overexpressing) GBA1, the risk of recurrence of the neurodegenerative disease, e.g., PD, is reduced.

In some embodiments, a dose of cells overexpressing GBA1, e.g., as described in Section III, that have been neurally differentiated, e.g., as described in Section II, is administered to subjects in accord with the provided methods, and/or with the provided articles of manufacture, and/or with the provided compositions, e.g., as described in Section IV. The dose of cells is a dose of cells, e.g., DA neuron progenitor cells, overexpressing GBA1, e.g., as described in Section III, that have been differentiated from pluripotent stem cells, e.g., as described in Section II. In some embodiments, the dose of cells is a dose of a composition of cells, e.g., as described in Section IV.

In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum (e.g., putamen) of the subject. In some embodiments, the dose of cells is administered to one hemisphere of the subject's striatum (e.g., putamen). In some embodiments, the dose of cells is administered to both hemispheres of the subject's striatum (e.g., putamen).

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about about 3×10⁶ cells/hemisphere and 15×10⁶ cells/hemisphere. In some embodiments, the dose of cells is about about 3×10⁶ cells/hemisphere. In some embodiments, the dose of cells is about about 4×10⁶ cells/hemisphere. In some embodiments, the dose of cells is about about 5×10⁶ cells/hemisphere. In some embodiments, the dose of cells is about about 6×10⁶ cells/hemisphere. In some embodiments, the dose of cells is about about 7×10⁶ cells/hemisphere. In some embodiments, the dose of cells is about about 8×10⁶ cells/hemisphere. In some embodiments, the dose of cells is about about 9×10⁶ cells/hemisphere. In some embodiments, the dose of cells is about about 10×10⁶ cells/hemisphere. In some embodiments, the dose of cells is about about 11×10⁶ cells/hemisphere. In some embodiments, the dose of cells is about about 12×10⁶ cells/hemisphere. In some embodiments, the dose of cells is about about 13×10⁶ cells/hemisphere. In some embodiments, the dose of cells is about about 14×10⁶ cells/hemisphere. In some embodiments, the dose of cells is about about 15×10⁶ cells/hemisphere.

In some embodiments, the dose of cells is about about 5×10⁶ cells in each putamen. In some embodiments, the dose of cells is about about 10×10⁶ cells in each putamen.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g., overexpressing cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-types In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some aspects, the size of the dose is determined based on one or more criteria such as response of the subject to prior treatment, e.g., disease type and/or stage, and/or likelihood or incidence of the subject developing toxic outcomes, e.g., dyskinesia.

### VI. Articles of Manufacture and Kits

Also provided are articles of manufacture, systems, apparatuses, and kits useful in performing the provided methods.

Also provided are articles of manufacture, including: (i) a recombinant adeno-associated viral (rAAV) vector comprising a promoter operably linked to a nucleic acid sequence encoding GBA1; and (ii) instructions for use of the rAAV vector for performing any methods described herein.

In some of any such embodiments, the rAAV vector is pseudotyped with capsid proteins of an AAV serotype. In some embodiments, the capsid proteins are of the AAV2, AAV3, AAV3B, AAV3H, AAVLK-03, AAV5, AAV6, AAV7m8, AAV8, or AAV9 serotype, optionally of the AAV7m8, AAV9, or AAV-LK03 serotype. In some embodiments, the capsid proteins are of the AAV9 serotype. In some embodiments, the capsid proteins are of the AAV7m8 serotype. In some embodiments, the capsid proteins are of the AAV-LK03 serotype.

In some embodiments, the nucleic acid sequence encoding GBA1 is positioned between inverted terminal repeat (ITRs). In some embodiments, the ITRs are of the same serotype as the capsid proteins.

In some embodiments, the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter. In some embodiments, the promoter is a UBC promoter.

Also provided are articles of manufacture, including: (i) one or more reagents for differentiation of pluripotent stem cells into floor plate midbrain progenitor cells, determined dopaminergic (DA) neuron progenitor cells, and/or dopaminergic (DA) neurons; and (ii) instructions for use of the one or more reagents for performing any methods described herein.

Also provided are articles of manufacture, including: (i) a recombinant adeno-associated viral (rAAV) vector comprising a promoter operably linked to a nucleic acid sequence encoding GBA1; (ii) one or more reagents for differentiation of pluripotent stem cells into floor plate midbrain progenitor cells, determined dopamine (DA) neuron progenitor cells, and/or dopamine (DA) neurons; and instructions for use of the rAAV vector and the one or more reagents for performing any methods described herein.

In some of any such embodiments, the reagent for differentiation is or includes a small molecule, capable of inhibiting TGF-β/activin-Nodal signaling. In some of any such embodiments, the reagent for differentiation is or includes SB431542. In some of any such embodiments, the reagent for differentiation is or includes a small molecule, capable of activating SHH signaling. In some of any such embodiments, the reagent for activating SHH signaling is or includes SHH. In some of any such embodiments, the reagent for activating SHH signaling is or includes purmorphamine. In some of any such embodiments, the reagent for activating SHH signaling is or includes SHH and purmorphamine. In some of any such embodiments, the reagent for differentiation is or includes a small molecule, capable of inhibiting BMP signaling. In some of any such embodiments, the reagent for inhibiting BMP signaling is LDN193189. In some of any such embodiments, the reagent for differentiation is or includes a small molecule, capable of inhibiting GSK3B signaling. In some of any of such embodiments, the reagent is or includes CHIR99021. In some of any of such embodiments, the reagent for differentiation is or includes one or more of BDNF, GDNF, dbcAMP, ascorbic acid, TGFβ3, and DAPT. The reagents in the kit in one embodiment may be in solution, may be frozen, or may be lyophilized.

Also provided are articles of manufacture, including (i) any composition described herein; and (ii) instructions for administering the composition to a subject.

In some embodiments, the articles of manufacture or kits include one or more containers, typically a plurality of containers, packaging material, and a label or package insert on or associated with the container or containers and/or packaging, generally including instructions for use, e.g., instructions for reagents for differentiation of pluripotent cells, e.g., differentiation of iPSCs into floor plate midbrain progenitor cells, determined dopamine (DA) neuron progenitor cells, and/or dopamine (DA) neurons, and instructions to carry out any of the methods provided herein. In some aspects, the provided articles of manufacture contain reagents for differentiation and/or maturation of cells, for example, at one or more steps of the manufacturing process, such as any reagents described in any steps of Sections II and IV.

Also provided are articles of manufacture and kits containing overexpressing and differentiated cells, such as those generated using the methods provided herein, and optionally instructions for use, for example, instructions for administering. In some embodiments, the instructions provide directions or specify methods for assessing if a subject, prior to receiving a cell therapy, is likely or suspected of being likely to respond and/or the degree or level of response following administration of differentiated cells for treating a disease or disorder. In some aspects, the articles of manufacture can contain a dose or a composition of overexpressing and differentiated cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the reagents and/or cell compositions are packaged separately. In some embodiments, each container can have a single compartment. In some embodiments, other components of the articles of manufacture or kits are packaged separately, or together in a single compartment.

### VII. Exemplary Embodiments

Among the provided embodiments are:

1. A method of increasing expression of GBA1 in a cell, the method comprising:
introducing, into a neurally differentiated cell, a recombinant adeno-associated viral (rAAV) vector comprising a promoter operably linked to a nucleic acid sequence encoding GBA1,
wherein the introducing results in increased expression of GBA1 in the cell.

2. The method of embodiment 1, wherein the cell comprises a variant of GBA1 associated with Parkinson's disease.

3. A method of increasing expression of GBA1 in a cell, the method comprising:
introducing, into a neurally differentiated cell, a recombinant adeno-associated viral (rAAV) vector comprising a promoter operably linked to a nucleic acid sequence encoding GBA1,
wherein the cell comprises a variant of GBA1 associated with Parkinson's disease, and the introducing results in increased expression of GBA1 in the cell.

4. The method of any one of embodiments 1-3 wherein the rAAV vector is pseudotyped with capsid proteins of an AAV serotype.

5. The method of any one of embodiment 1-4, wherein the capsid proteins are of the AAV2, AAV3, AAV3B, AAV3H, AAVLK-03, AAV5, AAV6, AAV7m8, AAV8, or AAV9 serotype, optionally of the AAV7m8, AAV9, or AAV-LK03 serotype.

6. The method of any one of embodiments 1-5, wherein the capsid proteins are of the AAV9 serotype.

7. The method of any one of embodiments 1-6, wherein the nucleic acid sequence encoding GBA1 is positioned between inverted terminal repeat (ITRs).

8. The method of embodiment 7, wherein the ITRs are of the same serotype as the capsid proteins.

9. The method of any one of embodiments 1-8, wherein the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter.

10. The method of any one of embodiments 1-9, wherein the promoter is a UBC promoter.

11. The method of any one of embodiments 1-10, wherein the cell exhibits decreased expression of GBA1, as compared to a reference cell, optionally as compared to a cell from a subject without Parkinson's disease.

12. The method of any one of embodiments 1-11, wherein the cell exhibits reduced activity of the β-Glucocerebrosidase (GCase) enzyme encoded by GBA1 prior to being introduced with the rAAV vector, as compared to a reference cell, optionally as compared to a cell from a subject without Parkinson's disease.

13. The method of any one of embodiments 1-12, wherein GBA1 is human GBA1.

14. The method of any one of embodiments 1-13, wherein the nucleic acid sequence comprises the sequence set forth in SEQ ID NO:6 or SEQ ID NO:7.

15. The method of any one of embodiments 1-14, wherein the nucleic acid sequence encodes an amino acid sequence comprising the amino acid sequence set forth in SEQ ID NO:1.

16. The method of any one of embodiments 2-15, wherein the variant of GBA1 comprises a single nucleotide polymorphism (SNP) that is associated with Parkinson's disease.

17. The method of embodiment 16, wherein the SNP is rs76763715.

18. The method of embodiment 17, wherein the rs76763715 is a cytosine variant.

19. The method of any one of embodiments 16-18, wherein the variant of GBA1 comprising a SNP encodes a serine, rather than an asparagine, at amino acid position 370 (N370S).

20. The method of embodiment 18 or embodiment 19, wherein the wild-type form of GBA1 comprises a thymine instead of the cytosine variant.

21. The method of embodiment 16, wherein the SNP is rs421016.

22. The method of embodiment 21, wherein the rs421016 is a guanine variant.

23. The method of any one of embodiments 16, 21, and 22, wherein the variant of GBA1 comprising the SNP encodes a proline, rather than a leucine, at amino acid position 444 (L444P).

24. The method of embodiment 22 or embodiment 23, wherein the wild-type form of GBA1 comprises an adenine instead of the guanine variant.

25. The method of embodiment 16, wherein the SNP is rs2230288.

26. The method of embodiment 25, wherein the rs2230288 is a thymine variant.

27. The method of any one of embodiments 16, 25, and 26, wherein the variant of GBA1 comprising the SNP encodes a lysine, rather than a glutamic acid, at position 326 (E326K).

28. The method of embodiment 26 or embodiment 27, wherein the wild-type form of GBA1 comprises a cytosine instead of the thymine variant.

29. The method of any one of embodiments 1-28, wherein the neurally differentiated cell is a dopaminergic neuron progenitor cell or a dopaminergic neuron.

30. The method of any one of embodiments 1-29, wherein the cell, optionally the dopaminergic neuron progenitor cell, is derived from a pluripotent stem cell (PSC), optionally an induced pluripotent stem cell (iPSC).

31. The method of any one of embodiments 1-30, wherein a plurality of the neurally differentiated cells, optionally the dopaminergic neuron progenitor cells, were differentiated from pluripotent stem cells (PSCs), optionally induced pluripotent stem cells (iPSCs), by a method comprising:
(a) performing a first incubation comprising culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

32. A method of differentiating neural cells, the method comprising:
(a) performing a first incubation comprising culturing pluripotent stem cells (PSCs) in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling;
(b) performing a second incubation comprising culturing cells of the spheroid in a substrate-coated culture vessel under conditions to neurally differentiate the cells; and
(c) introducing into the neurally differentiated cells a recombinant adeno-associated viral (rAAV) vector comprising a promoter operably linked to a nucleic acid sequence encoding GBA1, wherein the introducing results in increased expression of GBA1 in the cell.

33. The method of embodiment 32, wherein the cells comprise a variant of GBA1 associated with Parkinson's disease.

34. The method of embodiment 32 or embodiment 33, wherein the cells exhibit decreased expression of GBA1, as compared to a reference cell, optionally as compared to a cell from a subject without Parkinson's disease.

35. The method of any one of embodiments 32-34, wherein the cells exhibit reduced activity of the β-Glucocerebrosidase (GCase) enzyme encoded by GBA1 prior to being introduced with the rAAV vector, as compared to a reference cell, optionally as compared to a cell from a subject without Parkinson's disease.

36. The method of any one of embodiments 31-35, wherein the cells are exposed to the inhibitor of TGF-β/activin-Nodal signaling up to a day at or before day 7.

37. The method of any one of embodiments 31-36, wherein the cells are exposed to the inhibitor of TGF-β/activin-Nodal beginning at day 0 and through day 6, inclusive of each day.

38. The method of any one of embodiments 31-37, wherein the cells are exposed to the at least one activator of SHH signaling up to a day at or before day 7.

39. The method of any one of embodiments 31-38, wherein the cells are exposed to the at least one activator of SHH signaling beginning at day 0 and through day 6, inclusive of each day.

40. The method of any one of embodiments 31-39, wherein the cells are exposed to the inhibitor of BMP signaling up to a day at or before day 11.

41. The method of any one of embodiments 31-40, wherein the cells are exposed to the inhibitor of BMP signaling beginning at day 0 and through day 10, inclusive of each day.

42. The method of any one of embodiments 31-41, wherein the cells are exposed to the inhibitor of GSK3β signaling up to a day at or before day 13.

43. The method of any one of embodiments 31-42, wherein the cells are exposed to the inhibitor of GSK3β signaling beginning at day 0 and through day 12, inclusive of each day.

44. The method of any one of embodiments 31-43, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

45. The method of embodiment 44, wherein the cells are exposed to BAGCT and the inhibitor of Notch signaling beginning on day 11.

46. The method of embodiment 44 or embodiment 45, wherein the cells are exposed to BAGCT and the inhibitor of Notch signaling beginning at day 11 and until harvest of the neurally differentiated cells, optionally until day 18, optionally until day 20, or optionally until day 25.

47. The method of any one of embodiments 31-46, wherein the inhibitor of TGF-β/activin-Nodal signaling is SB431542.

48. The method of any one of embodiments 31-47, wherein the at least one activator of SHH signaling is SHH or purmorphamine.

49. The method of any one of embodiments 31-48, wherein the inhibitor of BMP signaling is LDN193189.

50. The method of any one of embodiments 31-49, wherein the inhibitor of GSK3β signaling is CHIR99021.

51. The method of any one of embodiments 31-50, wherein the cells are introduced with the rAAV vector between about day 14 and about day 20, optionally on day 16 or day 18.

52. The method of any one of embodiments 31-51, wherein the cells are introduced with the rAAV vector on about day 16.

53. The method of any one of embodiments 31-52, wherein the cells are harvested between about day 18 and about day 25, optionally at day 18 or day 20.

54. The method of any one of embodiments 31-53, wherein the cells are harvested on about day 20.

55. The method of any one of embodiments 31-40, wherein the neurally differentiated cell was cryopreserved and subsequently thawed prior to the introducing.

56. The method of any one of embodiments 31-55, wherein the neurally differentiated cell was thawed about 1 day prior to the introducing.

57. The method of any one of embodiments 31-56, further comprising cryopreserving the neurally differentiated cell prior to the introducing.

58. The method of embodiment 57, wherein the cryopreserving comprises formulating the neurally differentiated cell with a cryoprotectant.

59. The method of any one of embodiments 45-58, wherein the PSC is an induced pluripotent stem cell (iPSC) derived from a non-pluripotent cell from a subject.

60. The method of embodiment 59, wherein the non-pluripotent cell is a fibroblast.

61. The method of embodiment 59 or embodiment 60, wherein the subject has Parkinson's disease or Gaucher's disease.

62. The method of any of embodiments 59-61, wherein the subject has Parkinson's disease.

63. A cell produced by the method of any one of embodiments 1-62.

64. A modified neurally differentiated cell comprising an exogenous deoxyribonucleic acid

(DNA) sequence encoding GBA1, wherein the DNA sequence is episomal in the cell.

65. The neurally differentiated cell of embodiment 64, wherein the DNA is encoded by an episomal vector.

66. The neurally differentiated cell of embodiment 64 or embodiment 65, wherein the episomal vector is an AAV vector.

67. The neurally differentiated cell of any one of embodiments 64-66, wherein the cell comprises a variant of GBA1 associated with Parkinson's disease.

68. The cell of any one of embodiments 63-67, wherein the cell expresses EN1 and CORIN.

69. The cell of any one of embodiments 63-68, wherein the cell is a committed dopaminergic precursor cell. 70. The cell of any one of embodiments 63-69, wherein the cell is formulated with a cryoprotectant.

71. A therapeutic composition comprising the cell of any of embodiments 63-70.

72. The therapeutic composition of embodiment 71, wherein cells of the composition express EN1 and CORIN and less than 10% of the total cells in the composition express TH.

73. The therapeutic composition of embodiment 71 or embodiment 72, wherein less than 5% of the total cells in the composition express TH.

74. The therapeutic composition of any one of embodiments 71-73, further comprising a cryoprotectant. 75. A method of treatment, comprising administering to a subject the therapeutic composition of any one of embodiments 71-74.

76. The method of embodiment 75, wherein the cells of the therapeutic composition are autologous to the subject.

77. The method of embodiment 75 or embodiment 76, wherein the subject has a disease or disorder associated with reduced GCase activity.

78. The method of any one of embodiments 75-77, wherein the subject has Gaucher's disease.

79. The method of any one of embodiments 75-77, wherein the subject has a Lewy body disease (LBD).

80. The method of embodiment 79, wherein the LBD is Parkinson's disease, Parkinson's disease dementia, or dementia with Lewy bodies (DLB).

81. The method of any one of embodiments 75-77, 79, and 80, wherein the subject has Parkinson's disease.

82. The method of any one of embodiments 75-81, wherein the administering comprises delivering the cells of the therapeutic composition by stereotactic injection.

83. The method of any one of embodiments 75-82, wherein the administering comprises delivering the cells of the therapeutic composition through a catheter.

84. The method of embodiment 82 or embodiment 83, wherein the cells of the therapeutic composition are delivered to the striatum of the subject.

85. Use of the therapeutic composition of any one of embodiments 71-74, for the treatment of a disease or disorder associated with reduced GCase activity.

86. Use of the therapeutic composition of any one of embodiments 71-74, for the treatment of Gaucher's disease.

87. Use of the therapeutic composition of any one of embodiments 71-74, for the treatment of a Lewy body disease (LBD).

88. The use of embodiment 87, wherein the LBD is Parkinson's disease, Parkinson's disease dementia, or dementia with Lewy bodies (DLB).

89. Use of the therapeutic composition of any one of embodiments 71-74, for the treatment of Parkinson's disease.

90. A recombinant adeno-associated viral (rAAV) nucleic acid vector for increasing expression of GBA1 in a cell, the vector comprising a promoter operably linked to a nucleic acid sequence encoding GBA1, wherein the cell exhibits (i) reduced activity of the β-Glucocerebrosidase (GCase) enzyme encoded by GBA1 and/or (ii) reduced expression of GBA1 prior to being introduced with the rAAV vector, optionally as compared to a reference cell from a subject without Parkinson's disease. 91. The rAAV vector of embodiment 90, wherein the cell comprises a variant of GBA1 associated with Parkinson's disease.

92. The rAAV vector of embodiment 91, wherein the variant of GBA1 comprises a single nucleotide polymorphism (SNP) that is associated with Parkinson's disease.

93. The rAAV vector of embodiment 92, wherein the SNP is rs76763715.

94. The rAAV vector of embodiment 93, wherein the rs76763715 is a cytosine variant.

95. The rAAV vector of any one of embodiments 90-94, wherein the variant of GBA1 comprising a SNP encodes a serine, rather than an asparagine, at amino acid position 370 (N370S).

96. The rAAV vector of embodiment 94 or embodiment 95, wherein the wild-type form of GBA1 comprises a thymine instead of the cytosine variant.

97. The rAAV vector of embodiment 92, wherein the SNP is rs421016.

98. The rAAV vector of embodiment 97, wherein the rs421016 is a guanine variant.

99. The rAAV vector of any one of embodiments 92, 97, and 98, wherein the variant of GBA1 comprising the SNP encodes a proline, rather than a leucine, at amino acid position 444 (L444P).

100. The rAAV vector of embodiment 98 or embodiment 99, wherein the wild-type form of GBA1 comprises an adenine instead of the guanine variant.

101. The rAAV vector of embodiment 92, wherein the SNP is rs2230288.

102. The rAAV vector of embodiment 101, wherein the rs2230288 is a thymine variant.

103. The rAAV vector of any one of embodiments 92, 101, and 102, wherein the variant of GBA1 comprising the SNP encodes a lysine, rather than a glutamic acid, at position 326 (E326K).

104. The rAAV vector of embodiment 102 or embodiment 103, wherein the wild-type form of GBA1 comprises a cytosine instead of the thymine variant.

105. The rAAV vector of any one of embodiments 90-104, wherein the cell is a dopaminergic neuron progenitor cell or a dopaminergic neuron.

106. The rAAV vector of any one of embodiments 90-105, wherein the cell, optionally the dopaminergic neuron progenitor cell, is derived from a pluripotent stem cell (PSC), optionally an induced pluripotent stem cell (iPSC).

107. The rAAV vector of any one of embodiments 90-106, wherein a plurality of the cell, optionally the dopaminergic neuron progenitor cell, were differentiated from pluripotent stem cells (PSCs), optionally induced pluripotent stem cells (iPSCs), by a method comprising:
(a) performing a first incubation comprising culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3B) signaling; and
(b) performing a second incubation comprising culturing cells of the spheroid in a substrate-coated culture vessel under conditions to neurally differentiate the cells.

108. The rAAV vector of embodiment 107, wherein the cells are exposed to the inhibitor of TGF-β/activin-Nodal signaling and the at least one activator of SHH signaling up to a day at or before day 7.

109. The rAAV vector of embodiment 107 or embodiment 108, wherein the cells are exposed to the inhibitor of BMP signaling up to a day at or before day 11.

110. The rAAV vector of any one of embodiments 107-109, wherein the cells are exposed to the inhibitor of GSK3β signaling up to a day at or before day 13.

111. The rAAV vector of any one of embodiments 107-110, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

### VIII. Examples

The following examples are included for illustrative purposes only and are not intended to limit the scope of the invention.

### Example 1: Differentiation of iPSCs into Dopaminergic Neuron Progenitors

A. Generation of iPSCs

Fibroblasts from a human donor (“Donor 1”) having idiopathic Parkinson Disease (PD) were obtained. The Donor 1 cell line was reprogrammed into induced pluripotent stem cells (iPSCs) using CytoTune™-iPS 2.0 Sendai Reprogramming Kit (ThermoFisher), and RNA sequencing information was used to confirm the pluripotency of the cells using PluriTest™.

Donor 1 iPSCs were subjected to an exemplary dopaminergic (DA) neuronal differentiation protocol. Briefly, iPSCs were maintained by plating in 6-well plates (e.g., laminin-coated plates) at 200,000 cells per cm². The cells ere cultured without feeder cells in mTeSR™ 1-based media until they reached approximately 90% confluence (day 0). The iPSCs were then washed with sterile PBS and detached from the 6-well plates by enzymatic dissociation with Accutase™. The collected iPSCs were then used in the subsequent differentiation protocol.

### B. Differentiation Protocol

The collected iPSCs were re-suspended in “basal induction media” (see below) and seeded under non-adherent conditions using 6-well or 24-well AggreWell™ plates. The cells were seeded under conditions to achieve the following concentrations: 500 cells/spheroid; 1,000 cells/spheroid, 2,000 cells/spheroid; 3,000 cells/spheroid; 10,000 cells/spheroid; or 15,000 cells/spheroid. Following seeding of the cells, the 6-well or 24-well plates were immediately centrifuged at 200×g or 100×g for 3 minutes, respectively. Beginning at day 0, the media was supplemented with various small molecules as described below. The cells were cultured for 7 days, with media replacement as detailed below, to form spheroids. On day 7, the resulting spheroids were dissociated into single cells by enzymatic dissociation with Accutase™. The cells were plated as monolayers at a concentration of 600,000 cells/cm² on substrate-coated 6-well plates (e.g., laminin-coated plates) for the remainder of culture, and were further supplemented with nutrients and small molecules as described below.

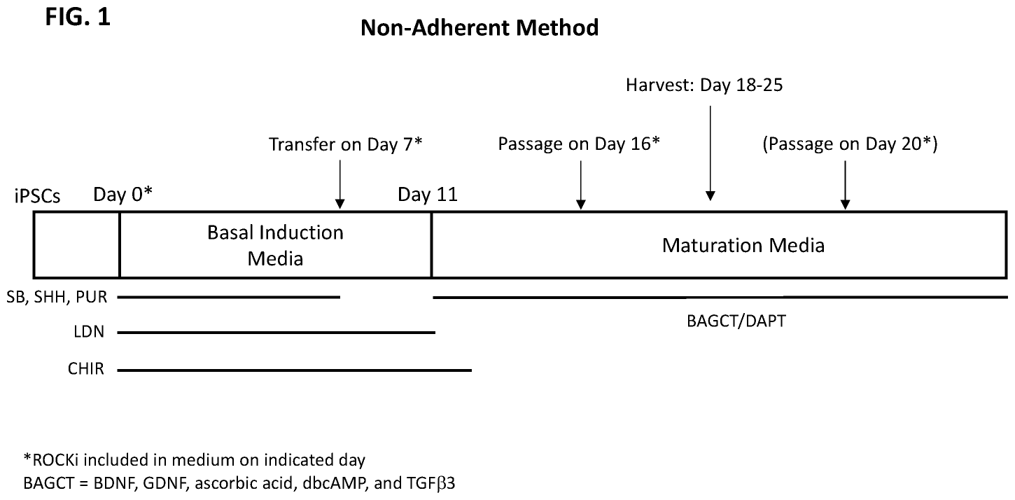

A schematic of the exemplary non-adherent differentiation protocol is shown in FIG. 1 and Table E1, which depict the small molecule compounds that were added at various days during the differentiation method. From days 0 to 10, cells were cultured in the basal induction media, which was formulated to contain Neurobasal™ media and DMEM/F12 media at a 1:1 ratio (and with N-2 and B27 supplements, non-essential amino acids (NEAA), GlutaMAX™, L-glutamine, β-mercaptoethanol, and insulin), and was supplemented with the appropriate small molecule compound(s). From days 11 to 25, cells were cultured in a “maturation media” (Neurobasal™ media containing N-2 and B27 supplements, non-essential amino acids (NEAA), and GlutaMAX™), and were supplemented with the appropriate small molecule compound(s). The basal induction media also included a serum replacement.

TABLE E1
Differentiation Protocol

| Day | Media | Small Molecules | | | | | | | |
|---|---|---|---|---|---|---|---|---|---|
| 0* | Basal | 5% S | LDN | SB | SHH | PUR | CHIR | ROCKi | |
| | Induction | | | | | | | | |
| 1 | Basal | 5% S | LDN | SB | SHH | PUR | CHIR | | |
| | Induction | | | | | | | | |
| 2 | Basal | 2% S | LDN | SB | SHH | PUR | CHIR | | |
| | Induction | | | | | | | | |
| 3 | Basal | 2% S | LDN | SB | SHH | PUR | CHIR | | |
| | Induction | | | | | | | | |
| 4 | Basal | 2% S | LDN | SB | SHH | PUR | CHIR | | |
| | Induction | | | | | | | | |
| 5 | Basal | 2% S | LDN | SB | SHH | PUR | CHIR | | |
| | Induction | | | | | | | | |
| 6 | Basal | 2% S | LDN | SB | SHH | PUR | CHIR | | |
| | Induction | | | | | | | | |
| 7* | Basal | 2% S | LDN | | | | CHIR | ROCKi | |
| | Induction | | | | | | | | |
| 8 | Basal | 2% S | LDN | | | | CHIR | | |
| | Induction | | | | | | | | |
| 9 | Basal | 2% S | LDN | | | | CHIR | | |
| | Induction | | | | | | | | |
| 10 | Basal | 2% S | LDN | | | | CHIR | | |
| | Induction | | | | | | | | |
| 11 | Maturation | | BDNF | GDNF | ascorbic | dbcAMP | CHIR | TGFβ3 | DAPT |
| 12 | Maturation | | BDNF | GDNF | ascorbic | dbcAMP | CHIR | TGFβ3 | DAPT |
| 13 | Maturation | | BDNF | GDNF | ascorbic | dbcAMP | | TGFβ3 | DAPT |
| 14 | Maturation | | BDNF | GDNF | ascorbic | dbcAMP | | TGFβ3 | DAPT |
| 15 | Maturation | | BDNF | GDNF | ascorbic | dbcAMP | | TGFβ3 | DAPT |

Day 16: 1^{st} Passage

| | | | | | | | |
|---|---|---|---|---|---|---|---|
| 16* | Maturation | BDNF | GDNF | ascorbic | dbcAMP | TGFβ3 | DAPT |
| 17 | Maturation | BDNF | GDNF | ascorbic | dbcAMP | TGFβ3 | DAPT |
| 18 | Maturation | BDNF | GDNF | ascorbic | dbcAMP | TGFβ3 | DAPT |
| 19 | Maturation | BDNF | GDNF | ascorbic | dbcAMP | TGFβ3 | DAPT |

Day 20: 2^{nd} Passage

| | | | | | | | |
|---|---|---|---|---|---|---|---|
| 20* | Maturation | BDNF | GDNF | ascorbic | dbcAMP | TGFβ3 | DAPT |
| 21 | Maturation | BDNF | GDNF | ascorbic | dbcAMP | TGFβ3 | DAPT |
| 22 | Maturation | BDNF | GDNF | ascorbic | dbcAMP | TGFβ3 | DAPT |
| 23 | Maturation | BDNF | GDNF | ascorbic | dbcAMP | TGFβ3 | DAPT |
| 24 | Maturation | BDNF | GDNF | ascorbic | dbcAMP | TGFβ3 | DAPT |
| 25 | Maturation | BDNF | GDNF | ascorbic | dbcAMP | TGFβ3 | DAPT |

S: Serum replacement;
LDN: LDN193189;
SB: SB431542;
SHH: recombinant mouse Sonic Hedgehog (rmSHH);
PUR: Purmorphamine;
CHIR: CHI99021;
ROCKi: Y-27632;
BDNF: recombinant human brain-derived neurotrophic factor (rhBDNF);
GDNF: recombinant human glial cell-derived neurotrophic factor (rhGDNF);
TGFβ3: recombinant human transforming growth factor beta 3 (rhTGFβ3);
dbcAMP: dibutyryl cyclic AMP;
Ascorbic: ascorbic acid;
*Indicates media supplemented with ROCK inhibitor (Y-27632)

Specifically, on day 0, the basal induction media was formulated to contain: 5% serum replacement, 0.1 μM LDN, 10 μM SB. 0.1 μg/mL SHH, 2 μM PUR, 2 μM of the GSK3β inhibitor CHIR99021, and 10 μM of the ROCK inhibitor Y-27632. The media was completely replaced on day 1 to provide the same concentration of the small molecule compounds as on day 0, except that no ROCK inhibitor was added. From days 2 to 6, the same concentration of the small molecule compounds as on day 1 was provided daily but by 50% media exchange; the concentrations of small molecules in the basal induction media were doubled on days 2 to 6, to ensure the same total concentration of compounds was added to the cell cultures. Also, the media on days 2 to 6 was formulated with 2% serum replacement.

On day 7 when the cells were transferred to substrate-coated plates, the basal induction media was formulated to contain: 2% serum replacement, 0.1 μM LDN, 10 μM SB. 2 μM CHIR99021, and 10 UM Y-27632. The media was replaced daily from days 8 to 10, with basal induction media formulated to contain 2% serum replacement. 0.1 μM LDN and 2 μM CHIR99021.

Starting on day 11, the media was switched to maturation media formulated to contain: 20 ng/ml BDNF, 0.2 mM ascorbic acid, 20 ng/ml GDNF, 0.5 mM dbcAMP, and 1 ng/ml TGFβ3 (collectively, “BAGCT”), 10 μM DAPT, and 2 μM CHIR99021. The media was replaced on day 12 with the same media formulation containing the same concentrations of small molecule compounds as on day 11. From day 13 until harvest, the media was replaced either every day (days 13-17) or every other day (after day 17) with maturation media formulated to contain BAGCT and DAPT (collectively, “BAGCT/DAPT”) at the same concentrations as on days 11 and 12.

On day 16, the cells were passaged by enzymatic dissociation with dispase and collagenase. Cells were re-suspended as small clumps and re-plated in maturation media that was further supplemented with the ROCK inhibitor.

On day 18, the differentiated cells were harvested by enzymatic dissociation, and analyzed by immunohistochemistry for markers of midbrain DA neurons, including FOXA2 and tyrosine hydroxylase (TH), or cryofrozen for downstream use. In some embodiments, the differentiated cells are harvested on day 20. If differentiation past day 20 is desired (such as until day 25), the cells are passaged on day 20 by enzymatic dissociation with dispase and collagenase, re-suspended as small clumps, and re-plated in maturation media supplemented with the ROCK inhibitor.

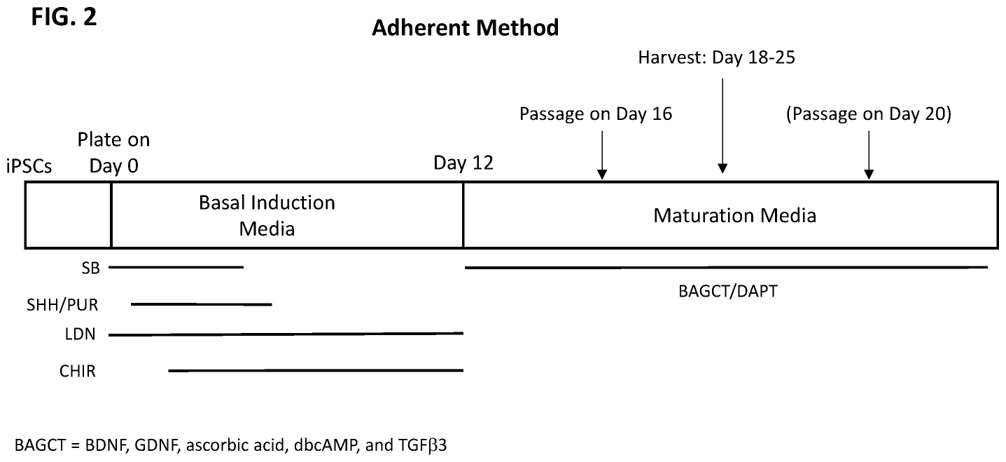

In some cases, differentiated cells were generated from iPSCs by an alternative method, in which the cells were initially plated in 6-well plates (e.g., laminin-coated plates) on day 0 and remained plated for the duration of the differentiation protocol (“adherent method”), such as until harvest at day 20. The adherent method also differed from the non-adherent method, in that the small molecules were added on different schedules (FIG. 2).

### Example 2: AAV-Based Modulation of GBA1

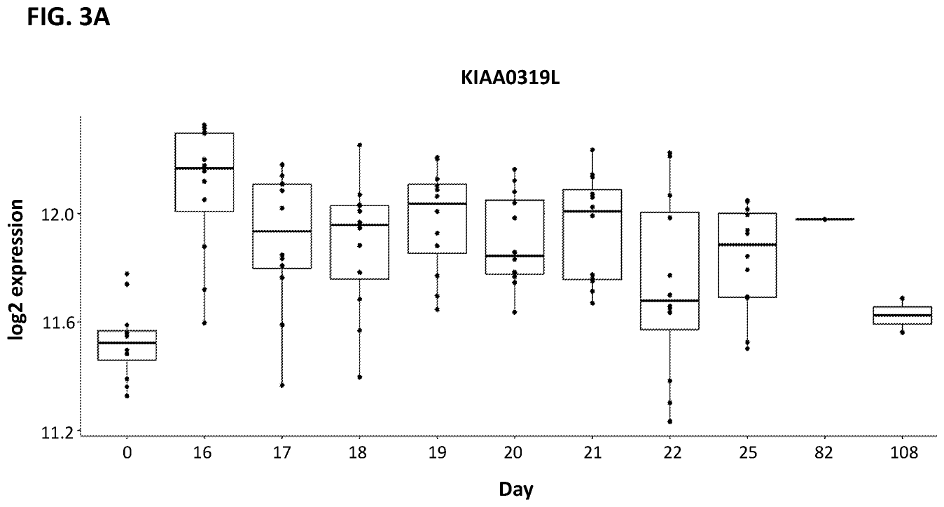

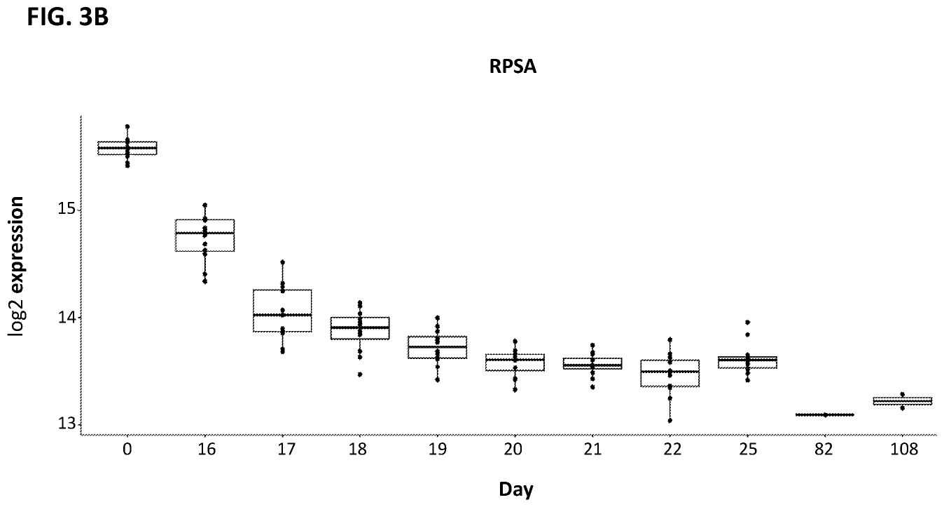

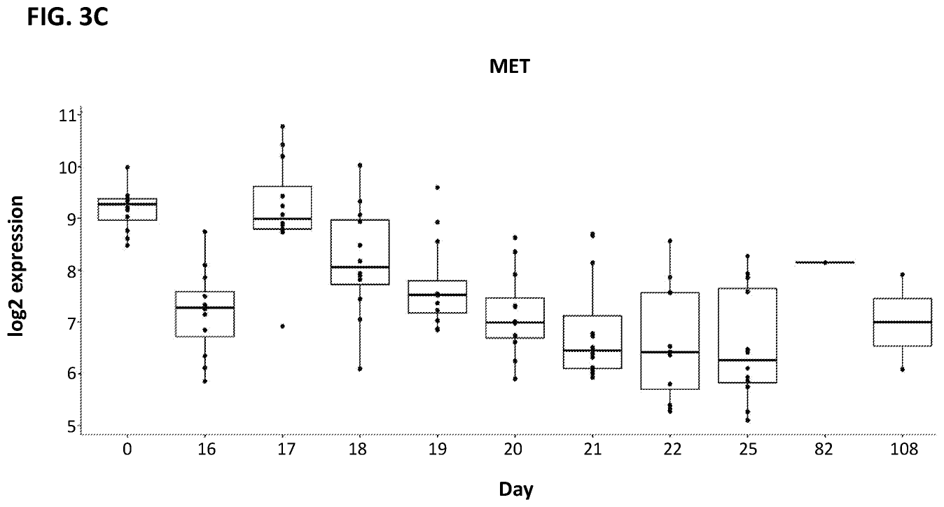

Adeno-associated virus (AAV) vectors were designed for the transduction of Donor 1 iPSCs differentiated by the exemplary adherent method described in Example 1. AAV vectors of either the AAV7m8, AAV9, or AAV-LK03 serotype were designed to contain a human GBA1 transgene under a ubiquitin C (UBC) promoter, which is known to be active in neurons. To determine an appropriate time to transduce the differentiated cells with the vectors, the expression levels of entry receptors for various AAV serotypes were assessed. In particular, expression of laminin receptor (LAMR/RPSA; encoded by RPSA) and AAV receptor (AAVR; encoded by KIAA0319L), entry receptors for AAV9, and hepatocyte growth factor receptor (HGFR; encoded by MET), an entry receptor for AAV-LK03, was analyzed in iPSCs (“day 0”) and differentiated cells (“day 16” through “day 108”). As shown in FIG. 3A, expression of KIAA0319L remained high in differentiated cells from days 16 to 25. Expression of RPSA was observed to be highest in differentiated cells at day 16 and was maintained through day 25 (FIG. 3B). Expression of MET was observed to be highest in differentiated cells at days 17 and 18, and was also maintained through day 25 (FIG. 3C).

Based on the findings that assessed genes encoding the AAV entry receptors tended to be expressed in differentiated cells from day 16 through day 25, Donor 1 iPSCs differentiated by the exemplary adherent method described in Example 1 and previously harvested and cryopreserved on day 18 were thawed and transduced the following day (day 19) with the AAV7m8, AAV9, or AAV-LK03 vector. Briefly, differentiated cells were seeded at 600,000 cells/cm² and transduced with between 12,500 to 125,000 viral particles per cell of the AAV9 vector, or between 3,125 and 75,000 viral particles per cell of the AAV7m8 or AAV-LK03 vector for 16 hours, followed by subsequent washing steps. Cells were cultured until day 25 or day 32, at which point the cells were harvested for analysis of transduction efficiency and Gcase activity. Non-transduced cells from Donor 1 served as a non-transduced control (NTC). Cells transduced with the AAV7m8 and AAV-LK03 serotype vectors could not be harvested on day 32 due to toxicity. In some embodiments, the cells are transduced on day 16, and remain in culture to continue differentiating until they are harvested on day 20.

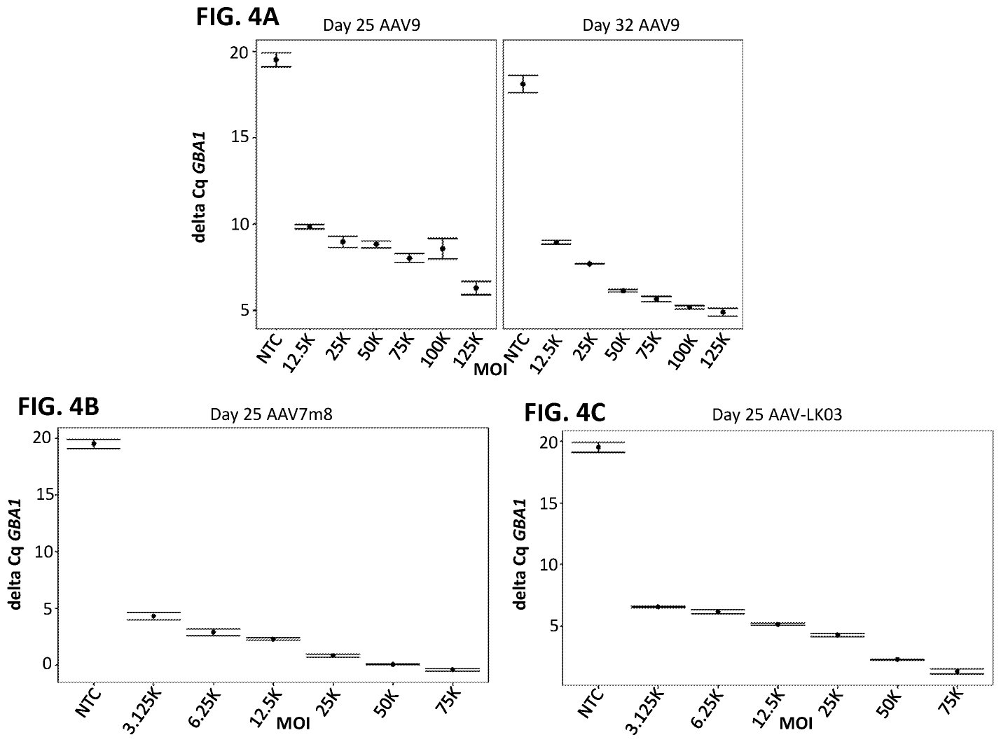

GBA1 expression was analyzed in cells from Donor 1 that were transduced with the three different AAV-UBC-hGBA vectors by qRT-PCR, normalizing GBA1 expression to GAPDH expression. All three serotype vectors were observed to yield a dose-dependent increase in expression of GBA1 in transduced cells, as compared to non-transduced cells from the same donor (FIGS. 4A-4C; a lower delta Cq value corresponds to higher expression of GBA1). GCase activity was also assessed in differentiated cells transduced on day 19 with the three different AAV-UBC-hGBA vectors, and compared to: non-transduced cells from another donor having idiopathic PD (ID-PD); non-transduced cells from a donor having a PD risk variant identified as SNP rs76763715 caused by the presence of a cytosine in place of a thymine (C>T), which causes an amino acid substitution of asparagine to serine at position 370 (N370S) in the beta-glucocerebrosidase (GCase) enzyme encoded by the beta-glucocerebrosidase (GBA1) gene (N370S); and non-transduced cells from Donor 1 (NTC). GCase activity was determined by an enzymatic activity reaction wherein protein isolated from cells was combined with the 4-methylumbelliferyl beta-D-glucopyranosidase (4-MBDG) substrate. Cleavage of the substrate by GCase yielded 4-methylumbelliferone (4-MU), the concentration of which was measured by comparing its fluorescent intensity to a standard curve.

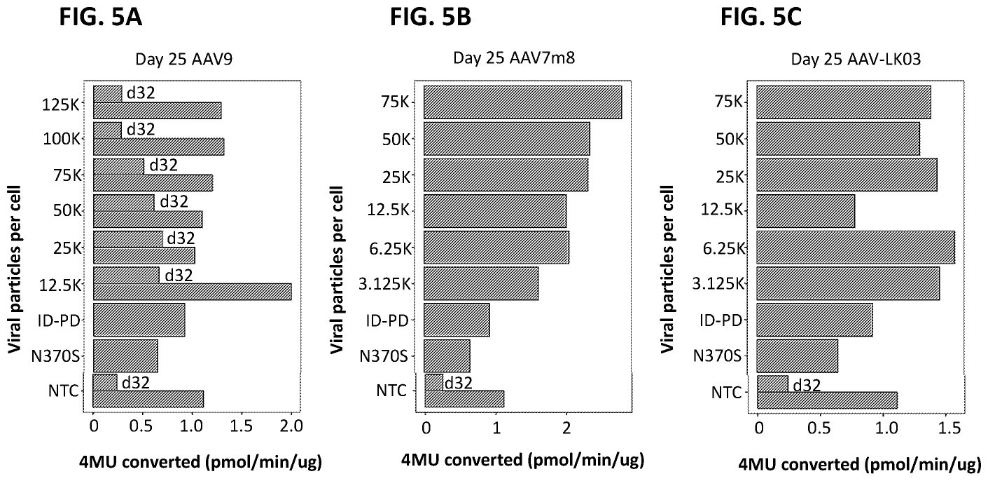

All three AAV vectors were observed to increase GCase activity in transduced cells from Donor 1, as compared to non-transduced cells from Donor 1 (NTC) (FIGS. 5A-5C; all cells harvested at day 25, except where harvest at day 32 is indicated by “d32”).

Expression levels of neuronal differentiation markers TH and FOXA2 were assessed in differentiated cells transduced with AAV9 and harvested on day 32. Transduction with the AAV9-UBC-hGBA vector was not observed to affect the fate of differentiated cells, as TH and FOXA2 expression was present in both transduced and non-transduced cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Sequences

| SEQ ID | | |
|---|---|---|
| NO | Sequence | Description |
| 1 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (wildtype) |
| | LHNFSLPEEDTKLKIPLIHRALQLAQRPVSLLASPWTSPTWLKINGAV | amino acid |
| | NGKGSLKGQPGDIYHQTWARYFVKFLDAYAEHKLQFWAVTAENEPS | sequence |
| | AGLLSGYPFQCLGFTPEHQRDFIARDLGPTLANSTHHNVRLLMLDDQ | |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 2 | ACAGAAGTACAATGCTGATTATGATCTGTCAGCTCAGCAAGGGGC | Human |
| | AGACACCCTGGCCTTCATGTCTCTCCTGGAGGAGAAGTTGATCCCG | GBA1 |
| | GTGCTGGTGAGTGTGCCCAGACCTCCCAGCATCCATGGCCAGCCG | (wildtype) |
| | GGGAGGGGACGGGAACACACAGACCCACACAGAGACTCAGGAGA | nucleotide |
| | GCATGGAGGTCAGAAGCCCACCTTGAATCAGACAGGTGCACTGGC | sequence |
| | TCAGACCTGCCTGTTTCTTCCTGCCCACCCAATCCAGGTACATACT | |
| | TTTTGGATAGACACCAAGAACTACGTAGAAGTGACCCGGAAGTGG | |
| | TATGCAGAGGCTATGCCCTTTCCCCTCAACTTCTTCCTGCCTGGCC | |
| | GCATGCAGCGGCAGTACATGGAACGGCTACAGCTGCTGACTGGGG | |
| | AGCACAGGCCTGAGGACGAGGAAGAGCTGGAGAAGGAGGTAGCT | |
| | CTGAGACCGGGGGCTATTGTATGAGATGAGCCCCAAGGATGCTGG | |
| | CCAGGAATGGGAGTGCTTAGGTGCGGAGGTGGCACTGTTCCCGCA | |
| | GCTGCAAGCCTACCTGTGTCGCCCCTACAGCTGTACCGAGAGGCTC | |
| | GGGAGTGTCTGACCCTGCTCTCTCAGCGCCTGGGCTCTCAAAAGTT | |
| | CTTCTTTGGAGATGCGTGAGTCTGACTCCAAGAGGGTAATGGGTG | |
| | GCTTGGAAGAAGATACAGGTTCAGATGGAGCAGCTGGAGCTGGGG | |
| | CTGGGGCTGGGGCTGGCTCAGGCTCTGGATAGGAGGTCCCTGAGA | |
| | CAGATACTGGCCCTGGTGACAGTGGGGCTGTGCGTGGGGCCAGAG | |
| | CCTTCTCAGAGGTACAAAAGGGTAGGGTGGGAGGGCAGCCAGGCA | |
| | CAGGAAGGGCCTGAAGAGCTGTGGGGCACTGAGTGTGCCCTTTAT | |
| | GCAGCCCTGGGATAGAGCCCTATTCAGGGCCAGGCTGGCGCCACC | |
| | TGGGGATCTCTCCCCATACCAGGTCTAGAACTGTGTGTCCTGTCCT | |
| | TCCCTGGTGGCCGCCTGCTGCCCAGAGCCCACCTCCCAAGGCTGAC | |
| | TCTTCCTCCAGCTCCATCTTTACCCCTTCTACCCCAGTGGTTCTCCT | |
| | CCATCCCACCCTTCTCTCTCTGCTCCAGCCCTGCCTCCTTGGACGCC | |
| | TTCGTCTTCAGCTACTTGGCCCTGCTGCTGCAGGCAAAGCTGCCCA | |
| | GTGGGAAGCTGCAGGTCCACCTGCGTGGGCTGCACAACCTCTGTG | |
| | CCTATTGTACCCACATTCTCAGTCTCTACTTCCCCTGGGATGGAGG | |
| | TAAGGGGCAGATGGGAGGGGCAGCCCTGGGGAGAGTGGGCAGGG | |
| | ATCCAAGAACTAGTTCTCCTAACACACCTTCCTTCCTTGACCCTCA | |
| | GCTGAGGTACCACCGCAACGCCAGACACCAGCAGGCCCAGAGACT | |
| | GAGGAGGAGCCATACCGGCGCCGGAACCAGATCCTATCTGTGCTG | |
| | GCAGGACTGGCAGCCATGGTGGGCTACGCCTTGCTCAGCGGCATT | |
| | GTCTCCATCCAGCGGGCAACGCCTGCTCGGGCCCCAGGCACCCGG | |
| | ACCCTGGGCATGGCTGAGGAGGATGAAGAGGAATGATTTGTCCTC | |
| | ACGCTCCCAAGACTGGTTTTTCTACTCTCATGCATTCCAGAGGCCC | |
| | CCGTGCCTCCTCGTTGTTGGTACAGCCGGACACGGGGTGCTGCCAC | |
| | CCAGAATAAAGCCACTCACACTGACTGGGCTCAAACATTTTCTCCT | |
| | TTAAGAGCTGCCATTTTTCCTGGCTGGTGCCATAGGAATCATCTGG | |
| | GTGCCTGGGCACACCCGCTGCTGCTTTAAGGCTTCCGCCCTGATGC | |
| | TGACACTGCTGCTCCACGGGCCCAGTTCTGCATCTCCAGGAAAGAC | |
| | AAACAGTCTCCAGTTTTGGGCCCAGCTTTCCTAGTCTCTTCTTTTCC | |
| | TTACCCTCAGCCCTGATCTTGTGTTTGTACGGACAGTGAGCTCACC | |
| | CTAGGCCTGGACCCAGGCCCAGTTTCCAAAGCAAGCAGCACACAG | |
| | CGCATGTTCACATAAGCATGGGGGCTGGGGGGACACTGGGGCTTA | |
| | CTGATCTTTTTCTAGGGGCCTCCAGCCCCTGGCACCACCTAGAGGG | |
| | GAAAGTGAGTCACCCAAACCATTGCCCCTGGGCTTACGTCGCTGTA | |
| | AGCTCACACTGGCCCTGCTGTGCCCTCTTTAGTCACAGACAGCGTG | |
| | TGAGCTGACTCTGTCCCTTTAATGCCCAGGCTGAGCCCAGTGCCTC | |
| | CTTGAGTATCTGCTCCATCACTGGCGACGCCACAGGTAGGTGTGAA | |
| | TGGAGTAGCCAGGTGAGATTGTCTCCAGGAAGCCCACAGCAGGAT | |
| | CCTTGATGGTAAGAGGCACATCCTTAGAGGAGCTAGGGAGCAGGG | |
| | AGGAGAAGCTGAGAGTGTGATCCTGCCAAGGCCCCCAACGCTGTC | |
| | TTCAGCCCACTTCCCAGACCTCACCATTGCCCTCACCGGTTTAGCA | |
| | CGACCACAACAGCAGAGCCATCGGGATGCATCAGTGCCACTGCGT | |
| | CCAGGTCGTTCTTCTGACTGGCAACCAGCCCCACTCTCTGGGAGCC | |
| | CTCAGGAATGAACTTGCTGAATGTGGGAACAGATGGTCAGAGTCC | |
| | CTCGGGGTACCTCCCATGAAACCCTCATCTAAGAAGTCACCCACCC | |
| | ACGGACCCACCCCATAACTCCTGCAGAGGCTCTGCCCTGGCTCTCT | |
| | AGGCCTGGAGCCATGCTGCTGGGCACTGACCCTGCTTTTCTGCATC | |
| | GCAGTCCAGCCTCAGGCATTGGGGTTTTCTGTTGCTACCTAGTCAC | |
| | TTCCTGCCTCCATGGTGCAAAAGGGGATGGGTGTGCCTCTTCCGAG | |
| | GTTCCACCCTGAACACCTTCCTGCTCCCTCGTGGTGTAGAGTGATG | |
| | TAAGCCATCCGATGTAGGAGATGATAGGCCTGGTATGGAATGGGG | |
| | GTGCCCGCCCTCCACTCACCTGAAGTGGCCAAGGTGGTAGAACAT | |
| | GGGCTGTTTGTAAAACGTGTCCTTGGTGATGTCTACAATGATGGGA | |
| | CTGTCGACAAAGTTACGCACCCAATTGGGTCCTCCTTCGGGGTTCA | |
| | GGGCAAGGTTCCAGTCGGTCCAGCCGACCACATGGTACAGGAGGT | |
| | TCTAGGGTAAGGACAAAGGCAAAGAGACAAAGGCGCAACACTGG | |
| | GGGTCCCCAGAGAGTGTAGGTAAGGGTCACATGTGGGAGAGGCAG | |
| | CTGTGGGTAGGTCAGCCCTGTGAGGGGCACATTCCTTAGTAGCTAA | |
| | GGAGTTGGGGGTGTGAAGATCCAGGCATCTCAAGGGGAGCTGAGA | |
| | AGTCTGAGGCAGCTGCAAGTGCCTCAGTAGTTGCAAAAGGGGCAA | |
| | TGAGGTGTGCAGACCTGTGAAGGAAAGGGAAGATAGGGAATCAT | |
| | GGTTCCCCAGAGTTGCTCAAAAGGGCAGGCTAGCTGGGGAAAGCT | |
| | GGACAGGAAGGGCTTCTGTCAGTCTTTGGTGAAACTAGTAAGAGG | |
| | TCTGAGGTCTGCTTTGCAGGAAGGGAGACTGGGGTGGCTTACCGT | |
| | GATGATGCTGTGGCTGTACTGCATCCCTCGATCCCAGGAGCCTAGC | |
| | CGCACACTCTGCTCCCAGAACTTGGAGCCCACACAGGCCTCTGAG | |
| | GCAAAGAGCATGGTGTTGGGGAACAGGCGGTGTGTCTCCCCTAGG | |
| | GTGGCTTTGGCTGGAGCCAGAAAGTCCAGGTACCAATGTACAGCA | |
| | ATGCCATGAACATATTTAGCTGCTTCTGGGTCTGTCAGTACCTGCA | |
| | AAGGAAGAGCAACTGATCCTGGACCTTGCACACAGGCTTCTGGAA | |
| | CTTCTAGTTCCTGTTGTAGGAATCCTGGAGTTGGGTGACGGGAAGA | |
| | ATGCAACTAGAGAGGTTTGGGGAGATTTTTTTTTGTTTTTGAGACA | |
| | GGATGTCACTCTGTCACTCGGGCTGGAGTACAGTGGCGCAATCAC | |
| | GACTCACTGCAGCCTTGACTTCCTAGGGTCAACTGATCCTCCCACA | |
| | TTAGCCTCCTGAGTAGCTGGGACTACACGGGTGCCACACCCAGCT | |
| | AATTTGTGTGTGTATGTGTGTGTATGTATGTGTGTGTGTGTATATAT | |
| | ATATACATATAAACACATATATATGTATATACACATACATACATGA | |
| | ACCACCACCCCCAGCCTAGATAGTTTTGTTTTGTTTTGTTTCGAGAT | |
| | GGAGTCTCGCTCAGCCTCCCAGGCTGGAGTGCAGTGGCGCGATCT | |
| | CGGCTCACCGCAACCACCATCTCCCGGGTTCAACTGATTCTCCCTT | |
| | CTCAGCCTCCTGAGTAGCTGGGATTACAGACACCCACCATCATGCC | |
| | CAGATAATTTTTTTTTTTTGTATTTTAGTAGACACAGGGTTTCAACA | |
| | CGTTGGCCAGGGTGGTCTTAAACTCCTGACCTCAGGTGATCACCCG | |
| | CCTCGGCTTCCCAAAGTGCTGGGTTTGCATGAGTGAGCCACCTCGC | |
| | CCAGCCCCTAGAAAGGTTTCAAGCGACAACTGTGGGATCCATGGC | |
| | ACCCTGGAGGTCCAGGGGAATGGTGCTCTAGGAATCCATAGTTGG | |
| | GTAGAGAAATCGCTCTAAGTTTGGGAGCCAGTCATTTGGATGCTG | |
| | GATTTGAAGGTCACTGGAGCACCATGGAGGTCCAGGCCTTACCAC | |
| | CTTTGCCCAGTGGGGCAGCAGCAAGCGTTGGTCATCCAGCATGAG | |
| | TAGGCGGACATTGTGGTGAGTACTGTTGGCGAGGGTAGGACCTAG | |
| | GTCACGGGCAATGAAGTCTCGCTGATGTTCAGGGGTGAAGCCCAG | |
| | GCACTGGAAGGGGTATCCACTCAACAGCCCAGCAGAAGGCTCATT | |
| | TTCAGCTGTCACTGCCCAGAACTGTAACTTGTGCTCAGCATAGGCA | |
| | TCCAGGAACCTGGCAAGAGAAAGGTCATGAATGATCCGGCCAAGA | |
| | AAGTGGACCAGACCAGCTGGGTGTGGTGGCTCACACCTGTAATCC | |
| | CAGCACTTTGGGAAGCCGAGGCAGGTGGATCACTTGAGTTCAGGA | |
| | GTTCGAGAACAGCCTGGCGAAACCCCGTCTCTACTAAAAATAGAA | |
| | AAATCAGCTGGGCCTGGTGGCAGGCGCCTATAATCCCAGCTACTT | |
| | GGAAGGCTGAGGCAGGAGAATTGCTTGAACTCAGGAGGCAGAGG | |
| | TTACAGTGAGTGAAGATGGCGCCACAGCACTCCAGCCTGGGTGAC | |
| | AGAGAGAGAGACTCCTTCTCAAAAAAAAAAAAAAGAAGAAAAAT | |
| | AAAAAGAAAGTGGGCCAGACCGAGAGAACAGGAAGCCTGATGGA | |
| | GTGGGCAAGATTGACAGGCCCAAGGCTGAAAGGCCCAGAAGGTA | |
| | GAAAGGTGAGCTGAGGACAGGCAGATCTGGAAGTGGAACTAGGTT | |
| | GAGGGTTGGGACACAGATCAGCATGGCTAAATGGGAGGCCAGTCC | |
| | TGATCCCACATCCTTGCTGATCCCTTACTTCACAAAGTATCTGGCC | |
| | CAGGTCTGGTGGTAGATGTCTCCGGGCTGTCCCTTGAGTGACCCCT | |
| | TCCCATTCACCGCTCCATTGGTCTTGAGCCAAGTGGGTGATGTCCA | |
| | GGGGCTGGCAAGGAGTGAAACGGGACGCTGGGCCAACTGCAGGG | |
| | CTCGGTGAATCAGGGGTATCTAGAGACAAAGGTAGTGAAGAGAGA | |
| | AGCACCCAGAGTTGGAACACATACTAGCCCAACCAGTGCATCCGG | |
| | TTCAGCCATTAGCCTCCACCCTCCCACCCCCAGGACAAAACAGCA | |
| | GGGGACAAAATGTCTGTACAAGCAGACCTACCCTACAGTTTCTCA | |
| | ACCCCCAGACATCAGGGCCCTCAGGGCCTGAAAAAGCTAGAATGC | |
| | CTACCTTGAGCTTGGTATCTTCCTCTGGGAGGCTGAAGTTGTGCAA | |
| | CTGGAAATCATCAGGGGTGTCTGCATAGGTGTAGGTGCGGATGGA | |
| | GAAGTCACAGCTGGCCATGGGTACCCGGATGATGTTATATCCGATT | |
| | CCTACAGAAAAGGATGATCAAGATATGGTAGTCCGAGTCAATAGG | |
| | AGAGTATGGGACTCTGCTTATCACTTGCCAGTCCTAATAGTGTCTG | |
| | AGTCAGGGCCAAAGGGAACTTGGGCTCCTGGGTTGGAACCTGTGG | |
| | AGGCTGGCACCTGGGTGAAGCGCAGGCCTTTCTGAGCCTGAGTCC | |
| | GTAGCAGTTAGCAGATGATAGGCGGTGAAATCTTATTTCACAGGG | |
| | CATTAAAACAGGAACCAAATGTCAGGGATGGGCAGAAGTCAGGGT | |
| | CCAAAGAAAGGGCAAAGAAAAGTGTCAGTGGCTCACGCATGTAAT | |
| | CCCAGCACTTTGGGAGGCCGATGTGGGCAGATCACGAGGTCAGGA | |
| | GTTCGCAATCATCCTGGCCAACATAGTGAAACCCTGTCTCTACTAA | |
| | AAATACAAAAAATTAGCCGGGCGTGGTGGCAGGCACCTGTAATCC | |
| | CAGGTACTCGAGAGGCTGAGACAGGAGAATCACCTGAACCCGGGA | |
| | GGTGGAGGTTGCAGTGAGCTGAGATTGTGCCACTGCACTCCAGCC | |
| | TGGGTGACAGTGCGAGACTCTGTCTCAAAAAAAAAAAAAGAAAA | |
| | AAGAAAAGTGTCTGCTGGGCTCGGTGGCTCACACTTGTAATTCCAG | |
| | CACTTCGGGAGGCCAAGGCAGGTATATCATTTGAGGTCAGGAGTT | |
| | TAAGACTAGCCTGGTCAACATGATGAAACCCTGTCTCTACTAAAA | |
| | ATACAAAAATTAGCCAGGTGGTAGTGGCGCACGCCTATAATTCCA | |
| | GCTACTCAGGAGGCTGAGGCAGGAGAATCACTATAGCCTGGGAGG | |
| | CAGAGGTTGCTGTGAGGGGAGATCACACCACTGCACTCCTGTCTC | |
| | GCCGACAGAATGGGCAGAGTGAGATTCTGCCTCAAAAAAATTTTA | |
| | AAAAAAGAAAAGAAAAACGAAAAGTTTCAATGGCTCTATGTCATC | |
| | TTGTCCCCTTCCTCCTCACCTTCTTCAGAGAAGTACGATTTAAGTA | |
| | GCAAATTTTGGGCAGGGGGTGACAGGGCAAGGATGTTGAGAGCAG | |
| | CAGCATCTGTCATGGCCCCTCCAAATCCCTTCACTTTCTGGAACTT | |
| | CTGTTCTGGCTGCAGGGTCAGTAGCAGGCCTGAGGACATCCACAG | |
| | GGAATAAGGGTATCAGTACCCAGCGGGAAACTCCATGGTGATCAC | |
| | TGACACCATTTACCTCTAGGAGGACCCAGCCTGGCCCAGGGGGTG | |
| | AGGGGTGTAATGGTTACCTGTGCCCGTGTGATTAGCCTGGATGGGC | |
| | CCCATACTCAGCTCCATCCGTCGCCCACTGCGTGTACTCTCATAGC | |
| | GGCTGAAGGTACCAAGGGCAGGAAAGGTCGGGGGGTCAAAGGAG | |
| | TCACAGTATGTGGCATTGCAGACACACACCACCGAGCTGTAGCCG | |
| | AAGCTTTTAGGGATGCAGGGGCGGGCACCTGGGAGGGAGGGAGT | |
| | ACAAGCAGAGTGAGGTCTGATGAAGACATGGAGAATGGACACATC | |
| | TGCTAGGAGAGACTGAACACGGTTTCAAAATTCCTCACCCCTTGGC | |
| | CGGGCGCAGGGGCTCACACCTGTAATCCTAGCACTTTAGGAGGCC | |
| | GAGGTGGGCGGATCACCTGAGGTGAGGAGTTTGAGACCTGCCTTG | |
| | CCAGCATGGTGAAACCCCGTCTCTACTAAAAATACAAAAATTAGC | |
| | TGGGCGTAGTGGTGGCCGCCTGTAATCCCAGCTACCTGGGAAGCT | |
| | GAAGCAAGAGAATCGCTTGAATCTGGGAGGCAGAGGTTGGAATGA | |
| | GCCAAAATTGCACCACTGCACTCCAGCCCAGGCAACAGAGTAAGA | |
| | CTCTGTTTCAAAAGAAAAAAAACAAAATCCTCACCCCAAAGTTGG | |
| | TCTCAGTCACTCAAAAGGATTTATTGAGCACCTACTAAAAGTCTAC | |
| | CACCAGCTTACTGGAAGGCTACCAAAGGACTATGAGGCAGAAGGG | |
| | AGGCTCTGTGCTACCTCCCCACTGCCTTGACTCACTCACCTGATGC | |
| | CCACGACACTGCCTGAAGTAGAAGCAATCCTGTGAGGCTGCCAGC | |
| | CATGATGCTTACCCTACTCAAAGGCTTGGGACATTCCTGAGGACAG | |
| | AATGAGGAATGACTGAAAAGCAAGCCCCTCTCCACCCCTCAACTA | |
| | CTCTCCTGGGCAGGGCTTAGCTGCCTTTGGGTGCCCATGGCCCGGT | |
| | CTCCCACATTCATTAGGACAAGGCCCACAGAAACCTGGGTGCAGC | |
| | TCTCCCTGCAACCCTTCTGATGACAACTCTCTGCCCACCCCAAATC | |
| | AGGATGCTCCCCACCACTCTTCCCACCCATTTCAACTTCGACCCCT | |
| | CCCTCCATCTGTGCCTTGCTCAAAGAGCCATGATGGCCCTGGATTC | |
| | AAAGAGAGTCTGTCATTCATTAAATTCCAGTGCCAGGATTCCAGA | |
| | AGCACTGTGACAATGCTGATTGGGAGCTCTCTCTCTTACCTCTCTG | |
| | GAAGGACTTGAAAACTCCATCCCCTCAGGGTCATTAGATGAAGAG | |
| | AAGACCACAGGGGTTCCAGAGTCTCTGAAGGATAGAGGATCCACT | |
| | AAACAAAAACAAGGATGCAGGTACCTGCCTTAGCTATAGGCACTA | |
| | GGTTAGCCCTGCAAGTAATTCCGGCTTCCCGATGTGGATGGGTCAT | |
| | GTGATGACTAGGAGCGTCACATGACACAGGAAGTGAGGCAATCAC | |
| | AGCCATATTTCTAAAGGGCAATTGGCTTCCTCTCATCTGTTACAGA | |
| | TTATATGCCCTATAAAACTCTGGAGGGCATGTATGGGTGACAACTT | |
| | TAGGAAGAGCCTAGAACACAGATTTGCATGGAGAATGGGGTAGAG | |
| | GCTCCTAAATCCCAGAGGATGGGAACCACAGCAGGCACACTTCTT | |
| | TTTTGATAGAAATGTCAAAAAGGTACAAATAAAGCTGATATAATT | |
| | TAAAAAAAAAAACTTCACTGTGCAAAATACTGACACTACCAAGAT | |
| | GTTTAAGACAGTCTTTGCCCTATAGAGGTGTGTGAGGCATGGAAGT | |
| | CAGACACACAGATATTTACAGATAAAGACAGAAACGGTAACAGGT | |
| | GTGCATGGAGAGCTCTCTGAGATGAGGAGGGACCATTCGTGGGTG | |
| | GGGAATTATCCAGGATGGCTTCACAGAGGAAAAAGTGAGGGGGGT | |
| | TATTAGCCAGTGAAGTGCAGGGCACAAGAGGGTGGGACACTGGCA | |
| | GTGGGATGACAGGACTGGAGGGAGGGAGTGGTCAGTGCGGCTCCT | |
| | CTGCAGCGTCCCTTGTTTCATCATCAGATGCAGATGGTAATAACTG | |
| | TTCTTCCTTCCTCACAAGACAGGGAGGTTTGTAAAGTCGTTCAAAA | |
| | ACCAAAGTGTTGTACAAAGCCATATCCTCAGTGGACACAAGGAGG | |
| | AAGCTGTCCATGGTGTGGCCTCATGAACCACATCAAATGAGATTTA | |
| | GCGGGAGTGGCACACACAGTCATGACCTGACTAATCCCAGCTCTC | |
| | AGCCCATTTCCTTGCCTGGAAAATGGAGGCAATGCCACAACCTCA | |
| | AAGGGTGGTTACTGCAGTCAGTGAGGTAAGTGCAGTGCCTGACCA | |
| | CTTGGTAGGCACCTGGGAACTACTTGTCTCTTGTTTGTATTTTTTGT | |
| | TGTTGTTGTTTTTTGAGACAGAGTCTCACTCTGTTGCCCAGGCTGG | |
| | AGTGCAGTGGCGTGATCTTGGCTCACTGCAACCTCCACCTCCTGGG | |
| | TTCGAGTGATTCTCCTGCCTCAGCCTCCCAAGTAGCTGGGATTACA | |
| | GGTGCCCTCCAACATGCTCAGCTAATTTTTTTATTTTTAGTAGAGAT | |
| | GGGGTTTCACTATGTTGGCCAGGCTGGTCTGAAACTCTGGACCTCA | |
| | GGTGATCTGCTGCCTTGGTATCCGAAAGTGCTGGGATTACAGGCAT | |
| | GAGCCACTGCGCTGGACCCCAGCCATCCTTTTTGTTCTTTAAATGA | |
| | TCTCAGTGAAGTCTTTCTAGATACACTCCAAGCAAAATTGATCATT | |
| | TCTTCTTCTAGGTTCCTCCAGTAATTTTTTTTTTTTTGGTTTTGAGAC | |
| | AGAGTCTTGCCCTGTTGCCCAGGCTGGAGTGCAGTGTGATCTCGGC | |
| | TCAAGCAATTGCCCTGCCTCAGCCTCCTGAGTAGCTGGGATTACAG | |
| | GAGCCCACCACCATGCCCAGCTAATTTTTGTATTTTTAGTAGAGAT | |
| | GGGGTTTCACCATGTTGGCCAGGCTGGTCTCGAACCCCTGACCTCA | |
| | GGTGATCCACCAGCCTCAGCCTCCCAAGGTGTTGGGATTATAGCA | |
| | ATGAGCCACCACACCCGGTCCCTCCAGTAATTAAGTACAGTCTCAG | |
| | TGAGACAGCAAGTTTGGAATCCTGGCTACACCATTTACTGGCTGTT | |
| | TGACTTTCAATAAATCAATCACTCTAAGCCTCTGTTTCATCTATAA | |
| | AAGGGGAGTGATAACTCCTACCTCACAGAATTGTTGTGAGGTTTG | |
| | AGTGTGATAATGTGTCTCTAGTACACTGCTTGACACTAAACATTGC | |
| | AACATGTCAGGCCTCTGTTCCTGGAGTTCCTTGAAGGAATGTCTTA | |
| | TGCATTCTAAGTATCCTCGTAGATAGCCTGGCACAGGGGTAGCTGT | |
| | CAAGTTGTAGAATTGAACTAGTTGCTTCACTATGTCAGTAGCCACC | |
| | CCTTCCAGACTTCTCACTTTTCAAGGAAACATTGCACAGGATTTGT | |
| | TCTGGGTAGGGCAGGTAATATCTAGTACCTTACTTCCCTCAAGTTC | |
| | ATTCATCTCACAGATATTTCCTGAGCACATTCTACATTTGCCTCTCC | |
| | TGCTCTATTGAGTTTAGAAGTCCAAACATCTCCTTCCACTTCCCCTC | |
| | TGCATAGTGAGCCTCTTCTTTTTTGGCCAGGTACTGAGCAATTTTTT | |
| | GTTTGTTTGTTTGAGACAGGGTCTCCGTATGTTGCCCTGGATGGAG | |
| | TGTAGTAGCTTGATTTCGGCTCAGTGCAACCTCTGCCTCCAGAATA | |
| | AGCAATCCTCCCACCTCAGCCTCCAGAATAGCTGGGACTATAGGC | |
| | GCACACCACCACACCTGGCTAATTTCTGTATTTTTTGTAGAGACGG | |
| | GGTCTTACTGTGTTGCGCAGGTTGGTCTCAAACTCCTAAACTCAAA | |
| | GGATCCTCCCGCCTTGGCTTTGCAAAGTGCTGGGATTACAGGGTGA | |
| | GGCACTGCGCCCAGTCCGCTTGTTTTTTGTTTTGTTTTTTTTTTTGA | |
| | GACGGAGTATCTCTGTCACCCAGGCTGGAGTGCAGTGGCACGATC | |
| | TCGGTTCACTGCAACCTCCGCCTCCTAGGTTCAAGCGGAGGGTAGG | |
| | GACCAGTCCATCCTGGCACCCATCTGCAGCTCCAGTGCGAATCCCA | |
| | ACCCCGACGCTCGTCGCCGGGCTCCGTGAATGTTTGTCACATGTCT | |
| | GAAGAACGTATGAATTACATAACCTTCTTCCCACTCCACCCCTCAA | |
| | AAAGCAAGTGGATATAAAGACTTGAAGATTTTATAATCTCTTCTTC | |
| | ATTAGTAAAATCTGACCATCCTTACCGTTAAAAATAATAATGATGG | |
| | TTGGCCGGGCGCGGTGGCTCACGCCTATAATCACAGCTGTTTGGCA | |
| | GGCCGAGGCGGGCGGATCACGAGGTCAGGAATTGGAGACCAGCCT | |
| | GACCAACATGGTGAAACCCCGTCTCTACTAAAAGATACAAAAAAT | |
| | TAGCCGTGCGTGGTGGCGGGTGCCTGTAATCCCACGTACTTGGGA | |
| | GGCTGAGGTCGGAGAATCGCCTGAACCCGGGAGGCGGAGGTTGCA | |
| | GTGAGCCAAGAAGGCGCCATTACACTCCAGCCTGGGCGACAGGGC | |
| | GAGACTCCCTCAAAAAATAATAATAATAACAATAATAATAATAAT | |
| | GGTGAAAAAGGTTAAGTGCGAACGCAGGGAGGGGACAGCTAAGA | |
| | TCCAAAGGTCGAAATATTCATTACGCCTACCGTCGGCGAAGAGAA | |
| | ACAGCAGCCCCAACCGGAGGCAAAACGAAATCCCACCGCAGCCTG | |
| | CAAAGGCGCCTGGGCGGGACTGGAGACTGGGGCCCCGCGCAGTAA | |
| | GACTCTGAAGGCAGGATGCAGCCCGACCACCCGCAGCCGCGAGAA | |
| | AAGCAGCCCTGGGGAGTCGGGGGGGGACCTGGATTGGAAAAGAG | |
| | ACGGTCACTCATGCAGAGGCGGGACTCAGAGCCCTTCCTCAAGTC | |
| | TCATTGGTCAAGTTGAACGAACAAGTGTCGCTGGCGAGCCGGAGA | |
| | GAGAGAGAGAGCGAGAGCGAGAGAGCGAGCGAGAGAGAGCGAG | |
| | AGAGCGAGAGAGCGAGCGAGAGAGAGAGAGAGAGAGAGAGGAG | |
| | CCGGCGCGAGAACTACGCATGCGTGTCGGCGTTTTCCCGCCAGCA | |
| | CACTGTTGGTGGATGGGGGCGATTGAATTCCCACAGTGAGTCCAG | |
| | CCCACCGAAGCTCAGAGGATTCCTAACCTTCCTCTTTAGAGAGCCT | |
| | CAGGTTAGGGAACGTCCAGTGCGCAGAAGCTGCCCCGTGGGAATC | |
| | CCATTGTCCATCGCCTCCTAATGTTTCGTCCTGGCCGTGCCCTATCC | |
| | CTTCCTGAGGCTGGGTTGTTATGATGCTGAATTATTCAAGAAGTCT | |
| | TGCAGCCTGACGCCATCTCTGGGCAGTGCTCCTCCCACCTCCCTGT | |
| | CTTCCTTGGAGGGCACCACGTTGCCCCTACAAGCACAGGGTCCTGA | |
| | AGCTGTTTACAGGGCCCCACCCTGCCACTTTAGTATCTTAACGAAT | |
| | GTTTGTTGAGGACCTACTGAGTGTTAGACCCACTGCAATGAGCAA | |
| | GTTTCTGATCTTGGGACCTTAACATTCTATGGGACGACACAGATAT | |
| | ATAAACAAAAATAATAATTTTAGATAGTGATACATGCTGAAACCA | |
| | TGGGTATGTGGCCAGGCGCGGTGAGTCACACCTGTAATCCCAGCA | |
| | CTTTGGGAGACCGAGGCAGGCGGATTACTTGAGGTCAGGAGTTTG | |
| | AGACCCGCCTGGCCAACATGGTGAAACCCTATCTCTTCTAAAAATA | |
| | CAAAATTTAGCTGGGCGGGCATGGGGACGGGCGCCTGTAATCCCA | |
| | GCTACTCGGGAGGCTGAGGCAGGAGAATTGCTTGAACCCAGGAGG | |
| | TGGAAGGTTGCAGTGAGCCCAAATCACACCACTGCACTCCAGCCT | |
| | GGGTAACAAAGCAAGACCCTGTCTCAAAAAAAGACAAAATGAAA | |
| | CAAACAAACAAAAAAAAACATGGCTATGTGATAGAATGACTGCGA | |
| | TGGGGATTGGGTGCTCACTGAAATGGTGAGAATGGAGCTAAAACC | |
| | TGATATTAGGTGATAGCACCAGATATAGGGGCCAAGGGTTTCATG | |
| | CTTGGAAAACAGCAGGTGCAAACGTCCTTGTGCTGTGAGGAAATT | |
| | TGGCTGGAACAGAAGGTGGGCCAGATTGTAGCTGACCATAGTCTC | |
| | TGGGAGTTTGGAGATGATTCTAAATACAATGGGAAGCCATTAAAT | |
| | TGAGGCAGAGCCTTGATGTGACGTGCTTTATCATCATTTTGCTGAG | |
| | TGGAGAATGGATTGCTGGGAAAAGTGAAAGACTGATTCAGAGGTT | |
| | GTGGAGAGAGGATGGCTGGAGCTAGGTAGCTGGGGAGGTGGTGG | |
| | GATGTGGATGAATTCAGGATAGGATTGGTTGTGTTGGATATGGGA | |
| | AAAGAAGTCACCATGGTTCCTGAGCTTTTGGAATGTGCAAATGGG | |
| | TAATGGAGGTGCCATTGAGATGGGAACAGTGGAAAAATAACCGGT | |
| | GCTGGGGTGGGAATCAAGAGTTCAGTTGTGGCCATGTTGAATCTG | |
| | AAATGCCCATTAGACATCCAAGCAGACATGCTGAGTTGGATGGCG | |
| | CTCAT | |
| 3 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (N370S |
| | LHNFSLPEEDTKLKIPLIHRALQLAQRPVSLLASPWTSPTWLKINGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITSLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 4 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (L444P |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDPDAVALMHPDGSAVVVVLNRSSKDVPLT | |
| | IKDPAVGFLETISPGYSIHTYLWRRQ | |
| 5 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (E326K |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGKTHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 6 | ATGGAGTTTTCAAGTCCTTCCAGAGAGGAATGTCCCAAGCCTTTGA | Human |
| | GTAGGGTAAGCATCATGGCTGGCAGCCTCACAGGATTGCTTCTACT | GBA1 |
| | TCAGGCAGTGTCGTGGGCATCAGGTGCCCGCCCCTGCATCCCTAAA | codon- |
| | AGCTTCGGCTACAGCTCGGTGGTGTGTGTCTGCAATGCCACATACT | optimized |
| | GTGACTCCTTTGACCCCCCGACCTTTCCTGCCCTTGGTACCTTCAGC | nucleotide |
| | CGCTATGAGAGTACACGCAGTGGGCGACGGATGGAGCTGAGTATG | sequence |
| | GGGCCCATCCAGGCTAATCACACGGGCACAGGCCTGCTACTGACC | |
| | CTGCAGCCAGAACAGAAGTTCCAGAAAGTGAAGGGATTTGGAGGG | |
| | GCCATGACAGATGCTGCTGCTCTCAACATCCTTGCCCTGTCACCCC | |
| | CTGCCCAAAATTTGCTACTTAAATCGTACTTCTCTGAAGAAGGAAT | |
| | CGGATATAACATCATCCGGGTACCCATGGCCAGCTGTGACTTCTCC | |
| | ATCCGCACCTACACCTATGCAGACACCCCTGATGATTTCCAGTTGC | |
| | ACAACTTCAGCCTCCCAGAGGAAGATACCAAGCTCAAGATACCCC | |
| | TGATTCACCGAGCCCTGCAGTTGGCCCAGCGTCCCGTTTCACTCCT | |
| | TGCCAGCCCCTGGACATCACCCACTTGGCTCAAGACCAATGGAGC | |
| | GGTGAATGGGAAGGGGTCACTCAAGGGACAGCCCGGAGACATCTA | |
| | CCACCAGACCTGGGCCAGATACTTTGTGAAGTTCCTGGATGCCTAT | |
| | GCTGAGCACAAGTTACAGTTCTGGGCAGTGACAGCTGAAAATGAG | |
| | CCTTCTGCTGGGCTGTTGAGTGGATACCCCTTCCAGTGCCTGGGCT | |
| | TCACCCCTGAACATCAGCGAGACTTCATTGCCCGTGACCTAGGTCC | |
| | TACCCTCGCCAACAGTACTCACCACAATGTCCGCCTACTCATGCTG | |
| | GATGACCAACGCTTGCTGCTGCCCCACTGGGCAAAGGTGGTACTG | |
| | ACAGACCCAGAAGCAGCTAAATATGTTCATGGCATTGCTGTACATT | |
| | GGTACCTGGACTTTCTGGCTCCAGCCAAAGCCACCCTAGGGGAGA | |
| | CACACCGCCTGTTCCCCAACACCATGCTCTTTGCCTCAGAGGCCTG | |
| | TGTGGGCTCCAAGTTCTGGGAGCAGAGTGTGCGGCTAGGCTCCTG | |
| | GGATCGAGGGATGCAGTACAGCCACAGCATCATCACGAACCTCCT | |
| | GTACCATGTGGTCGGCTGGACCGACTGGAACCTTGCCCTGAACCCC | |
| | GAAGGAGGACCCAATTGGGTGCGTAACTTTGTCGACAGTCCCATC | |
| | ATTGTAGACATCACCAAGGACACGTTTTACAAACAGCCCATGTTCT | |
| | ACCACCTTGGCCACTTCAGCAAGTTCATTCCTGAGGGCTCCCAGAG | |
| | AGTGGGGCTGGTTGCCAGTCAGAAGAACGACCTGGACGCAGTGGC | |
| | ACTGATGCATCCCGATGGCTCTGCTGTTGTGGTCGTGCTAAACCGC | |
| | TCCTCTAAGGATGTGCCTCTTACCATCAAGGATCCTGCTGTGGGCT | |
| | TCCTGGAGACAATCTCACCTGGCTACTCCATTCACACCTACCTGTG | |
| | GCGTCGCCAGTAG | |
| 7 | ATGGAGTTTTCAAGTCCTTCCAGAGAGGAATGTCCCAAGCCTTTGA | Human |
| | GTAGGGTAAGCATCATGGCTGGCAGCCTCACAGGATTGCTTCTACT | GBA1 |
| | TCAGGCAGTGTCGTGGGCATCAGGTGCCCGCCCCTGCATCCCTAAA | codon- |
| | AGCTTCGGCTACAGCTCGGTGGTGTGTGTCTGCAATGCCACATACT | optimized |
| | GTGACTCCTTTGACCCCCCGACCTTTCCTGCCCTTGGTACCTTCAGC | nucleotide |
| | CGCTATGAGAGTACACGCAGTGGGCGACGGATGGAGCTGAGTATG | sequence |
| | GGGCCCATCCAGGCTAATCACACGGGCACAGGCCTGCTACTGACC | with |
| | CTGCAGCCAGAACAGAAGTTCCAGAAAGTGAAGGGATTTGGAGGG | reporter |
| | GCCATGACAGATGCTGCTGCTCTCAACATCCTTGCCCTGTCACCCC | sequence |
| | CTGCCCAAAATTTGCTACTTAAATCGTACTTCTCTGAAGAAGGAAT | |
| | CGGATATAACATCATCCGGGTACCCATGGCCAGCTGTGACTTCTCC | |
| | ATCCGCACCTACACCTATGCAGACACCCCTGATGATTTCCAGTTGC | |
| | ACAACTTCAGCCTCCCAGAGGAAGATACCAAGCTCAAGATACCCC | |
| | TGATTCACCGAGCCCTGCAGTTGGCCCAGCGTCCCGTTTCACTCCT | |
| | TGCCAGCCCCTGGACATCACCCACTTGGCTCAAGACCAATGGAGC | |
| | GGTGAATGGGAAGGGGTCACTCAAGGGACAGCCCGGAGACATCTA | |
| | CCACCAGACCTGGGCCAGATACTTTGTGAAGTTCCTGGATGCCTAT | |
| | GCTGAGCACAAGTTACAGTTCTGGGCAGTGACAGCTGAAAATGAG | |
| | CCTTCTGCTGGGCTGTTGAGTGGATACCCCTTCCAGTGCCTGGGCT | |
| | TCACCCCTGAACATCAGCGAGACTTCATTGCCCGTGACCTAGGTCC | |
| | TACCCTCGCCAACAGTACTCACCACAATGTCCGCCTACTCATGCTG | |
| | GATGACCAACGCTTGCTGCTGCCCCACTGGGCAAAGGTGGTACTG | |
| | ACAGACCCAGAAGCAGCTAAATATGTTCATGGCATTGCTGTACATT | |
| | GGTACCTGGACTTTCTGGCTCCAGCCAAAGCCACCCTAGGGGAGA | |
| | CACACCGCCTGTTCCCCAACACCATGCTCTTTGCCTCAGAGGCCTG | |
| | TGTGGGCTCCAAGTTCTGGGAGCAGAGTGTGCGGCTAGGCTCCTG | |
| | GGATCGAGGGATGCAGTACAGCCACAGCATCATCACGAACCTCCT | |
| | GTACCATGTGGTCGGCTGGACCGACTGGAACCTTGCCCTGAACCCC | |
| | GAAGGAGGACCCAATTGGGTGCGTAACTTTGTCGACAGTCCCATC | |
| | ATTGTAGACATCACCAAGGACACGTTTTACAAACAGCCCATGTTCT | |
| | ACCACCTTGGCCACTTCAGCAAGTTCATTCCTGAGGGCTCCCAGAG | |
| | AGTGGGGCTGGTTGCCAGTCAGAAGAACGACCTGGACGCAGTGGC | |
| | ACTGATGCATCCCGATGGCTCTGCTGTTGTGGTCGTGCTAAACCGC | |
| | TCCTCTAAGGATGTGCCTCTTACCATCAAGGATCCTGCTGTGGGCT | |
| | TCCTGGAGACAATCTCACCTGGCTACTCCATTCACACCTACCTGTG | |
| | GCGTCGCCAGGGCGGCGGCGGCAGCCACAGGTGGTGCTGCCCCGG | |
| | CTGCTGCAAGACCTTCTAG | |
| 8 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (T369M |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIIMNLLYHVV | |
| | GWTDWNLALNPEGGPNWVRNFVDSPIIVDITKDTFYKQPMFYHLGHF | |
| | SKFIPEGSQRVGLVASQKNDLDAVALMHPDGSAVVVVLNRSSKDVPL | |
| | TIKDPAVGFLETISPGYSIHTYLWRRQ | |
| 9 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (G377S |
| | LHNFSLPEEDTKLKIPLIHRALQLAQRPVSLLASPWTSPTWLKINGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVS | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 10 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (D409H |
| | LHNFSLPEEDTKLKIPLIHRALQLAQRPVSLLASPWTSPTWLKINGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKHTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 11 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIWVPMASCDFSIRTYTYADTPDDFQ | (R120W |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 12 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (V394L |
| | LHNFSLPEEDTKLKIPLIHRALQLAQRPVSLLASPWTSPTWLKINGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWLRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 13 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (R496H |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRHQ | |
| 14 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (K178T |
| | LHNFSLPEEDTKLKIPLIHRALQLAQRPVSLLASTWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 15 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (R329C |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHCL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 16 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (L444R |
| | LHNFSLPEEDTKLKIPLIHRALQLAQRPVSLLASPWTSPTWLKINGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDRDAVALMHPDGSAVVVVLNRSSKDVPL | |
| | TIKDPAVGFLETISPGYSIHTYLWRRQ | |
| 17 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (N188S |
| | LHNFSLPEEDTKLKIPLIHRALQLAQRPVSLLASPWTSPTWLKTSGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |

1. A method of increasing expression of GBA1 in a cell, the method comprising:
introducing, into a neurally differentiated cell, a recombinant adeno-associated viral (rAAV) vector comprising a promoter operably linked to a nucleic acid sequence encoding GBA1,
wherein the introducing results in increased expression of GBA1 in the cell.

2. The method of claim 1, wherein the cell comprises a variant of GBA1 associated with Parkinson's Disease.

3. A method of increasing expression of GBA1 in a cell, the method comprising:
introducing, into a neurally differentiated cell, a recombinant adeno-associated viral (rAAV) vector comprising a promoter operably linked to a nucleic acid sequence encoding GBA1,
wherein the cell comprises a variant of GBA1 associated with Parkinson's Disease, and the introducing results in increased expression of GBA1 in the cell.

4. The method of any one of claims 1-3 wherein the rAAV vector is pseudotyped with capsid proteins of an AAV serotype.

5. The method of any one of claim 1-4, wherein the capsid proteins are of the AAV2, AAV3, AAV3B, AAV3H, AAVLK-03, AAV5, AAV6, AAV7m8, AAV8, or AAV9 serotype, optionally of the AAV7m8, AAV9, or AAV-LK03 serotype.

6. The method of any one of claims 1-5, wherein the capsid proteins are of the AAV9 serotype.

7. The method of any one of claims 1-6, wherein the nucleic acid sequence encoding GBA1 is positioned between inverted terminal repeat (ITRs).

8. The method of claim 7, wherein the ITRs are of the same serotype as the capsid proteins.

9. The method of any one of claims 1-8, wherein the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter.

10. The method of any one of claims 1-9, wherein the promoter is a UBC promoter.

11. The method of any one of claims 1-10, wherein the cell exhibits decreased expression of GBA1, as compared to a reference cell, optionally as compared to a cell from a subject without Parkinson's Disease.

12. The method of any one of claims 1-11, wherein the cell exhibits reduced activity of the β-Glucocerebrosidase (GCase) enzyme encoded by GBA1 prior to being introduced with the rAAV vector, as compared to a reference cell, optionally as compared to a cell from a subject without Parkinson's Disease.

13. The method of any one of claims 1-12, wherein GBA1 is human GBA1.

14. The method of any one of claims 1-13, wherein the nucleic acid sequence comprises the sequence set forth in SEQ ID NO:6 or SEQ ID NO:7.

15. The method of any one of claims 1-14, wherein the nucleic acid sequence encodes an amino acid sequence comprising the amino acid sequence set forth in SEQ ID NO:1.

16. The method of any one of claims 2-15, wherein the variant of GBA1 comprises a single nucleotide polymorphism (SNP) that is associated with Parkinson's disease.

17. The method of claim 16, wherein the SNP is rs76763715.

18. The method of claim 17, wherein the rs76763715 is a cytosine variant.

19. The method of any one of claims 16-18, wherein the variant of GBA1 comprising a SNP encodes a serine, rather than an asparagine, at amino acid position 370 (N370S).

20. The method of claim 18 or claim 19, wherein the wild-type form of GBA1 comprises a thymine instead of the cytosine variant.

21. The method of claim 16, wherein the SNP is rs421016.

22. The method of claim 21, wherein the rs421016 is a guanine variant.

23. The method of any one of claims 16, 21, and 22, wherein the variant of GBA1 comprising the SNP encodes a proline, rather than a leucine, at amino acid position 444 (L444P).

24. The method of claim 22 or claim 23, wherein the wild-type form of GBA1 comprises an adenine instead of the guanine variant.

25. The method of claim 16, wherein the SNP is rs2230288.

26. The method of claim 25, wherein the rs2230288 is a thymine variant.

27. The method of any one of claims 16, 25, and 26, wherein the variant of GBA1 comprising the SNP encodes a lysine, rather than a glutamic acid, at position 326 (E326K).

28. The method of claim 26 or claim 27, wherein the wild-type form of GBA1 comprises a cytosine instead of the thymine variant.

29. The method of any one of claims 1-28, wherein the neurally differentiated cell is a dopaminergic neuron progenitor cell or a dopaminergic neuron.

30. The method of any one of claims 1-29, wherein the cell, optionally the dopaminergic neuron progenitor cell, is derived from a pluripotent stem cell (PSC), optionally an induced pluripotent stem cell (iPSC).

31. The method of any one of claims 1-30, wherein a plurality of the neurally differentiated cells, optionally the dopaminergic neuron progenitor cells, were differentiated from pluripotent stem cells (PSCs), optionally induced pluripotent stem cells (iPSCs), by a method comprising:
(a) performing a first incubation comprising culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

32. A method of differentiating neural cells, the method comprising:
(a) performing a first incubation comprising culturing pluripotent stem cells (PSCs) in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling;
(b) performing a second incubation comprising culturing cells of the spheroid in a substrate-coated culture vessel under conditions to neurally differentiate the cells; and
(c) introducing into the neurally differentiated cells a recombinant adeno-associated viral (rAAV) vector comprising a promoter operably linked to a nucleic acid sequence encoding GBA1, wherein the introducing results in increased expression of GBA1 in the cell.

33. The method of claim 32, wherein the cells comprise a variant of GBA1 associated with Parkinson's Disease.

34. The method of claim 32 or claim 33, wherein the cells exhibit decreased expression of GBA1, as compared to a reference cell, optionally as compared to a cell from a subject without Parkinson's Disease.

35. The method of any one of claims 32-34, wherein the cells exhibit reduced activity of the β-Glucocerebrosidase (GCase) enzyme encoded by GBA1 prior to being introduced with the rAAV vector, as compared to a reference cell, optionally as compared to a cell from a subject without Parkinson's Disease.

36. The method of any one of claims 31-35, wherein the cells are exposed to the inhibitor of TGF-β/activin-Nodal signaling up to a day at or before day 7.

37. The method of any one of claims 31-36, wherein the cells are exposed to the inhibitor of TGF-β/activin-Nodal beginning at day 0 and through day 6, inclusive of each day.

38. The method of any one of claims 31-37, wherein the cells are exposed to the at least one activator of SHH signaling up to a day at or before day 7.

39. The method of any one of claims 31-38, wherein the cells are exposed to the at least one activator of SHH signaling beginning at day 0 and through day 6, inclusive of each day.

40. The method of any one of claims 31-39, wherein the cells are exposed to the inhibitor of BMP signaling up to a day at or before day 11.

41. The method of any one of claims 31-40, wherein the cells are exposed to the inhibitor of BMP signaling beginning at day 0 and through day 10, inclusive of each day.

42. The method of any one of claims 31-41, wherein the cells are exposed to the inhibitor of GSK3β signaling up to a day at or before day 13.

43. The method of any one of claims 31-42, wherein the cells are exposed to the inhibitor of GSK3β signaling beginning at day 0 and through day 12, inclusive of each day.

44. The method of any one of claims 31-43, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

45. The method of claim 44, wherein the cells are exposed to BAGCT and the inhibitor of Notch signaling beginning on day 11.

46. The method of claim 44 or claim 45, wherein the cells are exposed to BAGCT and the inhibitor of Notch signaling beginning at day 11 and until harvest of the neurally differentiated cells, optionally until day 18, optionally until day 20, or optionally until day 25.

47. The method of any one of claims 31-46, wherein the inhibitor of TGF-β/activin-Nodal signaling is SB431542.

48. The method of any one of claims 31-47, wherein the at least one activator of SHH signaling is SHH or purmorphamine.

49. The method of any one of claims 31-48, wherein the inhibitor of BMP signaling is LDN193189.

50. The method of any one of claims 31-49, wherein the inhibitor of GSK3β signaling is CHIR99021.

51. The method of any one of claims 31-50, wherein the cells are introduced with the rAAV vector between about day 14 and about day 20, optionally on day 16 or day 18.

52. The method of any one of claims 31-51, wherein the cells are introduced with the rAAV vector on about day 16.

53. The method of any one of claims 31-52, wherein the cells are harvested between about day 18 and about day 25, optionally at day 18 or day 20.

54. The method of any one of claims 31-53, wherein the cells are harvested on about day 20.

55. The method of any one of claims 31-40, wherein the neurally differentiated cell was cryopreserved and subsequently thawed prior to the introducing.

56. The method of any one of claims 31-55, wherein the neurally differentiated cell was thawed about 1 day prior to the introducing.

57. The method of any one of claims 31-56, further comprising cryopreserving the neurally differentiated cell prior to the introducing.

58. The method of claim 57, wherein the cryopreserving comprises formulating the neurally differentiated cell with a cryoprotectant.

59. The method of any one of claims 45-58, wherein the PSC is an induced pluripotent stem cell (iPSC) derived from a non-pluripotent cell from a subject.

60. The method of claim 59, wherein the non-pluripotent cell is a fibroblast.

61. The method of claim 59 or claim 60, wherein the subject has Parkinson's disease or Gaucher's disease.

62. The method of any of claims 59-61, wherein the subject has Parkinson's disease.

63. A cell produced by the method of any one of claims 1-62.

64. A modified neurally differentiated cell comprising an exogenous deoxyribonucleic acid (DNA) sequence encoding GBA1, wherein the DNA sequence is episomal in the cell.

65. The neurally differentiated cell of claim 64, wherein the DNA is encoded by an episomal vector.

66. The neurally differentiated cell of claim 64 or claim 65, wherein the episomal vector is an AAV vector.

67. The neurally differentiated cell of any one of claims 64-66, wherein the cell comprises a variant of GBA1 associated with Parkinson's Disease.

68. The cell of any one of claims 63-67, wherein the cell expresses EN1 and CORIN.

69. The cell of any one of claims 63-68, wherein the cell is a committed dopaminergic precursor cell.

70. The cell of any one of claims 63-69, wherein the cell is formulated with a cryoprotectant.

71. A therapeutic composition comprising the cell of any of claims 63-70.

72. The therapeutic composition of claim 71, wherein cells of the composition express EN1 and CORIN and less than 10% of the total cells in the composition express TH.

73. The therapeutic composition of claim 71 or claim 72, wherein less than 5% of the total cells in the composition express TH.

74. The therapeutic composition of any one of claims 71-73, further comprising a cryoprotectant.

75. A method of treatment, comprising administering to a subject the therapeutic composition of any one of claims 71-74.

76. The method of claim 75, wherein the cells of the therapeutic composition are autologous to the subject.

77. The method of claim 75 or claim 76, wherein the subject has a disease or disorder associated with reduced GCase activity.

78. The method of any one of claims 75-77, wherein the subject has Gaucher's disease.

79. The method of any one of claims 75-77, wherein the subject has a Lewy body disease (LBD).

80. The method of claim 79, wherein the LBD is Parkinson's disease, Parkinson's disease dementia, or dementia with Lewy bodies (DLB).

81. The method of any one of claims 75-77, 79, and 80, wherein the subject has Parkinson's disease.

82. The method of any one of claims 75-81, wherein the administering comprises delivering the cells of the therapeutic composition by stereotactic injection.

83. The method of any one of claims 75-82, wherein the administering comprises delivering the cells of the therapeutic composition through a catheter.

84. The method of claim 82 or claim 83, wherein the cells of the therapeutic composition are delivered to the striatum of the subject.

85. Use of the therapeutic composition of any one of claims 71-74, for the treatment of a disease or disorder associated with reduced GCase activity.

86. Use of the therapeutic composition of any one of claims 71-74, for the treatment of Gaucher's disease.

87. Use of the therapeutic composition of any one of claims 71-74, for the treatment of a Lewy body disease (LBD).

88. The use of claim 87, wherein the LBD is Parkinson's disease, Parkinson's disease dementia, or dementia with Lewy bodies (DLB).

89. Use of the therapeutic composition of any one of claims 71-74, for the treatment of Parkinson's Disease.

90. A recombinant adeno-associated viral (rAAV) nucleic acid vector for increasing expression of GBA1 in a cell, the vector comprising a promoter operably linked to a nucleic acid sequence encoding GBA1, wherein the cell exhibits (i) reduced activity of the β-Glucocerebrosidase (GCase) enzyme encoded by GBA1 and/or (ii) reduced expression of GBA1 prior to being introduced with the rAAV vector, optionally as compared to a reference cell from a subject without Parkinson's Disease.

91. The rAAV vector of claim 90, wherein the cell comprises a variant of GBA1 associated with Parkinson's Disease.

92. The rAAV vector of claim 91, wherein the variant of GBA1 comprises a single nucleotide polymorphism (SNP) that is associated with Parkinson's disease.

93. The rAAV vector of claim 92, wherein the SNP is rs76763715.

94. The rAAV vector of claim 93, wherein the rs76763715 is a cytosine variant.

95. The rAAV vector of any one of claims 90-94, wherein the variant of GBA1 comprising a SNP encodes a serine, rather than an asparagine, at amino acid position 370 (N370S).

96. The rAAV vector of claim 94 or claim 95, wherein the wild-type form of GBA1 comprises a thymine instead of the cytosine variant.

97. The rAAV vector of claim 92, wherein the SNP is rs421016.

98. The rAAV vector of claim 97, wherein the rs421016 is a guanine variant.

99. The rAAV vector of any one of claims 92, 97, and 98, wherein the variant of GBA1 comprising the SNP encodes a proline, rather than a leucine, at amino acid position 444 (L444P).

100. The rAAV vector of claim 98 or claim 99, wherein the wild-type form of GBA1 comprises an adenine instead of the guanine variant.

101. The rAAV vector of claim 92, wherein the SNP is rs2230288.

102. The rAAV vector of claim 101, wherein the rs2230288 is a thymine variant.

103. The rAAV vector of any one of claims 92, 101, and 102, wherein the variant of GBA1 comprising the SNP encodes a lysine, rather than a glutamic acid, at position 326 (E326K).

104. The rAAV vector of claim 102 or claim 103, wherein the wild-type form of GBA1 comprises a cytosine instead of the thymine variant.

105. The rAAV vector of any one of claims 90-104, wherein the cell is a dopaminergic neuron progenitor cell or a dopaminergic neuron.

106. The rAAV vector of any one of claims 90-105, wherein the cell, optionally the dopaminergic neuron progenitor cell, is derived from a pluripotent stem cell (PSC), optionally an induced pluripotent stem cell (iPSC).

107. The rAAV vector of any one of claims 90-106, wherein a plurality of the cell, optionally the dopaminergic neuron progenitor cell, were differentiated from pluripotent stem cells (PSCs), optionally induced pluripotent stem cells (iPSCs), by a method comprising:
(a) performing a first incubation comprising culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

108. The rAAV vector of claim 107, wherein the cells are exposed to the inhibitor of TGF-β/activin-Nodal signaling and the at least one activator of SHH signaling up to a day at or before day 7.

109. The rAAV vector of claim 107 or claim 108, wherein the cells are exposed to the inhibitor of BMP signaling up to a day at or before day 11.

110. The rAAV vector of any one of claims 107-109, wherein the cells are exposed to the inhibitor of GSK3β signaling up to a day at or before day 13.

111. The rAAV vector of any one of claims 107-110, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.
